(19) United States
(12) Patent Application Publication
(10) Pub. No.: US 2006/0039866 A1
(43) Pub. Date: Feb. 23, 2006

(54) Method for treating sleep-related breathing disorders

(75) Inventors: Srinivas G. Rao, Encinitas, CA (US); Jay D. Kranzler, La Jolla, CA (US); Jeffery J. Anderson, San Diego, CA (US)

Correspondence Address: WILSON SONSINI GOODRICH & ROSATI, 650 PAGE MILL ROAD, PALO ALTO, CA, 94304-1050, US

(73) Assignee: Cypress Bioscience, Inc.

(21) Appl. No.: 11/208,205

(22) Filed: Aug. 19, 2005

Related U.S. Application Data
(60) Provisional application No. 60/603,367, filed on Aug. 20, 2004.
(60) Provisional application No. 60/607,160, filed on Sep. 3, 2004.
(60) Provisional application No. 60/609,618, filed on Sep. 14, 2004.
(60) Provisional application No. 60/612,954, filed on Sep. 24, 2004.
(60) Provisional application No. 60/616,661, filed on Oct. 7, 2004.
(60) Provisional application No. 60/619,571, filed on Oct. 15, 2004.
(60) Provisional application No. 60/621,145, filed on Oct. 22, 2004.
(60) Provisional application No. 60/659,708, filed on Mar. 8, 2005.

(51) Int. Cl. A61K 31/7008; A61K 31/551; A61K 31/506; A61L 9/04; A61K 31/42

(52) U.S. Cl. 424/45; 514/23; 514/220; 514/252.15; 514/379

(57) ABSTRACT

Compositions and methods for the treatment of sleep related breathing disorders are provided. Compositions include mirtazapine in combination with other active pharmaceutical ingredients, such as zonisamide, topiramate or modafinil. The treated sleep related breathing disorders include sleep apnea and sleep hypopnea. In some embodiments, the pharmaceutical compounds are used as adjuvant therapy with positive airway pressure (PAP) therapy, thereby lowering the pressure required to maintain airway patency during PAP therapy.

### CROSS REFERENCE TO RELATED APPLICATIONS

This application claims priority under 35 U.S.C. § 119(e) from the following U.S. provisional patent applications: 60/603,367, filed on Aug. 20, 2004; 60/607,160, filed on Sep. 3, 2004; 60/609,618, filed on Sep. 14, 2004; 60/612,954, filed on Oct. 13, 2004; 60/616,661, filed on Oct. 7, 2004; 60/619,571, filed on Oct. 15, 2004; 60/621,145, filed on Oct. 22, 2004; and 60/659,708, filed on Jan. 19, 2005. Each of these provisional applications is expressly incorporated herein in its entirety.

### FIELD OF THE INVENTION

This invention generally relates to compositions and methods for the pharmacological treatment of sleep related breathing disorders and for the pharmacological improvement of mechanical methods of treating sleep disorders such as sleep apnea.

### BACKGROUND OF THE INVENTION

Over the past several years much effort has been devoted to the study of a discrete group of breathing disorders that occur primarily during sleep. The consequences of these breathing disorders tend to persist throughout the waking hours. For example, those who suffer from such breathing disorders tend to experience substantial daytime sleepiness, which can cause substantial economic losses, not to mention pervasive safety issues, as sufferers are likely to be less attentive to work and more prone to accidents. Sleep related breathing disorders are characterized by repetitive reduction in breathing (hypopnea), periodic cessation of breathing (apnea), or a continuous or sustained reduction in ventilation during sleep. Sleep related breathing disorders are thus distinguished from other breathing disorders, such as reactive airway disease and asthma, which may occur during sleep, but are not associated with sleep in that they may arise whether the patient is awake or asleep. Sleep related breathing disorders also do not include seizure-induced apnea, which does not primarily affect sleeping individuals.

Sleep apnea is defined as an intermittent cessation of airflow at the nose and mouth during sleep. By convention, apneas of at least 10 seconds in duration have been considered important; but in most sufferers the apneas are 20-30 seconds in duration and may be as long as 2-3 minutes. While there is some uncertainty as to the minimum number of apneas that should be considered clinically important, by the time most sufferers come to attention of the medical community they have at least 10 to 15 events per hour of sleep.

Sleep apneas have been classified into three types: central, obstructive, and mixed. In central sleep apnea, the neural drive to all respiratory muscles is transiently abolished. In obstructive sleep apneas (OSAs), airflow ceases despite continuing respiratory drive because of occlusion of the oropharyngeal airway. Mixed apneas, which consist of a central apnea followed by an obstructive component, are a variant of obstructive sleep apnea. The most common type of apnea is obstructive sleep apnea.

Hypopneas are episodes of shallow breathing during which airflow is decreased by at least 50%. Like apnea, hypopnea is subdivided as being obstructive, central, or mixed. Obstructive hypopneas are episodes of partial upper airway obstruction. In central hypopnea, breathing effort and airflow are both decreased. Mixed hypopneas have both central and obstructive components. Individuals with obstructive sleep apnea syndrome (OSAS) have pathologic degrees of obstructive apnea, obstructive hypopnea, or both.

Currently, the most common and most effective treatments for adults with sleep apnea and other sleep related breathing disorders are mechanical forms of therapy that deliver positive airway pressure (PAP). Under PAP treatment, an individual wears a tight-fitting plastic mask over the nose when sleeping. The mask is attached to a compressor, which forces air into the nose creating a positive pressure within the patient's airways. The principle of the method is that pressurizing the airways provides a mechanical “splinting” action, which prevents airway collapse, and therefore, obstructive sleep apnea. Although an effective therapeutic response is observed in most patients who undergo PAP treatment, many patients cannot tolerate the apparatus or pressure and refuse treatment. Moreover, recent covert monitoring studies clearly demonstrate that long-term compliance with PAP treatment is very poor.

A variety of upper airway and craniofacial surgical procedures have been attempted for treatment of OSAS. Adenotonsillectomy appears to be an effective cure for OSAS in many children, but upper airway surgery is rarely curative in adult patients with OSAS. Surgical “success” is generally taken to be a 50% reduction in apnea incidence and there are no useful screening methods to identify the individuals that would benefit from the surgery versus those who would not derive a benefit.

Pharmacological treatments of several types have been attempted in patients with sleep apnea but, thus far, none have proven to be generally useful. A recent systematic review of these attempts is provided by Hudgel [J. Lab. Clin. Med., 126:13-18 (1995)]. A number of compounds have been tested because of their expected respiratory stimulant properties. These include (1) acetazolamide, a carbonic anhydrase inhibitor that produced improvement in apnea/hypopnea frequency and decreased the frequency of 4% desaturation; Whyte et al., Role of Protriptyline and Acetazolamide in the Sleep Apnea/Hypopnea Syndrome, Sleep, 1998, 11(5), 463-472; (2) medroxyprogesterone, a progestin that has demonstrated no consistent benefit in OSAS; and (3) theophylline, a compound usually used for the treatment of asthma, which may benefit patients with central apnea but appears to be of no use in adult patients with obstructive apnea.

Other attempted pharmacological treatment includes the administration of adenosine, adenosine analogs and adenosine reuptake inhibitors (U.S. Pat. No. 5,075,290). Specifically, adenosine, which is a ubiquitous compound within the body and which levels are elevated in individuals with OSAS, has been shown to stimulate respiration and is somewhat effective in reducing apnea in an animal model of sleep apnea.

Other possible pharmacological treatment options for OSAS include agents that stimulate the brain activity or are opioid antagonists. Specifically, since increased cerebral spinal fluid opioid activity has been identified in OSAS, it is a logical conclusion that central stimulants or opioid antagonists would be a helpful treatment of OSAS. In reality, doxapram, which stimulates the central nervous system and carotid body chemoreceptors, was found to decrease the length of apneas but did not alter the average arterial oxygen saturation in individuals with obstructive sleep apnea. The opioid antagonist naloxone, which is known to stimulate ventilation was only slightly helpful in individuals with obstructive sleep apnea.

Because OSAS is strongly correlated with the occurrence of hypertension, agents such as angiotensin-converting enzyme (ACE) inhibitors may be of benefit in treating OSAS sufferers with hypertension but this does not appear to be a viable treatment for OSAS itself.

Mirtazapine has been taught as a monotherapy or in combination with a selective serotonin reuptake inhibitor in a rat model. Andrews, U.S. Pat. No. 6,303,595. However, while mirtazapine, alone or in combination with an SSRI, appeared effective the rat model, the efficacy of mirtazapine monotherapy and mirtazapine plus SSRI combination therapy have not been demonstrated in humans.

Finally, several agents that act on neurotransmitters and neurotransmitter systems involved in respiration have been tested in individuals with OSAS. Most of these compounds have been developed as anti-depressant medications that work by increasing the activity of monoamine neurotransmitters including norepinephrine, dopamine, and serotonin. Protriptyline, a tricyclic anti-depressant, has been tested in several small trials with variable results and frequent and significant side effects. As serotonin may promote sleep and stimulate respiration, tryptophan, a serotonin precursor and selective serotonin reuptake inhibitors have been tested in individuals with OSAS. While a patent has been issued for the use of the serotonin reuptake inhibitor, fluoxetine (U.S. Pat. No. 5,356,934), initial evidence suggests that these compounds may yield measurable benefits in only a small subset of individuals with OSAS. Therefore in view of the fact that the only viable treatment for individuals suffering from sleep related breathing disorders is a mechanical form of therapy (PAP) for which patient compliance is low, and that hopes for pharmacological treatments have yet to come to fruition, there remains a need for simple pharmacologically-based treatments that would offer benefits to a broad base of individuals suffering from a range of sleep related breathing disorders. There also remains a need for a viable treatment of sleep related breathing disorders that would lend itself to a higher rate of patient compliance.

It is therefore an object of the present invention to provide an effective pharmaceutical therapy for sleep related breathing disorders and the sequelae of sleep apnea.

It is also an object of the present invention to provide pharmaceutical compositions and methods of using such compositions to improve patient tolerance of positive airway pressure therapy. Such improved response includes improving patient compliance, improving patient comfort, increasing efficacy of the therapy and decreasing the applied pressure needed to maintain airway patency.

The present invention meets the foregoing needs and objectives, and provides related advantages as well.

### BRIEF SUMMARY OF THE INVENTION

The foregoing needs and objectives are satisfied by embodiments of the present invention, which provide a method of treating a sleep related breathing disorder with a combination of mirtazapine and a second pharmaceutically active ingredient, which provides a combination of beneficial effects to the patient. In particular, the second pharmaceutically active ingredient improves upper airway muscle tone during sleep and stabilizes respiratory drive. In particular embodiments, the second pharmaceutically active ingredient is zonisamide.

The needs and objectives outlined above are further met by embodiments of the invention, which provide a composition comprising mirtazapine and a second pharmaceutically active ingredient, which provides a combination of beneficial effects to the patient. In particular, the second pharmaceutically active ingredient improves upper airway muscle tone during sleep and stabilizes respiratory drive. In particular embodiments, the second pharmaceutically active ingredient is zonisamide.

The foregoing needs and objectives are further addressed by embodiments of the invention, which provide a kit comprising a first dosage form comprising mirtazapine and a second dosage form comprising a second pharmaceutically active ingredient, which provides a combination of beneficial effects to the patient. In particular, the second pharmaceutically active ingredient improves upper airway muscle tone during sleep and stabilizes respiratory drive. In particular embodiments, the second pharmaceutically active ingredient is zonisamide.

Other needs and objectives are satisfied by embodiments of the present invention, which provide a method of treating a sleep related breathing disorder with a combination of mirtazapine and a second pharmaceutically active ingredient, which stabilizes respiratory drive. In particular embodiments, the second pharmaceutically active ingredient is selected from the group consisting of topiramate, zonisamide and a 5HT1A.

In some embodiments, the daily dose of mirtazapine for the treatment of sleep related breathing disorders is in the range of about 5 to about 50 mg per day, while the daily dose of zonisamide is in the range of about 25 to about 200 mg per day.

The needs and objectives outlined above are further met by embodiments of the invention, which provide a composition comprising mirtazapine and a second pharmaceutically active ingredient, which stabilizes respiratory drive. In particular embodiments, the second pharmaceutically active ingredient is selected from the group consisting of consisting of topiramate, zonisamide and a 5HT1A.

The foregoing needs and objectives are further addressed by embodiments of the invention, which provide a kit comprising a first dosage form comprising mirtazapine and a second dosage form comprising a second pharmaceutically active ingredient, which stabilizes respiratory drive. In particular embodiments, the second pharmaceutically active ingredient is selected from the group consisting of consisting of topiramate, zonisamide and a 5HT1A.

The foregoing needs and objectives are satisfied by embodiments of the present invention, which provide a method of treating a sleep related breathing disorder with a combination of mirtazapine and a second pharmaceutically active ingredient, which treats one of the sequelae of sleep apnea, such as excessive daytime drowsiness. In particular embodiments, the second pharmaceutically active ingredient is selected from the group consisting of amantadine, bupropion, modafinil, r-modafinil, SDZ-NVI-085 and amphetamine.

The needs and objectives outlined above are further met by embodiments of the invention, which provide a composition comprising mirtazapine and a second pharmaceutically active ingredient, which treats one of the sequelae of sleep apnea, such as excessive daytime drowsiness. In particular embodiments, the second pharmaceutically active ingredient is selected from the group consisting of amantadine, bupropion, modafinil, r-modafinil, SDZ-NVI-085 and amphetamine.

The foregoing needs and objectives are further addressed by embodiments of the invention, which provide a kit comprising a first dosage form comprising mirtazapine and a second dosage form comprising a second pharmaceutically active ingredient, which treats one of the sequelae of sleep apnea, such as excessive daytime drowsiness. In particular embodiments, the second pharmaceutically active ingredient is selected from the group consisting of amantadine, bupropion, modafinil, r-modafinil, SDZ-NVI-085 and amphetamine.

The present invention further meets the foregoing needs and objectives by providing a method of using a composition comprising mirtazapine to improve patient tolerance of positive airway pressure (PAP) therapy. The method includes administering to a patient undergoing PAP therapy an amount of a composition comprising mirtazapine, which is sufficient to improve patient tolerance of PAP therapy. In some embodiments, mirtazapine is provided in a daily dose of about 5 to about 50 mg per day. In particular embodiments, the amount of mirtazapine given is sufficient to permit use of a lower PAP pressure than would be required without mirtazapine therapy.

The invention further provides compositions, and methods of use thereof, for the prevention or treatment of sleep related breathing disorders such as sleep apnea, upper airway resistance syndrome, and conditions associated with sleep related breathing disorders, such as excessive daytime sleepiness and weight gain. The method of treating sleep apnea, upper airway resistance syndrome, etc. involves a combination of: (a) increasing the tone of upper airway muscles during sleep, (b) stabilizing respiratory drive, (c) increasing deep (Slow Wave) sleep, and (d) suppressing REM sleep. In particular, the invention provides a method of (a) increasing the tone of upper airway muscles during sleep, (b) stabilizing respiratory drive, (c) increasing deep (Slow Wave) sleep, and (d) suppressing REM sleep, comprising administering to a patient a therapeutically effective amount of a combination of mirtazapine and a second pharmaceutically active ingredient, which, in combination with mirtazapine, is effective to increase the tone of upper airway muscles during sleep, stabilize respiratory drive, increase deep sleep and suppress REM sleep.

### DETAILED DESCRIPTION OF THE INVENTION

The invention provides therapeutic compositions and methods for the treatment of sleep related breathing disorders. Sleep related breathing disorders are those breathing disorders that are predominantly experienced during sleep, such as apnea, snoring and hypopnea. As discussed above, apnea, snoring and hypopnea can be serious conditions, giving rise to several symptoms that range in severity from inconvenient to life threatening.

In the context of the present invention, “treating a sleep related breathing disorder” (including its grammatical variants), unless otherwise modified, means preventing or ameliorating one or more symptoms or sequelae of a sleep related breathing disorder. The symptoms of a sleep related breathing disorder include snoring, hypoxia, interrupted sleep, intermittent cessation of breathing during sleep (apnea) and decreased ventilation during sleep (hypopnea). Sequelae of apnea include excessive daytime drowsiness due to intermittent interruptions in sleep, reduced psychomotor functioning due to hypoxia, hypertension, cardiopulmonary disease and other disorders and conditions arising out of long-term hypoxia and concomitant ischemia.

As identified by the present inventors, in order to treat sleep related breathing disorders, a therapeutic agent must: (a) increase the tone of upper airway muscles in the patient during sleep, (b) stabilize the patient's respiratory drive, (c) increase deep (Slow Wave) sleep, and (d) suppress REM sleep. These effects are listed in order of importance, the first two being of greater relative importance than the latter two. The present invention thus provides an effective treatment for sleep related breathing disorders by providing a combination of pharmaceutically active ingredients, which, in combination, provide all of these effects. As it has been found that 5HT3 antagonists, such as mirtazapine, failed to provide one or more of these effects in humans, the present invention provides a therapeutic approach combining mirtazapine and at least one other pharmaceutically active ingredient. In particular, 5HT3 antagonists have proven to be lacking in beneficial effect on stabilization of respiratory drive in humans. Thus, one combination approach of the present invention is focused on providing a combination therapy, wherein the second pharmaceutically active ingredient (or combination of ingredients) provide at least one of the pharmaceutical activities (a)-(d) that mirtazapine lacks in humans. In particular, a combination approach of the present invention provides mirtazapine and a second pharmaceutically active ingredient that provides respiratory drive stabilization in humans.

In some embodiments, the second pharmaceutically active ingredient provides a combination of activities, including increasing the tone of upper airway muscles during sleep and stabilizing respiratory drive. Zonisamide, a carbonic anhydrase inhibitor, has been identified as a compound that provides this effect. Thus, the invention provides a combination therapy comprising a therapeutically effective amount of a combination of mirtazapine and zonisamide. In some embodiments, mirtazapine and zonisamide can be combined in the same dosage form. In other embodiments, mirtazapine and zonisamide may be administered as separate dosage forms. In particular embodiments, mirtazapine and zonisamide are present in separate dosage forms that are combined in a kit. Thus, mirtazapine and zonisamide may be co-administered in at the same time or at different times during the course of treatment.

In some embodiments, the second pharmaceutically active ingredient provides primarily respiratory drive stabilization. Exemplary compounds that stabilize respiratory drive include: topiramate, amantadine, bupropion, modafinil, r-modafinil, a 5HT1A agonist and SDZ-NVI-085. In some embodiments, mirtazapine and the compound that stabilizes respiratory drive in humans can be combined in the same dosage form. In other embodiments, mirtazapine and the compound that stabilizes respiratory drive in humans may be administered as separate dosage forms. In particular embodiments, mirtazapine and the compound that stabilizes respiratory drive in humans are present in separate dosage forms that are combined in a kit. Thus, mirtazapine and the compound that stabilizes respiratory drive in humans may be co-administered in at the same time or at different times during the course of treatment. Thus, mirtazapine may be co-administered with topiramate, a 5HT1A agonist such as buspirone, or zonisamide.

A number of sequelae of sleep related breathing disorders have been identified. In some embodiments, a suitable treatment for sleep related breathing disorders would alleviate one or more of these sequelae. Thus, in some embodiments, the present invention provides combination therapy of mirtazapine with a compound that provides a beneficial effect in treating one or more sequelae of sleep related breathing disorders, such as sleep apnea. In particular embodiments, the compound that treats one or more sequelae of a sleep related breathing disorder are selected from the group consisting of amantadine, bupropion, modafinil, r-modafinil, SDZ-NVI-085 and amphetamine. In some embodiments, mirtazapine and the compound that treats one or more sequelae of a sleep related breathing disorder can be combined in the same dosage form. In other embodiments, mirtazapine and the compound that treats one or more sequelae of a sleep related breathing disorder may be administered as separate dosage forms. In particular embodiments, mirtazapine and the compound that treats one or more sequelae of a sleep related breathing disorder are present in separate dosage forms that are combined in a kit. Thus, mirtazapine and the compound that treats one or more sequelae of a sleep related breathing disorder may be co-administered in at the same time or at different times during the course of treatment.

A “therapeutic effect” (including its grammatical variants), unless otherwise modified, means amelioration or prevention of a sleep related breathing disorder or one or more sequelae of a sleep related breathing disorder.

As used herein, the conjunction “or,” unless otherwise modified, is intended in its inclusive sense, such that “or” means “and/or”. Thus a list of “A or B” means, “A, B or A plus B.”

As used herein, the article “a”, unless otherwise modified, is intended to include more than one. Thus, “a compound,” unless otherwise modified, means “at least one compound.”

A patient, as used herein, is a human subject to whom a composition or dosage form of the invention is administered. Such subjects include those suffering from one or more sleep related breathing disorders, as defined above. Such subjects also include those who are judged by a physician to be at risk for one or more sleep related breathing disorders, owing to advanced age, obesity or medication.

I. Compositions: The present invention provides pharmaceutical compositions for the treatment of sleep related breathing disorders. In some embodiments, a therapeutically effective amount of a composition is an amount effective to (a) increase the tone of upper airway muscles in a patient during sleep, (b) stabilize respiratory drive in a patient, (c) increase deep (Slow Wave) sleep, and (d) suppress REM sleep. In some embodiments, a therapeutically effective amount of a composition is an amount effective to reduce snoring, reduce the incidence of apnea or hypopnea, improve blood oxygenation of the patient during sleep, decrease the incidence of a patient's intermittent waking due to apnea or hypopnea, or improve a patient's alertness during the day.

In particular embodiments, the invention provides a unit dosage form of mirtazapine in combination with one or more other active pharmaceutical ingredients described herein. The composition may also comprise one or more inactive ingredients, which include pharmaceutically acceptable carriers, excipients or diluents, as described in more detail below. Mirtazapine may also be combined with a pharmaceutically acceptable acid or base to form a pharmaceutically acceptable salt. In some embodiments, the daily dose of mirtazapine for the treatment of sleep related breathing disorders is in the range of about 5 to about 50 mg per day.

Exemplary active ingredients that may be used along with mirtazapine include zonisamide. In some embodiments, zonisamide provides additive or complementary effects in combination with mirtazapine. Thus, some unit dosage forms according to the invention comprise a therapeutically effective amount of a combination of mirtazapine and zonisamide. In some embodiments, the daily dose of mirtazapine for the treatment of sleep related breathing disorders is in the range of about 5 to about 50 mg per day, while the daily dose of zonisamide is in the range of about 25 to about 200 mg per day.

A. Compounds Used to Treat Sleep related Breathing Disorders. Below are set forth a number of compounds that can be used for the treatment of one or more sleep related breathing disorders.

The compounds set forth below can be used alone or in combination with other compounds to produce additive, complementary or synergistic effects in the treatment of sleep related breathing disorders. In some embodiments, combinations of compounds can be prepared as mixtures. In other embodiments, the combinations of compounds can be prepared as unit dosage forms, wherein at least two pharmaceutically active compounds are isolated in separate phases. In still further embodiments, a combination of compounds can be prepared as a kit, in which a first compound is prepared in first dosage form and a second compound is prepared in a dosage form that is separate from the first dosage form. In some embodiments, such kits include packaging, such as blister packs, that divide the pharmaceutically active ingredients into convenient doses.

1. Drugs that Act as 5HT1A Serotonin Receptor Agonists, Serotonin Reuptake Inhibitors, and 5HT2A/2C Serotonin Receptor Antagonists. In one embodiment, the sleep related breathing disorders are treated with a drug that acts as a 5HT1A serotonin receptor agonist, serotonin reuptake inhibitor, and 5HT2A/2C serotonin receptor antagonist (5HT1A sertotonin receptor agonist/serotonin reuptake inhibitor/5HT2A/2C serotonin receptor antagonist or 5HT1A agonist/SRI/5HT2A/2C antagonist). Useful compounds include trazadone or alkyl derivatives thereof, such as MER 810. In a preferred embodiment, the disorders are treated with trazadone or an alkyl derivative thereof (i.e. MER 810) in combination with a norepinephrine reuptake inhibitor, norepinephrine/serotonin reuptake inhibitor, alpha-1 agonist, alpha-2 antagonist, quinacrine, a compound that acts as a NERI and a 5HT3 serotonin receptor antagonist, such as MC1225 (4-(2-Fluorophenyl)-6-methyl-2-(1-piperazinyl)thieno[2,3-D]pyrimidine monohydrate hydrochloride), an SSRI/5HT1A partial agonist, an SRI/5HT3 antagonist, or an SSRI.

MER 810 is quite effective in treating OSA, an unusual result in light of this agent's prominent 5-HT 2A/2C antagonist activity. The latter has been associated with worsening OSA symptoms due to reduction in upper airway tone in animal models. However, this pharmacology may also increase SWS and increase respiratory drive via peripheral effects, while its serotonin (5-HT) reuptake inhibition and 5-HT1A agonist activities decrease REM sleep. The 5-HT1A agonist activity also increases respiratory drive and may increase upper airway muscle tone (Homer). Adding a noradrenergic alpha1 agonist, alpha2 antagonist or reuptake inhibitor bolsters the latter effect.

Trazadone and derivatives or analogs thereof, having the following general formula, are useful in the treatment of sleep related breathing disorders.

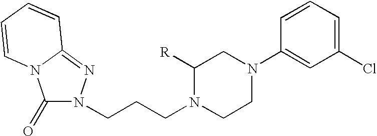

wherein R is hydrogen or an alkyl group having from 1 to 3 carbons. A preferred embodiment is the following compound wherein R is methyl (MER 810).

These compounds work by binding to the serotonin receptor as well as inhibiting serotonin reuptake. The affinity of MER 810 for the 5HT1A serotonin receptor has been demonstrated to be 7.07(pKi).

2. Norepinephrine Reuptake Inhibitors (NERI) and Norepinephrine/Serotonin Reuptake Inhibitors (NSRI). Useful compounds include any drug that is selective for inhibiting norepinephrine reuptake (NERI) and any compound that inhibits the reuptake of both norepinephrine and serotonin (NSRI). Preferred compounds in the former category include atomoxetine, reboxetine, tomoxetine, and bicifadine. Preferred compounds in the latter category include milnacipran, venlafaxine, desipramine, duloxetine.

In one embodiment, NERIs, especially brain selective compounds such as atomoxetine and reboxetine, can be used in combination with a 5HT2/5HT3 antagonist/alpha-2 antagonist to reduce the excessive daytime drowsiness associated with 5HT2/5HT3 antagonist/alpha-2 antagonist use for the treatment of sleep related breathing disorders. NERIs can often produce cardiovascular side effects, such as increases in blood pressure and heart rate. Those compounds that concentrate in the brain, such as atomoxetine and reboxetine, have fewer cardiovascular effects, which can be important for the treatment of patients with sleep related breathing disorders.

3. Selective Serotonin Reuptake Inhibitors (SSRI). Useful SSRI's include any compound that is selective for inhibiting serotonin (5HT) reuptake. Preferred SSRIs include sertraline and citalopram, due to their benign effects upon sleep architecture.

4. Alpha-2 Adrenergic Receptor Antagonists. In another embodiment, the sleep related breathing disorders are treated with alpha-2 adrenergic receptor antagonists (alpha-2 antagonists). In a preferred embodiment, the disorders are treated with an alpha 2 antagonist in combination with a SSRI and/or 5-HT1A agonist.

Useful alpha-2 adrenergic receptor antagonists include any drug that binds to but does not activate alpha-adrenergic receptors, thereby blocking the actions of endogenous or exogenous adrenergic agonists. Preferred alpha-2 antagonists include fipamezole and dexeferoxan.

Alpha-2 antagonists have the ability to bolster upper airway tone, an effect augmented by the addition of an SSRI drug. Furthermore, the latter also suppresses REM sleep, further improving the therapeutic profile of alpha 2 antagonists. Preferred SSRIs include sertraline and citalopram, due to their benign effects upon sleep architecture). An alternative approach is to combined fipamezole with a 5-HT1a agonist.

5. Alpha-1 Adrenergic Receptor Agonists. Useful alpha-1 adrenergic receptor agonists (alpha-1 agonists) include any drug that binds to and activates the alpha-1 adrenergic receptor. A preferred compound is SDZ-NVI-085, [( )-(4aR, 10aR)-3,4,4a,5,10,10a-hexahydro-6-methoxy-4-methyl-9-methylthio-2H-naphth 2,3,b-1,4-oxazine HCl] which is a centrally active alpha-1 agonist that can improve OSA without undue cardiovascular side effects. SDZ-NVI-085 also acts as a 5HT2A serotonin receptor antagonist, which aids in its efficacy in the treatment of sleep related breathing disorders. Other alpha-1 agonists include modafinil (benzhydrylsulphinylacetamide) and adrafinil (benzhydrylsulphinyl-acetohydroxamic acid).

In one embodiment, alpha-1 agonists are used to treat excessive daytime sleepiness associated with sleep related breathing disorders. In another embodiment, alpha-1 agonists are used in combination with drugs that improve the hypoxic index such as 5HT1A agonist/SRI/5HT2A/2C antagonists (MER 810), SSRI/5HT1A partial agonists (vilazodone), NERI/5HT3 antagonists (MCI 225), melatonin agonists (agomelatine), SRI/5HT3 antagonists (litoxetine), SRI/5HT2A antagonists (LY628535, lubazodone, R-fluoxetine), acetylcholine releaser/NERI agents (bifemelane, teniloxazine), 5HT2A antagonists (MDL100907 and eplivanserin), and AChE inhibitor/SRI agents (RS-1259) to improve daytime fatigue associated with sleep related breathing disorders.

In yet another embodiment, alpha-1 agonists are used to reduce the sedating side effects associated with drugs that improve the hypoxic index, such as 5HT1A agonist/SRI/5HT2A/2C antagonists, SSRI/5HT1A partial agonists, NERI/5HT3 antagonists, 5HT2/5HT3 antagonist/alpha-2 antagonists, melatonin agonists, SRI/5HT3 antagonists, SRI/5HT2A antagonists, acetylcholine releaser/NERI agents, 5HT2A antagonists, and AChE inhibitor/SRI agents.

6. 5-HT1A Serotonin Receptor Agonists. In yet another embodiment, the sleep related breathing disorders are treated with a 5-HT1A serotonin receptor agonists (5-HT1A agonist). Useful 5-HT1A serotonin receptor agonists include any drug that binds to and activates the 5-HT1A receptor, including partial agonists. Preferred compounds include buspirone, gepirone, alnespirone, and Org 13011.

In a preferred embodiment, the 5-HT1a agonist is given in combination with a NERI or NSRI and/or an alpha-1 agonist.

An alternative approach to improving OSA is to directly utilize a 5-HT1a agonist, a compound class that appears to increase respiratory drive and may increase upper airway tone. This effect can be augmented by the addition of a noradrenergic reuptake inhibitor.

In another preferred embodiment, 5HT1A agonists may be used in combination with a 5HT2/5HT3 antagonist/alpha-2 antagonist to reduce the excessive daytime drowsiness associated with 5HT2/5HT3 antagonist/alpha-2 antagonist use for the treatment of sleep related breathing disorders.

7. 5-HT2A/2C Serotonin Receptor Agonists. In a further embodiment, the sleep related disorders are treated with a 5-HT2A/2C serotonin receptor agonist (5-HT2A/2C agonist). In a preferred embodiment, the 5-HT2A/2C agonist is given in combination with an NERI or NSRI and/or an alpha2 antagonist and/or an alpha1 agonist.

Direct stimulation of 5HT2A or 5HT2C receptors in the hypoglossal motor nucleus activates motor neurons controlling upper airway smooth muscle, and therefore agonists at these receptors are useful in OSA. Augmentation by enhancing noradrenergic tone to the hypoglossal motor nucleus improves the efficacy of 5-HT2A/2C agonists in OSA. Enhancement of noradrenergic tone can be accomplished by NERIs, NSRIs, alpha2 antagonists (which activate LC neurons to the hypoglossal nucleus), and by alpha1 agonists (which directly stimulate hypoglossal motor neurons).

Useful 5-HT2A/2C serotonin receptor agonists include any drug that binds to and activates the 5-HT2a and 5HT2C serotonin receptors. Included amonts the 5-HT2A/2C serotonin receptor antagonists is m-chlorophenylpiperazine (mcPP).

8. Melatonin Agonists. In yet another embodiment, the sleep related breathing disorders are treated with a melatonin agonist. In a preferred embodiment, the melatonin agonists are given in combination with a 5HT2B/2C antagonist. Melatonin agonists with 5HT2B/2C antagonist activity can also be used to treat OSA. Melatonin receptor stimulation re-sets circadian rhythms, reduce sleep latency and improve total sleep time while 5HT2B/2C blockade enhances SWS and increase respiratory drive. Agomelatine is a representative agent of this class.

Useful melatonin agonists include any drug that binds to and activates the melatonin receptor. Preferred compounds include melatonin and agomelatine

9. 5HT2B/2C Serotonin Receptor Antagonists. Useful 5HT2B/2C serotonin receptor antagonists (5HT2B/2C antagonists) include any drug that binds to but does not activate the 5HT2B or 5HT2C serotonin receptor subtypes. Agomelatine is an example of a compound with 5HT2B/2C antagonist activity.

10. Quinacrine and Quinacrine Derivatives. In a further embodiment, the sleep related breathing disorders are treated with quinacrine. In a preferred embodiment, the quinacrine is given in combination with a serotonergic agent, such as a 5HT1A agonist/SRV5HT2A/2C antagonist (MER 810), a compound with both SSRI and 5HT1A partial agonist activity, such as vilazodone, or a compound that acts as a NERI and a 5HT3 serotonin receptor antagonist, such as MC1225 (4-(2-Fluorophenyl)-6-methyl-2-(1-piperazinyl)thieno[2,3-D]pyrimidine monohydrate hydrochloride) to treat the sleep related breathing disorder. In another embodiment, quinacrine may be used in combination with a 5HT2/5HT3 antagonist/alpha-2 antagonist to reduce the excessive daytime drowsiness and weight gain associated with 5HT2/5HT3 antagonist/alpha-2 antagonist use for the treatment of sleep related breathing disorders.

As used herein, “a quinacrine” and “quinacrines”, refer to quinacrine and quinacrine derivatives, having the following general formula:

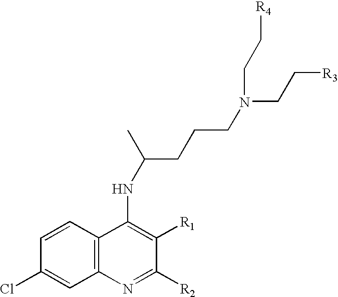

wherein R1 and R2 may be hydrogen, an alkyl group, a hydroxyl group, a methoxy group, a halogen group, an amino group, or together may form a substituted or unsubstituted fused aromatic ring. Preferred substituents of the aromatic ring include an alky group, a hydroxyl group, amino group, or methoxy group.

R3 and R4 may be hydrogen, a hydroxy group, alkyl group, methoxy group, or halogen group.

A preferred compound is quinacrine, wherein R1 and R2 together form a fused aromatic ring substituted with a methoxy group, and has the following chemical name: Quinacrine (6-chloro-9-(1-methyl-4-diethylamine)butylamino-2-methoxyacridine) is available as the dihydrochloride (quinacrine·hydrochloride, USP) in 100-mg tablets as a bright yellow, odorless, bitter crystalline powder that is water soluble (1:35) and 80% quinacrine base. Inactive ingredients include pharmaceutical glaze, starch, talc, and stearic acid.

Another preferred compound is chloroquine, shown below, wherein R1-R4 are hydrogen. Quinacrine differs from chloroquine only in having an acridine nucleus (an extra benzene ring) instead of a quinolone. Quinacrine has the chemical name 6-chloro-9-[[4-(diethylamino)-1-methylbutyl]amino]-2-methoxyacridine.

Yet another preferred compound is hydroxychloroquine, wherein R1-R3 are hydrogen, and R4 is a hydroxyl group.

11. Acetylcholinesterase (AChE) Inhibitors. In another embodiment, acetylcholinesterase inhibitors, such as donepezil, may be used to treat sleep related breathing disorders. In a preferred embodiment, the disorders are treated with acetylcholinesterase inhibitors in combination with serotonergic agents, such as a 5HT1A agonist/SRI/5HT2A/2C antagonist (MER810), a compound with both SSRI and 5HT1A partial agonist activity (vilazodone), or a compound that acts as a NERI and a 5HT3 serotonin receptor antagonist, such as MCI 225.

Useful acetylcholinesterase inhibitors include any compound that binds to and inhibits acetylcholinesterase. Preferred compounds include (±)-2,3-dihydro-5,6-dimethoxy-2-1[[1-(phenylmethyl)-4-piperidinyl]methyl]-1H-inden-1-one(donepezil), (4aS,6R,8aS)-4a,5,9,10,11,12-hexahydro-3-methoxy-11-methyl-6H-benzofuro[3a,3,2-ef][2]benzazepin-6-ol(galantamine), tetrahydroaminoacridine (THA, or tacrine), (S)—N-ethyl-N-methyl-3-[1-(dimethylamino)ethyl]-phenylcarbamate hydrogen(2R,3R)-tartrate(rivastigmine).

12. Cannabinoid Agonists. In yet another embodiment, cannabinoid agonists, such as THC and CP55940, may be used to treat sleep related breathing disorders. In a preferred embodiment, the disorders are treated with cannabinoid agonists (such as THC and CP55940) in combination with serotonergic agents, such as a 5HT1A agonist/SRI/5HT2A/2C antagonist (MER810), a compound with both SSRI and 5HT1A partial agonist activity (vilazodone), or a compound that acts as a NERI and a 5HT3 serotonin receptor antagonist, such as MCI 225.

Useful cannabinoid agonists include any compound that binds to and activates cannabinoid receptors. Preferred compounds include Delta-9-tetrahydrocannibinol (Delta-9-THC), Delta-8-tetrahydrocannibinol (Delta-8-THC), (−)-cis-3-[2-hydroxy-4-(1,1-dimethylheptyl)phenyl]-trans-4-(3-hydroxypropyl)cyclohexanol (CP 55940), 11-hydroxy-delta-8-THC-dimethylheptyl(HU-210), R(+)-[2,3-dihydro-5-methyl-3-[(morpholinyl)methyl]pyrrolo[1,2,3-de]-1,4-benzoxazinyl]-(1-naphthalenyl)methanone mesylate (WIN55212-2), 3-(5′-cyano-1′,1′-dimethylpentyl)-

1-(4-N-morpholinobutyryloxy)-delta-8-THC hydrochloride(0-1057), anandamide, and methanandamide, 2-arachidonyl glycerol (2-AG), arachidonyl-2′-chloroethylamide (ACEA) and arachidonylcyclopropylamide (ACPA).

13. Drugs with SSRI and 5HT1A Serotonin Receptor Partial Agonist Activity (SSRI/5HT1A Partial Agonists). In still a further embodiment, a compound with both SSRI and 5HT1A partial agonist activity, such as vilazodone may be given in combination with a compound that acts as a NERI and a 5HT3 serotonin receptor antagonist, such as MCI 225 for the treatment of sleep related breathing disorders.

Useful drugs with SSRI and 5HT1A serotonin receptor partial agonist activity include any drug that acts as a selective serotonin reuptake inhibitor and as a partial agonist at the 5HT1A receptor. Preferred drugs include 5-{4-[4-(5-Cyano-3-indolyl)-butyl)-butyl]-1-piperazinyl}-benzofuran-2-carboxamide(vilazodone).

14. Drugs with NERI and 5HT3 Serotonin Receptor Antagonist Activity. Useful drugs with NERI and 5HT3 serotonin receptor antagonist activity (NERI/5HT3 antagonists) include any drug that acts as a norepinephrine reuptake inhibitor (NERI) and as an antagonist at the 5HT3 serotonin receptor. Preferred drugs include MC1225 (4-(2-fluorophenyl)-6-methyl-2-(1-piperazinyl)thieno[2,3-D]pyrimidine monohydrate hydrochloride).

15. Drugs with Serotonin Reuptake Inhibitor and 5HT3 Serotonin Receptor Antagonist Activity. In another embodiment, the sleep related breathing disorders are treated with any drug having serotonin reuptake inhibitor (SRI) activity and 5HT3 serotonin receptor antagonist activity (SRI/5HT3 antagonist). Preferred drugs in this category include litoxetine (4-(2-naphtylmethoxy)piperidine).

In a preferred embodiment, the SRI/5HT3 serotonin receptor antagonists are given in combination with 5HT2A antagonists (MDL100907), alpha-1 agonists, alpha-2 antagonists, NERIs, NSRIs, MAO-B inhibitors (deprenyl), AChE inhibitors, or cannabinoid agonists in the treatment of sleep apnea/sleep disordered breathing.

16. Drugs with Serotonin Reuptake Inhibitor and 5HT2A Serotonin Receptor Antagonist Activity. In another embodiment, the sleep related breathing disorders are treated with any drug having serotonin reuptake inhibitor (SRI) activity and 5HT2A serotonin receptor antagonist activity (SRI/5HT2A antagonist). Preferred drugs in this category include LY628535, lubazodone ((S)-2-[[(7-fluoro-4-indanyl)oxy]methyl]morpholine hydrochloride), and R-fluoxetine (±)-N-methyl-3-phenyl-3-((α,α,α-trifluoro-P-tolyl)oxy)propylamine hydrochloride.

In a preferred embodiment, the SRI/5HT2A serotonin receptor antagonists are given in combination with alpha-1 agonists, alpha-2 antagonists, NERI, NSRIs, MAO-B inhibitors (deprenyl), AChE inhibitors, cannabinoid agonists, or 5HT3 antagonists (ondansetron) in the treatment of sleep apnea/sleep disordered breathing.

17. Drugs with Acetylcholine Releaser and Norepinephrine Reuptake Inhibitor Activity. In another embodiment, the sleep related breathing disorders are treated with any drug that acts as an acetylcholine releaser and as an norepinephrine reuptake inhibitor (Acetylcholine Releaser/NERI agent). Preferred drugs in this category include bifemelane (4-(o-benzylphenoxy)-N-methylbutylamine(hydrochloride)) and teniloxazine ((±)-2-[[(α-2-thienyl-o-tolyl)oxy]methyl]morpholine(Z)-2-butenedioate).

In a preferred embodiment, the acetylcholine releaser/NERI agents are given in combination with alpha-1 agonists, alpha-2 antagonists, SSRIs, MAO-B inhibitors (deprenyl), cannabinoid agonists, 5HT3 antagonists (ondansetron), 5HT2A antagonists, SRI/5HT3 antagonists, or SRI/5HT2A antagonists in the treatment of sleep apnea/sleep disordered breathing.

18. 5HT3 Serotonin Receptor Antagonists. In another embodiment, the sleep related breathing disorders are treated with any drug having 5HT3 serotonin receptor antagonist activity (5HT3 antagonists) in combination with SSRIs, alpha-1 agonists, alpha-2 antagonists, NERIs, NSRIs, MAO-B inhibitors (deprenyl), AChE inhibitors, or cannabinoid agonists. Preferred 5HT3 antagonists include ondansetron ((±) 1,2,3,9-tetrahydro-9-methyl-3-[(2-methyl-1H-imidazol-1-yl) methyl]-4H-carbazol-4-one monohydrochloride, dihydrate), dolasetron ((2α, 6α, 8α, 9αβ)-octahydro-3-oxo-2,6-methano-2H-quinolizin-8-yl-1H-indole-3-carboxylate monomethanesulfonate, monohydrate), granisetron (endo-N-(9-methyl-9-azabicyclo[3.3.1]non-3-yl)-1-methyl-1H-indazole-3-carboxamide hydrochloride), tropisetron (3-tropanyl-indole-3-carboxylate hydrochloride), ramosetron (5-[(1-methyl-3-indolyl) carboxyl]-4,5,6,7-tetrahydro-1H-benzimidazol hydrochloride, and palonosetron ((3αS)-2-[(S)-1-Azabicyclo[2.2.2]oct-3-yl]-2,3,3α,4,5,6-hexahydro-1-oxo 1Hbenz[de]isoquinoline hydrochloride).

19. 5HT2A Serotonin Receptor Antagonists. In another embodiment, the sleep related breathing disorders are treated with any drug having 5HT2A serotonin receptor antagonist activity (5HT2A antagonists) in combination with 5HT3 antagonists, SSRIs, alpha-1 agonists, alpha-2 antagonists, NERIs, NSRIs, MAO-B inhibitors (deprenyl), AChE inhibitors, or cannabinoid agonists. Preferred 5HT2A antagonists include MDL 100907 (R-(+)-α-(2,3-dimethyoxyphenyl)-1-(2-(4-fluorophenyl)ethyl)-4-piperidine-methanol), EMR-62218, and eplivanserin (Z) or (E)-2 butendedionate salt of O-(2-dimethylamino)ethyl) oxime-1-(2-fluorophenyl)-3-(4-hydroxyphenyl)-1-propanone.(2:1). Other examples of 5HT2A antagonists include ACP-102 and APD-125, which are 5HT2A inverse agonists, but serve as functional 5HT2A antagonists in vivo.

20. Monoamine Oxidase (MAO)-B Inhibitors. Useful MAO-B inhibitors include any drug that inhibits the activity of monoamine oxidase-B. Preferred compounds include: L-deprenyl ((R)-(−)-N,α-Dimethyl-N-(2-propynyl)phenethylamine hydrochloride)(selegiline); Ro16-6491 (N-(2-aminoethyl)-4-chlorobenzamide); pargyline (N-Methyl-N-propargylbenzylamine); lazabemide (N-(2-aminoethyl)-5-chloro-2-pyridine carboxamide hydrochloride); and mofegiline ((E)-2-fluoromethylene)-4-(p-fluorophenyl)butylamine hydrochloride).

21. Drugs with Acetylcholinesterase Inhibitor and Serotonin Reuptake Inhibitor Activity. In yet another embodiment, the sleep related breathing disorders are treated with any drug that acts as an inhibitor of acetylcholinesterase and as a serotonin reuptake inhibitor (AChE Inhibitor/SRI agent). Preferred compounds include RS-1259 ((4-[(1S)-methylamino-3-(4-nitrophenoxy)]propylphenyl N,N-dimethylcarbamate(fumaric acid)(½) salt).

In a preferred embodiment, the AChE inhibitor/SRI may also be used to treat the disorders in combination with NERIs, NSRIs, alpha-1 agonists, alpha-2 antagonists, MAO-B inhibitors, 5HT2A antagonists, 5HT3 antagonists, or cannabinoid agonists.

22. Drugs with 5HT2/5HT3 Serotonin Receptor Antagonist and Alpha-2 Adrenergic Receptor Antagonist Activity. In a further embodiment, the sleep related breathing disorders are treated with drugs that act as antagonists at both the 5HT2 and 5HT3 serotonin receptors and at alpha-2 adrenergic receptors (5HT2/5HT3 antagonist/alpha-2 antagonists). Preferred compounds include mirtazapine (1,2,3,4,10,14b-hexahydro-2-methylpyrazino[2,1-a]pyrido[2,3-c]benzazepine.

In some embodiments, mirtazapine is used as a monotherapy for the treatment of sleep related breathing disorders. In some embodiments, mirtazapine is administered at a dosage as low as about 5 mg per day. In further embodiments, mirtazapine is administered at a dosage of less than about 35 mg per day. In particular embodiments, mirtazapine is administered as a monotherapy at a dosage of about 7.5 to about 30 mg per day.

In a some embodiments, a 5HT2/5HT3 antagonist/alpha-2 antagonist is given in combination with AChE inhibitors, SSRIs, NERIs, NSRIs, alpha-1 agonists, AChE inhibitors/SRI agent, acetylcholine releaser/NERI agents, MAO-B inhibitors, or cannabinoid agonists to treat the disorder. In other embodiments, a 5HT2/5HT3 antagonist/alpha-2 antagonist, such as mirtazapine, is given in combination with zonisamide as described in more detail below. In some embodiments, zonisamide is administered at a dose of about 5 mg to about 500 mg, in particular about 10 mg to about 300 mg per day, when co-administered along with mirtazapine. In particular embodiments, about 5 to about 500 mg of zonisamide are co-administered along with about 5 mg to about 35 mg of mirtazapine. In specific embodiments, about 10 to about 300 mg of zonisamide are administered along with about 7.5 to about 30 mg of mirtazapine per day. In more specific embodiments, about 100 mg of zonisamide are co-administered with about 7.5 to about 30 mg of mirtazapine per day.

23. Ergot Alkaloids. In another embodiment, the sleep related breathing disorders are treated with ergot alkaloids with serotonin receptor agonist activity. Preferred ergot alkaloids act as agonists at the 5HT1A, 5HT1D, 5HT2A serotonin receptors. In general, ergot alkaloids can be classified according to their different chemical structures, for example, ergolines, lysergic acid derivatives, ergot peptide alkaloids and dihydrogenated ergot peptide alkaloids. Many ergot alkaloids and their derivatives are known. See for example, U.S. Pat. No. 3,896,228 to Richardson, U.S. Pat. No. 3,987,173 to Borredon, U.S. Pat. No. 4,229,451 to Fehr et al., U.S. Pat. No. 4,315,937 to Maclay et al., U.S. Pat. No. 4,366,145 to Stoopak et al, U.S. Pat. No. 4,440,722 to Djorjevic, U.S. Pat. No. 4,462,983 to Azria et al, and U.S. Pat. No. 6,037,346 to Doherty et al. Preferred compounds include ergotamine, dihydroergotamine, acetergamine, brazergoline, bromerguride, cianergoline, delorgotrile, disulergine, ergonovine maleate, etisulergine, lergotrile, lysergide, mesulergine, metergoline, metergotamine, nicergoline, pergolide, propisergide, proterguride, terguride.

24. Norepinephrine/Dopamine Reuptake Inhibitors. In yet another embodiment, drugs that inhibit the reuptake of norepinephrine and dopamine (NERI/DRI compounds) may be used in combination with 5HT2/5HT3 antagonist/alpha-2 antagonists, such as mirtazapine, to reduce excessive daytime drowsiness and weight gain associated with the use of 5HT2/5HT3 antagonist/alpha-2 antagonists in the treatment of sleep related breathing disorders.

Examples of NERI/DRI compounds that may be used include bupropion ((±)-1-(3-chlorophenyl)-2-[(1,1-dimethylethyl)amino]-1-propanone hydrochloride).

25. Dopamine D1 Receptor Agonists. Alternatively, dopamine D1 receptor agonists (D1 agonists) may be used in combination with a 5HT2/5HT3 antagonist/alpha-2 antagonist to reduce the excessive daytime drowsiness associated with 5HT2/5HT3 antagonist/alpha-2 antagonist use for the treatment of sleep related breathing disorders. Useful D1 agonists include any compound that binds and activates the D1 dopamine receptor. Preferred D1 agonists include: SKF38393 (1-Phenyl-2,3,4,5-tetrahydro-(1H)-3-benzazepine-7,8-diol); CY-208-243 ((−)-(6aR,12bR)-4,6,6a,7,8,12b-Hexahydro-7-methylindolo[4,3-a]phenanthridine); dihydrexidine (trans-10,11-dihydroxy-5,6,6a,7,8,12b-hexahydrobenzo[a]phenthnidine); SKF82958 (6-Chloro-7,8-dihydroxy-3-allyl-1-phenyl-2,3,4,5-tetrahydro-1H-3-benzazepine); A77636 ((1R-cis)-1-(Aminomethyl)-3,4-dihydro-3-tricyclo[3.3.1.13,7]dec-1-yl-[1H]-2-benzopyran-5,6-diol hydrochloride); and A68930 (cis-(±)-(Aminomethyl)-3,4-dihydro-3-phenyl-1H-2-benzopyran-5,6-diol hydrochloride).

26. Carbonic Anhydrase Inhibitors. In a further embodiment, carbonic anhydrase inhibitors may be used to treat sleep related breathing disorders either alone or in combination with drugs selected from the group consisting of 5HT1A agonist/SRI/5HT2A/2C antagonists, selective serotonin reuptake inhibitors (SSRIs), norepinephrine and serotonin reuptake inhibitors (NSRIs), selective norepinephrine reuptake inhibitors (NERIs), dopamine/serotonin receptor antagonists, NERI/5HT3 antagonists, SRI/5HT3 antagonists, and SRI/5HT2A antagonists.

In yet a further embodiment, carbonic anhydrase inhibitors may be used to in combination with a 5HT2/5HT3 antagonist/alpha-2 antagonist to reduce the weight gain associated with 5HT2/5HT3 antagonist/alpha-2 antagonist use for the treatment of sleep related breathing disorders. Useful carbonic anhydrase inhibitors include any compound that inhibits the carbonic anhydrase enzyme. Preferred compounds include acetazolamide (N-(5-[Aminosulfonyl]-1,3,4-thiadiazol-2-yl)acetamide)), zonisamide (1,2-Benzisoxazole-3-methanesulfonamide), methazolamide (N-(4-Methyl-2-sulfamoyl-Δ2-1,3,4-thiadiazolin-5-ylidene) acetamide), dichlorphenamide, and topiramate (2,3:4,5-bis-O-(1-methylethylidene)-36-D-fructo-pyranose sulfamate).

Both acetazolamide and zonisamide not only reduce appetite (and can counteract the pro-appetite effects of 5HT2/5HT3 antagonist/alpha-2 antagonists), but also enhance respiratory drive by producing metabolic acidosis via inhibition of carbonic anhydrase.

27. Corticosteroids. In yet another embodiment, corticosteroids may be used in combination with quinacrine, acetylcholinesterase inhibitors, cannabinoid agonists, 5HT1A agonist/SRI/5HT2A/2C antagonists, alpha-2 adrenergic receptor antagonists, 5-HT1A serotonin receptor agonists, 5HT2A/2C serotonin receptor agonists, melatonin agonists, 5HT3 serotonin receptor antagonists, 5HT2A serotonin receptor antagonists, selective serotonin reuptake inhibitors, norepinephrine reuptake inhibitors, norepinephrine/serotonin reuptake inhibitors, alpha-1 adrenergic receptor agonists, monoamine oxidase (MAO)-B inhibitors, ergot alkaloids, dopamine D1 receptor agonists, carbonic anhydrase inhibitors, 5HT2B/2C serotonin receptor antagonists, serotonin reuptake inhibitor/5HT3 serotonin receptor antagonists, serotonin reuptake inhibitor/5HT2A serotonin receptor antagonists, acetylcholine releaser/norepinephrine reuptake inhibitor agents, norepinephrine reuptake inhibitor/5-HT3 serotonin receptor antagonists, selective serotonin reuptake inhibitor/5HT1A serotonin receptor partial agonists, acetylcholinesterase inhibitor/serotonin reuptake inhibitor agents, 5HT2 antagonist/alpha-1 agonists, 5HT2/5HT3 serotonin receptor antagonist/alpha-2 adrenergic receptor antagonists, or norepinephrine reuptake inhibitor/dopamine reuptake inhibitor compounds for the treatment of sleep related breathing disorders. The compounds are preferably delivered nasally.

In a preferred embodiment the corticosteroid is combined with a 5HT2/5HT3 serotonin receptor antagonist/alpha-2 adrenergic receptor antagonist and delivered nasally. Useful corticosteroids include hydrocortisone (11β,17α,21-Trihydroxypregn-4-ene-3,20-dione), cortisone (17α,21-Dihydroxy-4-pregnene-3,11,20-trione), dexamethasone (9α-Fluoro-16a-methyl-11β,17α,21-trihydroxy-1,4-pregnadiene-3,20-dione) and prednisone (17α,21-Dihydroxy-1,4-pregnadiene-3,11,20-trione).

28. Dopamine-Releasing Compounds. In a further embodiment, dopamine-releasing compounds may be used in combination with 5HT2/5HT3 antagonist /alpha2 antagonists to reduce the excessive daytime sleepiness and/or weight gain associated with the use of 5HT2/5HT3 antagonist/alpha-2 antagonists in the treatment of sleep related breathing disorders. Useful dopamine-releasing compounds include any compound that induces release of dopamine from pre-synaptic dopaminergic neurons. Preferred compound include amantadine (1-aminoadamantane hydrochloride), rimantadine (α-methyltricyclo-(3.3.1.13,7)decane-1-methanamine hydrochloride, amphetamines, such as methamphetamine (S)—N, (alpha)-dimethylbenzeneethanamine hydrochloride), dextroamphetamine (d-α-methylphenethylamine), and laevoamphetamine, and methylphenidate (methyl α-phenyl-2-piperidineeacetate hydrochloride).

29. Dopamine and Serotonin Receptor Antagonists. In another embodiment, drugs that act as both dopamine receptor antagonists and serotonin receptor antagonists (dopamine/serotonin receptor antagonists) may be used in combination with a carbonic anhydrase inhibitor for the treatment of sleep related breathing disorders. Preferred compounds include those which act as antagonists at D2 dopamine receptors and 5HT2 serotonin receptors. Useful dopamine/serotonin receptor antagonists include clozapine (8-chloro-1-(4-methyl-1-piperazinyl)-5H-dibenzo[b,e][1,4]diazepine), ziprasidone ([2-[4-(1,2-benzisothiazol-3-yl)-1-piperazinyl]ethyl]-6-chloro-1,3-dihydro-2H -indol-2-one), risperidone (3-[2-[4-(6-fluoro-1,2-benzisoxazol-3-yl)-1-piperidinyl]ethyl]-6,7,8,9-tetrahydro-2-methyl-4H-pyrido[2,2-a]pyrimidin-4-one), olanzapine (2-methyl-4-(4-methyl-1-piperazinyl)-10H-thieno[2,3-b][1,5]benzodiazepine), quetiapine (2-[2-(4-dibenzo [b,f][1,4]thiazepin-11-yl-1-piperazinyl)ethoxy]-ethanol fumarate), sertindole (1-[2-[4-[5 -chloro-1-(4-fluorophenyl)-1H-indol-3-yl]-1-piperidinyl]ethyl]-2-imidazolidinone), and zotepine (2-chloro-11-(2-dimethyl-aminoethoxy)dibenzo[b,f]thiepine).

30. H1 Histamine Receptor and 5HT2 Serotonin Receptor Antagonists. In a further embodiment, drugs that act as H1 histamine receptor antagonists and 5HT2 serotonin receptor antagonists (H1/5HT2 antagonist) may be used to treat the sleep related breathing disorders, either alone, or in combination with drugs selected from the group consisting of SSRIs, NSRIs, NERIs, carbonic anhydrase inhibitors, SSRI/5HT1A partial agonists, and SRI/5HT3 antagonists.

A preferred H1/5HT2 antagonist is cyproheptadine (4-(5H-dibenzo[a,d]cyclohepten-5-ylidene)-1-methylpiperidine hydrochloride). This compound contains sleep-promoting properties, including increasing slow wave sleep and reducing REM sleep, which may provide benefits in the treatment of obstructive sleep apnea (OSA) and other sleep related breathing disorders.

B. Compounds Used to Reduce Pressure Applied During Continuous Positive Airway Pressure (CPAP) Therapy. In some embodiments, the pressure applied during continuous positive airway pressure (CPAP) therapy in the treatment of sleep related breathing disorders may be reduced by administration of an effective amount of one or more compounds that suppress REM sleep, increase deep slow wave sleep, and increase the tone of upper airway muscles during sleep and/or increase respiratory drive, in concert with CPAP. Using these drugs in combination with CPAP can reduce the airflow requirements of CPAP and make the CPAP procedure more tolerable to patients. Preferred drugs include quinacrine, acetylcholinesterase inhibitors, cannabinoid agonists, 5HT1A agonist/SRI/5HT2A/2C antagonists, alpha-2 adrenergic receptor antagonists, 5-HT1A serotonin receptor agonists, 5HT2A/2C serotonin receptor agonists, melatonin agonists, 5HT3 serotonin receptor antagonists, 5HT2A serotonin receptor antagonists, selective serotonin reuptake inhibitors, norepinephrine reuptake inhibitors, norepinephrine/serotonin reuptake inhibitors, alpha-1 adrenergic receptor agonists, monoamine oxidase (MAO)-B inhibitors, ergot alkaloids, 5HT2B/2C serotonin receptor antagonists, serotonin reuptake inhibitor/5HT3 serotonin receptor antagonists, serotonin reuptake inhibitor/5HT2A serotonin receptor antagonists, acetylcholine releaser/norepinephrine reuptake inhibitor agents, norepinephrine reuptake inhibitor/5-HT3 serotonin receptor antagonists, selective serotonin reuptake inhibitor/5HT1A serotonin receptor partial agonists, acetylcholinesterase inhibitor/serotonin reuptake inhibitor agents, 5HT2 antagonist/alpha-1 agonists, 5HT2/5HT3 serotonin receptor antagonist/alpha-2 adrenergic receptor antagonists, and norepinephrine reuptake inhibitor/dopamine reuptake inhibitor compounds.

Preferred drug combinations include, 5HT2/5HT3 antagonist/alpha-2 antagonists and NERI/DRI compounds, 5HT3 antagonists and 5HT2A antagonists, 5HT3 antagonists and SRI/5HT2A antagonists, 5HT3 antagonists and 5HT2A/2C antagonist/SRI/5HT1A agonists, and 5HT3 antagonists and 5HT2 antagonist/alpha-1 agonists.

C. Salts and Derivatives. Although described above with reference specific to compounds, one can also utilize enantiomers, stereoisomers, metabolites, derivates and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418).

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a mammalian subject, cleaves to form a free hydroxyl or free amino, respectively. Examples of prodrugs include, but are not limited to, acetate, formate and benzoate derivatives of alcohol and amine functional groups.

A metabolite of the above-mentioned compounds results from biochemical processes by which living cells interact with the active parent drug or other formulas or compounds of the present invention in vivo. Metabolites include products or intermediates from any metabolic pathway.

D. Formulations. The compounds, or pharmaceutically acceptable salts thereof, including their polymorphic variations, can be formulated as pharmaceutical compositions. Such compositions can be administered orally, buccally, intravenously, parenterally, by inhalation spray, rectally, intradermally, transdermally, pulmonary, nasally or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. In the preferred embodiment the composition is administered orally.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The active compounds (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

Nasal formulations of quinacrine, acetylcholinesterase inhibitors, cannabinoid agonists, 5HT1A agonist/SRI/5HT2A/2C antagonists, alpha-2 adrenergic receptor antagonists, 5-HT1A serotonin receptor agonists, 5HT2A/2C serotonin receptor agonists, melatonin agonists, 5HT3 serotonin receptor antagonists, 5HT2A serotonin receptor antagonists, selective serotonin reuptake inhibitors, norepinephrine reuptake inhibitors, norepinephrine/serotonin reuptake inhibitors, alpha-1 adrenergic receptor agonists, monoamine oxidase (MAO)-B inhibitors, ergot alkaloids, dopamine D1 receptor agonists, carbonic anhydrase inhibitors, 5HT2B/2C serotonin receptor antagonists, serotonin reuptake inhibitor/5HT3 serotonin receptor antagonists, serotonin reuptake inhibitor/5HT2A serotonin receptor antagonists, acetylcholine releaser/norepinephrine reuptake inhibitor agents, norepinephrine reuptake inhibitor/5-HT3 serotonin receptor antagonists, selective serotonin reuptake inhibitor/5HT1A serotonin receptor partial agonists, acetylcholinesterase inhibitor/serotonin reuptake inhibitor agents, 5HT2 antagonist/alpha-1 agonists, 5HT2/5HT3 serotonin receptor antagonist/alpha-2 adrenergic receptor antagonists, or norepinephrine reuptake inhibitor/dopamine reuptake inhibitor compounds are used in combination with nasal formulations of corticosteroids, such as hydrocortisone, cortisone, dexamethasone and prednisone, for the treatment of sleep related breathing disorders. In a preferred embodiment, a 5HT2/5HT3 serotonin receptor antagonist/alpha-2 adrenergic receptor antagonist, such as mirtazapine, is combined with a corticosteroid. Typically, patients complaining of snoring/apnea/SDB are first prescribed nasal corticosteroids to help reduce upper airway swelling. Nasal formulations of the compounds listed above combined with corticosteroids should vastly improve the symptoms of sleep related breathing disorders.

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

In addition, combinations of immediate release compositions and delayed release/extended release compositions may be formulated together.

In one embodiment, formulations of the combination of a NERI/DRI compound, quinacrine, NERI, NSRI, dopamine D1 agonist, 5HT1A agonist, carbonic anhydrase inhibitor, or dopamine releasing compound with a 5HT2/5HT3 antagonist/alpha-2 antagonist, such as mirtazapine, which allow for immediate release of the 5HT2/5HT3 antagonist/alpha-2 antagonist and delayed release of the quinacrine, NERI/DRI compound, NERI, NSRI, dopamine D1 agonist, 5HT1A agonist, carbonic anhydrase inhibitor, or dopamine-releasing compound are provided. The combination may be administered before bed time which requires immediate release of mirtazapine for adequate treatment of sleep related breathing disorders throughout the night. Delayed release of the NERI/DRI compound, quinacrine, NERI, NSRI, dopamine D1 agonist, 5HT1A agonist, carbonic anhydrase inhibitor, or dopamine-releasing compound is important so that adequate concentrations are available in the circulation following sleep to counteract the excessive daytime sleepiness and/or increased appetite/weight gain associated with 5HT2/5HT3 antagonist/alpha-2 antagonist use.

II. Disorders to be Treated. Set forth below are some sleep related breathing disorders that are treated by methods according to the present invention.

A. Sleep Apnea. In general sleep apnea is defined as an intermittent cessation of airflow at the nose and mouth during sleep. By convention, apneas of at least 10 seconds in duration have been considered important, but in most individuals the apneas are 20-30 seconds in duration and may be as long as 2-3 minutes. While there is some uncertainty as to the minimum number of apneas that should be considered clinically important, by the time most individuals come to attention of the medical community they have at least 10 to 15 events per hour of sleep.

Sleep apneas have been classified into three types: obstructive, central, and mixed.

1. Obstructive Sleep Apnea. Obstructive sleep apnea syndrome (OSAS) has been identified in as many as 24% of working adult men and 9% of similar women, with peak prevalence in the sixth decade. Habitual heavy snoring, which is an almost invariant feature of OSAS, has been described in up to 24% of middle aged men, and 14% of similarly aged women, with even greater prevalence in older subjects.

Obstructive sleep apnea syndrome's definitive event is the occlusion of the upper airway, frequently at the level of the oropharynx. The resultant apnea generally leads to a progressive-type asphyxia until the individual is briefly aroused from the sleeping state, thereby restoring airway patency and thus restoring airflow.

An important factor that leads to the collapse of the upper airway in OSAS is the generation of a critical subatmospheric pressure during the act of inspiration that exceeds the ability of the airway dilator and abductor muscles to maintain airway stability. Sleep plays a crucial role by reducing the activity of the muscles of the upper airways including the dilator and abductor muscles.

In most individuals with OSAS the patency of the airway is also compromised structurally and is therefore predisposed to occlusion. In a minority of individuals the structural compromise is usually due to obvious anatomic abnormalities, i.e, adenotonsillar hypertrophy, retrognathia, or macroglossia. However, in the majority of individuals predisposed to OSAS, the structural abnormality is simply a subtle reduction in airway size, i.e., “pharyngeal crowding.” Obesity also frequently contributes to the reduction in size seen in the upper airways. The act of snoring, which is actually a high-frequency vibration of the palatal and pharyngeal soft tissues that results from the decrease in the size of the upper airway lumen, usually aggravates the narrowing via the production of edema in the soft tissues.

The recurrent episodes of nocturnal asphyxia and of arousal from sleep that characterize OSAS lead to a series of secondary physiologic events, which in turn give rise to the clinical complications of the syndrome. The most common manifestations are neuropsychiatric and behavioral disturbances that are thought to arise from the fragmentation of sleep and loss of slow-wave sleep induced by the recurrent arousal responses. Nocturnal cerebral hypoxia also may play an important role. The most pervasive manifestation is excessive daytime sleepiness. OSAS is now recognized as a leading cause of daytime sleepiness and has been implicated as an important risk factor for such problems as motor vehicle accidents. Other related symptoms include intellectual impairment, memory loss, personality disturbances, and impotence.

The other major manifestations are cardiorespiratory in nature and are thought to arise from the recurrent episodes of nocturnal asphyxia. Most individuals demonstrate a cyclical slowing of the heart during the apneas to 30 to 50 beats per minute, followed by tachycardia of 90 to 120 beats per minute during the ventilatory phase. A small number of individuals develop severe bradycardia with asystoles of 8 to 12 seconds in duration or dangerous tachyarrhythmias, including unsustained ventricular tachycardia. OSAS also aggravates left ventricular failure in patients with underlying heart disease. This complication is most likely due to the combined effects of increased left ventricular afterload during each obstructive event, secondary to increased negative intrathoracic pressure, recurrent nocturnal hypoxemia, and chronically elevated sympathoadrenal activity.

2. Central Sleep Apnea. Central sleep apnea is less prevalent as a syndrome than OSAS, but can be identified in a wide spectrum of patients with medical, neurological, and/or neuromuscular disorders associated with diurnal alveolar hypoventilation or periodic breathing. The definitive event in central sleep apnea is transient abolition of central drive to the ventilatory muscles. The resulting apnea leads to a primary sequence of events similar to those of OSAS. Several underlying mechanisms can result in cessation of respiratory drive during sleep. First are defects in the metabolic respiratory control system and respiratory neuromuscular apparatus. Other central sleep apnea disorders arise from transient instabilities in an otherwise intact respiratory control system.

Many healthy individuals demonstrate a small number of central apneas during sleep, particularly at sleep onset and in REM sleep. These apneas are not associated with any physiological or clinical disturbance. In individuals with clinically significant central sleep apnea, the primary sequence of events that characterize the disorder leads to prominent physiological and clinical consequences. In those individuals with central sleep apnea alveolar hypoventilation syndrome, daytime hypercapnia and hypoxemia are usually evident and the clinical picture is dominated by a history of recurrent respiratory failure, polycythemia, pulmonary hypertension, and right-sided heart failure. Complaints of sleeping poorly, morning headache, and daytime fatigue and sleepiness are also prominent. In contrast, in individuals whose central sleep apnea results from an instability in respiratory drive, the clinical picture is dominated by features related to sleep disturbance, including recurrent nocturnal awakenings, morning fatigue, and daytime sleepiness.

3. Mixed Sleep Apnea. An episode of mixed sleep apnea usually starts with a central component and then becomes obstructive in nature. Generally the central component of the apnea becomes less troublesome once the obstructive apnea is treated.

B. Other Sleep related Breathing Disorders. The invention further provides methods of treating other sleep related breathing disorders. Such breathing disorders are discussed in more detail hereafter.

1. Sleep Hypopnea. Hypopneas are episodes of shallow breathing during which airflow is decreased by at least 50%. They are usually accompanied by some degree of oxygen desaturation, which can be minor and transient. Like apnea, hypopnea is subdivided as being obstructive, central, or mixed. Obstructive hypopneas are episodes of partial upper airway obstruction. Respiratory efforts occur, but airflow is reduced. In central hypopnea, breathing effort and airflow are both decreased. Mixed hypopneas have both central and obstructive components. Individuals with OSA syndrome have pathologic degrees of obstructive apnea, obstructive hypopnea, or both.

2. Upper Airway Resistance Syndrome. The term Upper Airway Resistance Syndrome (UARS) is used to describe chronic daytime sleepiness in the absence of actual apneas or hypopneas, but often associated with snoring, and with brief, frequent arousals with an only slightly abnormal breathing pattern. Patients with the clinical features of apnea, hypopnea and nocturnal oxygen desaturation during polysomnography (PSG).

Patients with UARS lack the typical findings of apnea on PSG, and therefore, are often not diagnosed. The arousals and sleep fragmentation are related to an increased effort to breathe which can be diagnosed by measurement of pressure changes in the esophagus.

3. Snoring. It is estimated that over 50 million individuals snore nightly. The term “snoring” generally refers to a rough or hoarse sound that arises from a person's mouth during sleep. Snoring is believed to be generally caused by the narrowing of the pharyngeal airway such that turbulent airflow during relaxed breathing vibrates the soft parts of the pharyngeal passage, such as the soft palate, the posterior faucial pillars of the tonsils and the uvula. A restricted pharyngeal passageway can occur anatomically. For example, in children, this often is caused by obstruction due to enlarged tonsils or adenoids. In adults, it is not unusual for the narrowing to be caused by obesity. Further anatomical narrowing can be simple a matter of heredity, with some persons being predisposed towards a smaller pharyngeal cross-section. A reduced pharyngeal passageway may also be caused by a lack of muscle tone. Snoring may also be exacerbated by consuming either alcohol or drugs (such as tranquilizers, sleeping pills and antihistamines) prior to bedtime. Smoking can also contribute to the incidence of snoring since cigarettes may irritate the mucus membranes of the upper airway causing swelling and increased mucus production.

Snoring can indicate a more serious condition and, due to exhaustion resulting from lack of sleep, can cause other problems. For example, an association between snoring and coronary artery disease and hypertension has been found, and cardiac arrhythmia has been reported during sleep apnea attacks. As stated above, people with sleep apnea often snore, however, sleep apnea can also be present without snoring. Not only is the risk of cessation of breathing dangerous, lack of oxygen due to an obstructed pharyngeal passageway deprives the body of sufficient oxygen so that oxygen desaturation arises. Lack of oxygen may cause the brain to rouse the sleeper just enough to take a breath without fully awaking. This may occur hundreds of times a night, with the result that the snorer fails to get sufficient sleep. Moreover, being aroused from deep REM sleep on a repetitive basis may increase heart rate and blood pressure. Thus, snoring may increase the risk of heart attack and stroke (Leineweber et al. Sleep 27(7): 1344-1349 (2004)).

C. Conditions Associated with Sleep related Breathing Disorders. The present invention also provides for treatment of conditions that are associated with sleep related breathing disorders. Such conditions are concomitant to one or more sleep related breathing disorders.

1. Excessive Daytime Sleepiness and Weight Gain. Excessive daytime sleepiness frequently occurs in individuals suffering from sleep apnea. Its symptoms are an overwhelming desire to sleep during what should be waking hours, the need for frequent naps, the inability to concentrate, falling asleep during meetings, class, at work or driving. People find that excessive daytime sleepiness can interfere with their ability to be productive and maintain healthy social relationships. They sometimes feel low self-esteem, frustration, and anger at oneself caused by the disorder and are sometimes misunderstood as being lazy or unintelligent.

Obesity is also strongly linked to patients with sleep related breathing disorders, especially obstructive sleep apnea (OSA) (Vgontzas et al. Arch. Intern. Med. 154 (15): 1705-1711 (1994)). Patients with sleep apnea have difficulty losing weight and, in fact, are predisposed to excessive weight gain, far more than is evident in similarly obese control subjects proven to be free of OSA. Sleep related breathing disorders, including sleep-apnea contribute to weight gain by reducing an individual's physical activity level as a result of chronic sleepiness or fatigue.

In addition, drugs used to treat sleep related breathing disorders can also contribute to excessive daytime sleepiness and weight gain by causing sedation in a patient. The breathing disorder is effectively treated at night, however, a sedating drug with a long half-life can cause sleepiness or fatigue during the day.

D. Pressure in Continuous Positive Airway Pressure (CPAP) Therapy. A common treatment for sleep apnea is CPAP, or Continuous Positive Airway Pressure, therapy. A CPAP machine is usually about the size of a shoebox but may be smaller. A flexible tube connects the machine with a mask or other interface device that is worn over the nose and/or mouth. CPAP works by pushing air through the airway passage at a pressure high enough to prevent apneas and can be prescribed for both obstructive and central sleep apnea. The pressure is set according to the patient's sleep apnea.

Probably the most common complaint about CPAP is the pressure. The pressure settings for CPAP range from 2 centimeters H20 to 20 centimeters H20. The reference to “cm H2O” or “centimeter H2O” or “centimeters of water” or “cm water” refer to the pressure it takes to raise a narrow column of water a particular number of centimeters. In general, CPAP pressures range from about 2 to about 25, especially from about 2 to about 20 cm H2O. Patients with the higher pressures (above 12 centimeters) usually have more severe apnea and more severe daytime symptoms, especially excessive sleepiness. Some people experience nasal dryness, irritation, or congestion as a result of CPAP therapy. The higher a person's treatment pressure, the more likely nasal irritation will occur. In addition, some patients experience pressure sores.

III. Methods of Use. Methods of using compounds, compositions and dosage forms according to the invention are set forth in detail below.

A. Administration Protocol. The compositions are administered in a therapeutically effective dosage, which is generally considered to be a dosage effective to prevent, ameliorate or alleviate one or more sleep related breathing disorders, one or more symptoms of sleep related breathing disorders or one or more sequelae of sleep related breathing disorders. The compositions are generally administered orally, however other dosage regimes can be used, especially where the patient is unable or unwilling to take the composition orally. The compositions can be administered as immediate release, sustained release, intermittent release, and/or delayed release formulations. The composition can be administered in a single dose, an escalating dose, or administered at an elevated dosage which is then decreased to a lower dosage after a particular circulating blood concentration of the compound has been achieved.

The method comprises co-administration of mirtazapine and at least one other active pharmaceutical ingredient. In some embodiments, the method comprises co-administration of mirtazapine and at least one other active pharmaceutical ingredient that improves airflow. The improvement of airflow can be effected by increasing the upper airway muscle tone of the patient during sleep, stabilizing the patient's respiratory drive, or both. In some embodiments of the invention, a compound that provides the effects of increasing the upper airway muscle tone of the patient during sleep and stabilizing the patient's respiratory drive is zonisamide. In other embodiments of the invention, a compound that provides the effect of stabilizing the patient's respiratory drive is topiramate, amantadine, bupropion, modafinil, r-modafinil, a 5HT1A agonist, or SDZ-NVI-085.

In some embodiments, mirtazapine is combined with one or more compounds that treat one or more symptoms of sleep related breathing disorders, such as daytime sleepiness. For example, mirtazapine can be combined with modafinil, which is effective for the treatment of drowsiness. In some embodiments, mirtazapine and modafinil can be combined in a single dosage form, while in other embodiments they can be prepared in separate dosage forms, and can even be administered at different times in a 24 hour period. (The compound SDZ-NVI-085 can be used in place of modafinil due to its similar alertness-stimulating properties.) In other embodiments, mirtazapine and topiramate, amantadine or bupropion can be combined in a single dosage form, while in other embodiments they can be prepared in separate dosage forms, and can even be administered at different times in a 24 hour period. In another embodiment, administration of mirtazapine nocte can be combined with daytime administration of amphetamine or other compound suitable for treatment of drowsiness.

An intermittent administration protocol may be used where chronic administration is not desirable. The compound or formulation is administered in time blocks of several days with a defined minimum washout time between blocks. Intermittent administration occurs over a period of several weeks to months to achieve a significant improvement in the symptoms of sleep related breathing disorders. Also staggered administration can be used where tolerability is an issue. For example, a first therapeutic agent can be administered for a period of time (e.g. 1-2 weeks) before commencement of administration of the second pharmaceutical agent. In particular embodiments, therapy with mirtazapine can be started at a first time point and co-administration of mirtazapine with topiramate or mirtazapine with topiramate can be started at a second time point, such as one to two weeks after commencement of mirtazapine administration. In such cases, it is convenient to present mirtazapine and a second active ingredient (zonisamide or topiramate) in a kit. Such a kit conveniently contains separate daily doses of mirtazapine alone for the first 1-30 days (wherein mirtazapine is optionally pared with a placebo pill that is taken in place of zonisamide or topiramate), and mirtazapine plus zonisamide or topiramate for the remainder of the time period covered by the kit.

In an exemplary kit, the kit comprises a blister pack having separate compartments for each of 30 days. Each of the first 1-14 compartments corresponding to the first 1-14 days of therapy contains mirtazapine alone or paired with a placebo. Each of the remaining 1-29 compartments contains mirtazapine paired with either zonisamide or topiramate. In one particular example there are 7 compartments, corresponding to the first 7 days of therapy, containing mirtazapine alone or paired with a placebo; and there are 21 compartments comprising mirtazapine paired with either zonisamide or topiramate. In another particular example, there are 14 compartments, corresponding to the first 14 days of therapy, containing mirtazapine alone or paired with a placebo; and there are 14 compartments comprising mirtazapine paired with either zonisamide or topiramate. It will be recognized that the period covered by the kit can vary from about 10 to about 90 days, with the number of compartments included in the kit corresponding to the number of days covered thereby. The number of days of mirtazapine only administration can also be varied from 1 to 45, with 7 to 30 days being considered most advantageous.

One of skill in the art would be able to choose administration protocols and determine appropriate dosing regimes to treat symptoms of sleep related breathing disorders based on bioavailability and half-life of the compound to be administered. For many of the disclosed compounds, appropriate dosage ranges have been established to maximize circulating concentrations of the compound and minimize side-effects.

The compound can be administered for a specific duration to improve symptoms of a particular sleep related breathing disorder. A suitable endpoint can be where one symptom of the disorder is treated by administration of the compound and the treatment considered effective. In other situations, the treatment can be considered effective when more than one symptom is treated.

B. Effective Dosage Ranges. Appropriate dosages can be determined by one of skill in the art based on using routine experimentation and standard techniques utilizing dosages currently approved. Compounds in the disclosed drug classes are known in the art and can be initially administered at similar doses and titrated appropriately to treat symptoms of sleep related breathing disorders in a given patient. Intra-patient variability is known in the art depending on the severity of symptoms and dosages are commonly adjusted to exact a particular therapeutic effect in a particular patient.

Therapeutically effective amounts for use in humans can also be determined from animal models. For example, a dose for humans can be formulated to achieve a circulating concentration that has been found to be effective in animals. Effective amounts for use in humans can also be determined from human data for the compounds used to treat other disorders, for example, neurological disorders. The amount administered can be the same amount administered to treat other neurological disorders or can be an amount higher or lower than the amount administered to treat other neurological disorders.

The optimal concentration of the drug in each pharmaceutical formulation varies according to the formulation itself. Typically, the pharmaceutical formulation contains the drug at a concentration of about 0.1 to 90% by weight (such as about 1-20% or 1-10%). Appropriate dosages of the drug can readily be determined by those of ordinary skill in the art of medicine by assessing amelioration of the sleep related breathing disorder in the patient, and increasing the dosage and/or frequency of treatment as desired. The optimal amount of the drug may depend upon the mode of administration, the age and the body weight of the patient, and the condition of the patient. Typically, the drugs are administered at a dosage of 0.001 to 100 mg/kg of body weight of the patient; e.g., the drug is administered at a dosage of 0.01 mg to 10 mg/kg or 0.1 to 1.0 mg/kg. Preferred daily doses of drug are approximately 1 mg to 800 mg/day. Preferred daily doses of quinacrine are approximately 10 to 200 mg/day, and preferably 50-100 mg/day.

In some embodiments, the effective dose of mirtazapine is in the range of about 7.5 to about 50 mg per day and the effective dose of zonisamide in combination with mirtazapine is in the range of about 25 to about 200 mg per day.

In other embodiments, the effective dose of mirtazapine is in the range of about 7.5 to about 50 mg per day and the effective dose of modafinil is in the range of about 100 to 400 mg per day, e.g. about 200 mg per day. The dose of modafinil can be divided into two doses in the morning and at noon.

It is understood that the disclosed methods are not limited to the particular methodology, protocols, and reagents described, as these may vary. It is also to be understood that the terminology used herein is for the purpose of describing particular embodiments only, and is not intended to limit the scope of the present invention which is limited only by the appended claims.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

The present invention thus provides methods of treating or reducing the symptoms of sleep apnea or other sleep related breathing disorders in an individual. The methods comprise administering to the individual an effective amount of a composition that suppresses REM sleep, increases deep slow wave sleep, and increases the tone of upper airway muscles during sleep and/or increase respiratory drive. The composition contains one or more compounds. When the composition contains a single compound as the active pharmaceutical ingredient (API), the single compound is capable of suppressing REM sleep, increasing deep slow wave sleep, and increasing the tone of upper airway muscles during sleep and/or increase respiratory drive. When the composition contains two or more compounds as the API, the two compounds in cooperation are capable of suppressing REM sleep, increasing deep slow wave sleep, and increasing the tone of upper airway muscles during sleep and/or increase respiratory drive.

In some embodiments, the invention provides a method of improving patient tolerance of CPAP therapy. In particular, the present invention provides methods of lowering the CPAP pressure necessary to treat an individual undergoing CPAP therapy for sleep apnea. The method entails administering to the individual undergoing CPAP therapy an amount of the compound (or where two or more compounds are employed, the compounds) sufficient to reduce the pressure at which CPAP therapy is effective to treat sleep apnea. In this way, patient comfort is increased and patient tolerance for CPAP therapy is increased as well. In some embodiments, the invention provides a method for lowering the effective pressure for CPAP therapy by at least 1 cm of H2O, comprising administering to the individual a sufficient amount of a composition that suppresses REM sleep, increases deep slow wave sleep, and increases the tone of upper airway muscles during sleep and/or increase respiratory drive. In other embodiments, the invention provides a method for lowering the effective pressure for CPAP therapy by at least 2 cm of H2O, especially at least about 4 cm H2O. In other embodiments, the invention provides a method of lowering the effective pressure of CPAP therapy from a first pressure exceeding the pressure limits of a CPAP device to a second pressure within the pressure range achievable by the CPAP device. In some embodiments, the effective pressure is lowered from a pressure exceeding 25 cm H2O to a pressure less than 25 cm H2O. In other embodiments, the effective pressure is lowered from a pressure exceeding 20 cm H2O to a pressure less than about 20 cm H2O. In other embodiments, the method comprises lowering the effective pressure of a CPAP machine for an individual to below about 18 cm H2O, especially less than about 15 cm H2O, more especially less than about 12 cm H2O and even more particularly less than about 10 cm H2O. In some embodiments, the method provides nearly complete relief from obstructive sleep apnea such that the minimal CPAP pressure of 3 or 4 cm H2O may be used to effectively treat an individual suffering obstructive sleep apnea. It is thus an object of the invention to improve the comfort level of an individual undergoing CPAP therapy for obstructive sleep apnea. It is further an object of the invention to thereby improve patient compliance with CPAP therapy. It is further an object of the invention to improve the efficacy of CPAP therapy, in some cases lowering the effective pressure into the range of pressures usually available to CPAP devices (e.g. about 3 or 4 cm H2O to about 20 to 25 cm H2O), and in other cases simply lowering the effective pressure into a range that is tolerable for the individual undergoing CPAP therapy.

The composition of the invention may be either a single compound (active pharmaceutical agent or API) or a combination of compounds. In the context of this invention, a combination of compounds means that the compounds are administered as part of the same therapy, either as a single dosage form or divided amongst separate dosage forms. For example, a combination of drugs includes a mixture of two or more drugs in a single dosage form, such as a tablet, a capsule, a caplet, a solution, a suspension, an elixir, a sol, a gel cap, a powder, etc. As another example, a combination of drugs includes a first drug administered in a first dosage form at a first time and a second drug administered in a second dosage form at a second time that may be the same or different from the first time. Thus, the first dosage form may be a tablet, a capsule, a caplet, a solution, a suspension, an elixir, a sol, a gel cap, a powder, etc. and second dosage form may be a tablet, a capsule, a caplet, a solution, a suspension, an elixir, a sol, a gel cap, a powder, etc. In some embodiments, the first dosage form is a tablet and the second is selected from a tablet, a capsule, a caplet, a solution, a suspension, an elixir, a sol, a gel cap and a powder. In some embodiments, the first dosage form is a capsule and the second is selected from a tablet, a capsule, a caplet, a solution, a suspension, an elixir, a sol, a gel cap and a powder. In some embodiments, the first dosage form is a caplet and the second is selected from a tablet, a capsule, a caplet, a solution, a suspension, an elixir, a sol, a gel cap and a powder. In some embodiments, the first dosage form is a solution and the second is selected from a tablet, a capsule, a caplet, a solution, a suspension, an elixir, a sol, a gel cap and a powder. In some embodiments, the first dosage form is a suspension and the second is selected from a tablet, a capsule, a caplet, a solution, a suspension, an elixir, a sol, a gel cap and a powder. In some embodiments, the first dosage form is a tablet and the second is selected from a sol, a capsule, a caplet, a solution, a suspension, an elixir, a sol, a gel cap and a powder. In some embodiments, the first dosage form is a gel cap and the second is selected from a tablet, a capsule, a caplet, a solution, a suspension, an elixir, a sol, a gel cap and a powder. In some embodiments, the first dosage form is a powder and the second is selected from a tablet, a capsule, a caplet, a solution, a suspension, an elixir, a sol, a gel cap and a powder.

C. Specific Compounds to be Administered. The following compounds may be administered to produce a therapeutic effect. As mentioned above, such compounds may be administered alone or in combination with other drugs. In particular, it is contemplated that two or more of the drugs set forth below may be combined in a mixture, in a unit dosage form comprising two such compounds, or in a kit comprising two such compounds in separate dosage forms to be co-administered as part of the same therapeutic regime. In particular, compounds to be administered include: mirtazapine as the sole active pharmaceutical ingredient (API); a combination of mirtazapine and zonisamide; milnacipran as the sole API; and a combination of milnacipran and zonisamide. Other embodiments will become apparent to the person skilled in the art upon consideration of the following.

1. Mirtazapine Combinations

The method comprises co-administration of mirtazapine and at least one other active pharmaceutical ingredient. In some embodiments, the method comprises co-administration of mirtazapine and at least one other active pharmaceutical ingredient that improves airflow. The improvement of airflow can be effected by increasing the upper airway muscle tone of the patient during sleep, stabilizing the patient's respiratory drive, or both. In some embodiments of the invention, a compound that provides the effects of increasing the upper airway muscle tone of the patient during sleep and stabilizing the patient's respiratory drive is zonisamide. In other embodiments of the invention, a compound that provides the effect of stabilizing the patient's respiratory drive is topiramate, zonisamide or a 5HT1A agonist, such as buspirone. In still further embodiments, the invention comprises administering to a patient mirtazapine in combination with a compound that treats one or more sequelae of sleep related breathing disorders, such as amantadine, bupropion, modafinil, r-modafinil, SDZ-NVI-085 and amphetamine.

In some embodiments, mirtazapine is combined with one or more compounds that treat one or more symptoms of sleep related breathing disorders, such as daytime sleepiness. For example, mirtazapine can be combined with modafinil, which is effective for the treatment of drowsiness. In some embodiments, mirtazapine and modafinil can be combined in a single dosage form, while in other embodiments they can be prepared in separate dosage forms, and can even be administered at different times in a 24 hour period. (The compound SDZ-NVI-085 can be used in place of modafinil due to its similar alertness-stimulating properties.) In other embodiments, mirtazapine and topiramate, amantadine or bupropion can be combined in a single dosage form, while in other embodiments they can be prepared in separate dosage forms, and can even be administered at different times in a 24 hour period. In another embodiment, administration of mirtazapine nocte can be combined with daytime administration of amphetamine or other compound suitable for treatment of drowsiness.

2. Other Active Pharmaceutical Ingredients

A. Quinacrine. In some embodiments, the invention provides a method of treating or reducing the symptoms of sleep apnea or other sleep related breathing disorders in an individual comprises administering a composition comprising quinacrine or a quinacrine derivative to the individual. In some embodiments, the invention provides administering quinacrine or a quinacrine derivative as the sole pharmaceutically active ingredient. In other embodiments, the invention includes administering quinacrine or a quinacrine derivative along with serotonergic agent selected from the group consisting of a compound that acts as a 5HT1A sertotonin receptor agonist, a serotonin reuptake inhibitor, and a 5HT2A/2C serotonin receptor antagonist, a compound with both selective serotonin reuptake inhibitor and 5HT1A serotonin receptor partial agonist activity, and a compound with both norepinephrine reuptake inhibitor and 5HT3 serotonin receptor antagonist activity. In specific embodiments, the quinacrine or quinacrine derivative has the following formula:

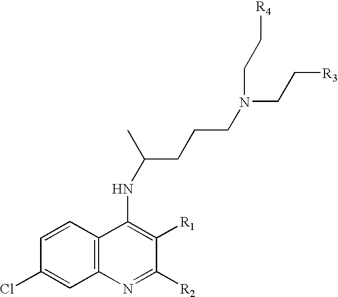

wherein R1 and R2 may be hydrogen, an alkyl group, a hydroxyl group, a methoxy group, a halogen group, an amino group, or together may form a substituted or unsubstituted fused aromatic ring; and wherein R3 and R4 may be hydrogen, a hydroxy group, alkyl group, methoxy group, or halogen group. In certain specific embodiments, the compound is selected from the group consisting of quinacrine, chloroquine, and hydroxychloroquine.

B. Cannabinoid agonists. In some embodiments, the invention provides a method of treating or reducing the symptoms of sleep apnea or other sleep related breathing disorders in an individual, comprising administering to the individual an amount of a cannabinoid agonist sufficient to treat or reduce the symptoms of sleep apnea or other sleep related breathing disorder. In specific embodiments, the cannabinoid agonist is selected from the group consisting of delta-9-THC, delta-8-THC, CP 55940, HU-210, WIN55212-2, O-1057, anandamide, methanandamide, 2-AG, ACEA and ACPA. The cannabinoid agonists can be administered alone, as the sole API, or in combination with another active ingredient. In particular embodiments, a single cannabinoid is administered to the individual as the sole API. In other embodiments, a combination of two or more cannabinoid antagonists may be administered to the individual.

In specific embodiments of the invention, the method of treating or reducing the symptoms of sleep apnea or other sleep related breathing disorders in an individual comprises administering a cannabinoid antagonist and a serotonergic agent selected from the group consisting of a compound that acts as a 5HT1A sertotonin receptor agonist, a serotonin reuptake inhibitor, and a 5HT2A/2C serotonin receptor antagonist, a compound with both selective serotonin reuptake inhibitor and 5HT1A serotonin receptor partial agonist activity, and a compound with both norepinephrine reuptake inhibitor and a 5HT3 serotonin receptor antagonist activity. In specific embodiments, the cannabinoid agonist and the serotonergic agent are co-administered in the same dosage form. In other embodiments, the cannabinoid and the serotonergic agent are administered to the same individual at different times during the day.

C. Acetylcholinesterase Inhibitors. In some embodiments, the invention provides a method of treating or reducing the symptoms of sleep apnea or other sleep related breathing disorders in an individual, comprising administering to the individual an amount of an acetylcholinesterase inhibitor sufficient to treat or reduce the symptoms of sleep apnea or other sleep related breathing disorder. The acetylcholinesterase inhibitor may be administered alone or in combination with another active pharmaceutical ingredient. In some embodiments, the acetylcholinesterase inhibitor is administered alone. In other embodiments, the acetylcholinesterase inhibitor is administered along with at least one other active pharmaceutical ingredient. When administered with another active pharmaceutical ingredient, the acetylcholinesterase inhibitor may be co-administered in the same dose as the other pharmaceutical ingredient or in separate doses at separate times.

In some embodiments, the invention comprises administering to an individual a acetylcholinesterase inhibitor selected from the group consisting of donepezil, galantamine, tacrine, rivastigmine or a combination of two or more thereof.

In particular embodiments in which the acetylcholinesterase inhibitor is administered along with a second compound, the second compound is selected from the group consisting of a compound that acts as a 5HT1A sertotonin receptor agonist, a serotonin reuptake inhibitor, and a 5HT2A/2C serotonin receptor antagonist, a compound having norepinephrine reuptake inhibitor and 5HT3 serotonin receptor antagonist activity, and a compound having selective serotonin reuptake inhibitor and 5HT1A serotonin receptor partial agonist activity.

D. Norepinephrine Inhibitors. In some embodiments, the invention provides a method of treating or reducing the symptoms of sleep apnea or other sleep related breathing disorders in an individual, comprising administering to the individual an amount of a first compound having norepinephrine reuptake inhibitor and 5HT3 serotonin receptor antagonist activity and a second compound having selective serotonin reuptake inhibitor and 5HT1A serotonin receptor partial agonist activity. In such embodiments, the amount of first and second compound administered to the individual is sufficient to treat or reduce the symptoms of sleep apnea or other sleep related breathing disorder. The first and second compounds may be administered in the absence of other active ingredient or in combination with one or more additional active pharmaceutical ingredients.

E. Vilazodone has selective serotonin reuptake inhibitor and 5HT1A serotonin receptor partial antagonist activity. In some embodiments of the invention where the method of treating sleep apnea or another sleep related breathing disorder includes administering a compound having selective serotonin reuptake inhibitor and 5HT1A serotonin receptor partial antagonist activity, the compound having selective serotonin reuptake inhibitor and 5HT1A serotonin receptor partial antagonist activity is vilazodone. Thus, vilazodone may be used alone in combination with quinacrine or a quinacrine derivative as disclosed herein, a cannabinoid agonist as described herein, an acetylcholinesterase inhibitor or a compound having norepinephrine reuptake inhibitor and 5HT3 serotonin receptor antagonist activity. In particular, vilazodone may be used in combination with quinacrine, chloroquine, hydroxychloroquine, Delta-9-THC, Delta-8-THC, CP 55940, HU-210, WIN55212-2, O-1057, anandamide, and methanandamide, 2-AG, ACEA or ACPA. When used in combination therapy, vilazodone may be combined with at least one additional active ingredient in a single dosage form. Alternatively, vilazodone and at least one other active ingredient may be combined in a single dosage form.

F. A combination comprising a first compound that acts as a 5HT1A sertotonin receptor agonist, a serotonin reuptake inhibitor, and a 5HT2A/2C serotonin receptor antagonist and a second compound having norepinephrine reuptake inhibitor and 5HT3 serotonin receptor antagonist activity. In some embodiments, the method of treating or reducing the symptoms of sleep apnea or other sleep related breathing disorders in an individual comprises administering to an individual a first compound that acts as a 5HT1A sertotonin receptor agonist, a serotonin reuptake inhibitor, and a 5HT2A/2C serotonin receptor antagonist and a second compound having norepinephrine reuptake inhibitor and 5HT3 serotonin receptor antagonist activity. In some embodiments, first and second compounds are administered in a single dosage form. In other embodiments, the first and second compounds are administered in separate dosage forms. In some embodiments where the first and second compounds are administered in separate dosage forms, they are administered simultaneously or at substantially different times. In other embodiments, they are administered at substantially different times during the day. invention includes administering quinacrine or a quinacrine derivative along with serotonergic agent selected from the group consisting of a compound that acts as a 5HT1A sertotonin receptor agonist, a serotonin reuptake inhibitor, and a 5HT2A/2C serotonin receptor antagonist, a compound with both selective serotonin reuptake inhibitor and 5HT1A serotonin receptor partial agonist activity, and a compound with both norepinephrine reuptake inhibitor and 5HT3 serotonin receptor antagonist activity.

G. MCI 225 as the compound having Norepinephrine reuptake inhibitor and 5HT3 serotonin receptor antagonist activity. In some embodiments of the invention where the method of treating sleep apnea or another sleep related breathing disorder includes administering a compound having norepinephrine reuptake inhibitor and 5HT3 serotonin receptor antagonist activity, the compound having Norepinephrine reuptake inhibitor and 5HT3 serotonin receptor antagonist activity is MCI 225. Thus, MCI 225 may be used alone in combination with quinacrine or a quinacrine derivative as disclosed herein, a cannabinoid agonist as described herein, an acetylcholinesterase inhibitor or a compound having norepinephrine reuptake inhibitor and 5HT3 serotonin receptor antagonist activity or a combination of a first compound that acts as a 5HT1A sertotonin receptor agonist, a serotonin reuptake inhibitor, and a 5HT2A/2C serotonin receptor antagonist and a second compound having norepinephrine reuptake inhibitor and 5HT3 serotonin receptor antagonist activity. In particular, MCI 225 may be used in combination with quinacrine, chloroquine, hydroxychloroquine, Delta-9-THC, Delta-8-THC, CP 55940, HU-210, WIN55212-2, O-1057, anandamide, and methanandamide, 2-AG, ACEA or ACPA. When used in combination therapy, MCI 225 may be combined with at least one additional active ingredient in a single dosage form. Alternatively, MCI 225 and at least one other active ingredient may be combined in a single dosage form.

H. Methods using a first compound that acts as a 5HT1A serotonin receptor agonist, a serotonin reuptake inhibitor, and a 5HT2A/2C serotonin receptor antagonist and a second compound selected from the group consisting of norepinephrine reuptake inhibitor, norepinephrine/serotonin reuptake inhibitor, alpha-1 agonist, alpha-2 antagonist, quinacrine, a compound that acts as a norepinephrine reuptake inhibitor and a 5HT3 serotonin receptor antagonist, a selective serotonin reuptake inhibitor/5HT1A serotonin receptor partial agonist, a serotonin reuptake inhibitor/5HT3 serotonin receptor antagonist, and a selective serotonin reuptake inhibitor. In some embodiments, the present invention thus provides a method of treating or reducing the symptoms of sleep apnea or other sleep related breathing disorders in an individual, wherein the method comprises administering to the individual an effective amount of a first compound that acts as a 5HT1A serotonin receptor agonist, a serotonin reuptake inhibitor, and a 5HT2A/2C serotonin receptor antagonist and a second compound selected from the group consisting of norepinephrine reuptake inhibitor, norepinephrine/serotonin reuptake inhibitor, alpha-1 agonist, alpha-2 antagonist, quinacrine, a compound that acts as a norepinephrine reuptake inhibitor and a 5HT3 serotonin receptor antagonist, a selective serotonin reuptake inhibitor/5HT1A serotonin receptor partial agonist, a serotonin reuptake inhibitor/5HT3 serotonin receptor antagonist, and a selective serotonin reuptake inhibitor. The first and second compounds may be combined in a single dosage form or may be administered in separate dosage forms, whether simultaneously or at separate times during the day. The first and second compounds are administered in doses sufficient to suppresses REM sleep, increases deep slow wave sleep, and increases the tone of upper airway muscles during sleep and/or increase respiratory drive. In some embodiments, the second compound is selected from the group consisting of SDZ-NVI-085, fipamezole, dexefaroxan, milnacipran, reboxetine, bicifadine, tomoxetine, venlafaxine, desipramine, duloxetine, MC1225, vilazodone, litoxetine, sertraline and citalopram.

I. Trazodone and Derivatives Thereof. In some embodiments of the invention, the method of treating sleep apnea or another sleep related breathing disorder includes administering to an individual a compound having activity as a 5HT1A sertotonin receptor agonist, a serotonin reuptake inhibitor, and a 5HT2A/2C serotonin receptor antagonist, which is trazodone. Thus, trazodone or a derivative thereof may be used alone in combination with quinacrine or a quinacrine derivative as disclosed herein, a cannabinoid agonist as described herein, an acetylcholinesterase inhibitor or a compound having norepinephrine reuptake inhibitor and 5HT3 serotonin receptor antagonist activity. In particular, trazodone or a derivative thereof may be used in combination with quinacrine, chloroquine, hydroxychloroquine, Delta-9-THC, Delta-8-THC, CP 55940, HU-210, WIN55212-2, O-1057, anandamide, and methanandamide, 2-AG, ACEA, ACPA, SDZ-NVI-085, fipamezole, dexefaroxan, milnacipran, reboxetine, bicifadine, tomoxetine, venlafaxine, desipramine, duloxetine, MC1225, vilazodone, litoxetine, sertraline or citalopram. When used in combination therapy, trazodone or a derivative thereof may be combined with at least one additional active ingredient in a single dosage form. Alternatively, trazodone or a derivative thereof and at least one other active ingredient may be combined in a single dosage form. In this regard, the terms “a trazodone,” “trazodones” and “trazodone or a derivative thereof” refer to one or more compounds of the formula:

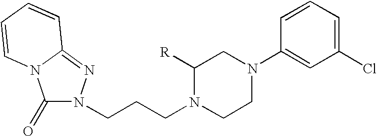

wherein R is hydrogen or an alkyl group having from 1 to 3 carbons, as well as salts and mixtures thereof. In particular embodiments, R is methyl.

Thus, the term “trazodone” by itself refers to a compound of the formula above wherein R is hydrogen.

J. Alpha-2 Antagonist plus SSRI. In some embodiments, the invention includes methods of treating or reducing the symptoms of sleep apnea or other sleep related disorders in an individual, comprising administering to the individual a first compound having alpha-2 antagonist activity in combination with a second compound which is a selective serotonin reuptake inhibitor. In some embodiments, the first compound is selected from the group consisting of fipamezole and dexefaroxan. In some embodiments, the second compound is selected from the group consisting of sertraline and citalopram. In particular embodiments, the method comprises administering, as the first compound, fipamezole, dexefaroxan or a combination thereof, and as the second compounds sertraline, citalopram or a combination thereof. In some embodiments, the first and second compounds are combined in a single dosage; whereas in other embodiments they can be administered in separate dosage forms at the same time or at different times of the day.

In some embodiments, the invention includes methods of treating or reducing the symptoms of sleep apnea or other sleep related disorders in an individual, comprising administering to the individual a first compound having alpha-2 antagonist activity in combination with a second compound which is a 5-HT1A antagonist.

K. 5HT1A Serotonin Receptor Agonist. In some embodiments, the invention includes methods of treating or reducing the symptoms of sleep apnea or other sleep related disorders in an individual, comprising administering to the individual a 5-HT1A serotonin receptor agonist. In some embodiments, the first compound having 5-HT1A agonist activity is administered in combination with a second compound selected from the group consisting of norepinephrine reuptake inhibitors, norepinephrine/serotonin reuptake inhibitors, and alpha-1 adrenergic receptor agonists.

L. 5HT2 Agonists. In some embodiments, the invention includes methods of treating or reducing the symptoms of sleep apnea or other sleep related disorders in an individual, comprising administering to the individual a first compound, which is a 5-HT2A, 5-HT2C or 5-HT2A/2C agonist. In some embodiments, the first compound is selected from the group consisting of milnacipran, reboxetine, tomoxetine, and bicifadine venlafaxine, desipramine, and duloxetine. In some embodiments, the first compound having 5-HT2A, 5-HT2C, or 5-HT2A/2C agonist activity is administered in combination with a second compound selected from the group consisting of norepinephrine reuptake inhibitors, norepinephrine/serotonin reuptake inhibitors, alpha-2 antagonists, and alpha-1 agonists. In other embodiments, the second compound is selected from the group of nonspecific serotonin receptor agonists, such as m-chlorophenylpiperazine, and alpha-1 agonists, such as SDZ NVI-085 {( )-(4aR,10aR)-3,4,4a,5,10,10a-hexahydro-6-methoxy-4-methyl-9-methylthio-2H-naphth 2,3,b-1,4-oxazine HCl}(SDZ-NVI-085).

M. 5-HT2B/2C Antagonists. In some embodiments, the invention includes methods of treating or reducing the symptoms of sleep apnea or other sleep related disorders in an individual, comprising administering to the individual a first compound having melatonin agonistic activity a second compound having 5-HT2B/2C antagonistic activity. In some embodiments, the first compound having melatonin agonistic activity is agomelatine.

N. Serotonin Reuptake Inhibitor/5HT3 Serotonin Receptor Antagonist. In some embodiments, the invention includes methods of treating or reducing the symptoms of sleep apnea or other sleep related disorders in an individual, comprising administering to the individual a serotonin reuptake inhibitor/5HT3 serotonin receptor antagonist. In particular embodiments, the selective serotonin reuptake inhibitor/5HT3 serotonin receptor antagonist is litoxetine. In some embodiments, the method further comprises administering a second compound selected from the group consisting of alpha-1 agonists, alpha-2 antagonists, norepinephrine reuptake inhibitors, norepinephrine/serotonin reuptake inhibitors, monoamine oxidase-B inhibitors, acetylcholinesterase inhibitors, and cannabinoid agonists. In particular embodiments, the second compound is selected from the group consisting of reboxetine, tomoxetine, bicifadine, milnacipran, venlafaxine, desipramine, duloxetine, sertraline and citalopram, fipamezole, dexefaroxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

O. Serotonin Reuptake Inhibitor/5HT2A Serotonin Receptor Antagonist. In some embodiments, the invention includes methods of treating or reducing the symptoms of sleep apnea or other sleep related disorders in an individual, comprising administering to the individual a serotonin reuptake inhibitor/5HT2A serotonin receptor antagonist. In some embodiments, the serotonin reuptake inhibitor/5HT2A serotonin receptor antagonist is selected from the group consisting of LY628535, lubazodone, and R-fluoxetine. In some embodiments, the method comprises administering to the individual a first compound comprising a serotonin reuptake inhibitor/5HT2A serotonin receptor antagonist and a second compound selected from the group consisting of alpha-1 agonists, alpha-2 antagonists, norepinephrine reuptake inhibitors, norepinephrine/serotonin reuptake inhibitors, monoamine oxidase-B inhibitors, acetylcholinesterase inhibitors, cannabinoid agonists, and 5HT3 serotonin receptor antagonists. In particular embodiments, the second compound is selected from the group consisting of reboxetine, tomoxetine, bicifadine, milnacipran, venlafaxine, desipramine, duloxetine, sertraline and citalopram, fipamezole, dexefaroxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

P. Acetylcholine Releaser/Norepinephrine Reuptake Inhibitor Agent. In some embodiments, the invention includes methods of treating or reducing the symptoms of sleep apnea or other sleep related disorders in an individual, comprising administering to the individual an acetylcholine releaser/norepinephrine reuptake inhibitor agent. In some embodiments, the acetylcholine releaser/norepinephrine reuptake inhibitor agent is selected from the group consisting of bifemelane and teniloxazine. In other embodiments, the method comprises administering to the individual a first compound that is a acetylcholine releaser/norepinephrine reuptake inhibitor agent and a second compound selected from the group consisting of alpha-1 agonists, alpha-2 antagonists, selective serotonin reuptake inhibitors, monoamine oxidase-B inhibitors, cannabinoid agonists, 5HT3 serotonin receptor antagonists, 5HT2A serotonin receptor antagonists, serotonin reuptake inhibitor/5HT3 serotonin receptor antagonists, and serotonin reuptake inhibitor/5HT2A serotonin receptor antagonists. In particular embodiments, the second compound is selected from the group consisting of reboxetine, tomoxetine, bicifadine, milnacipran, venlafaxine, desipramine, duloxetine, sertraline and citalopram, fipamezole, dexefaroxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

Q. Co-Administration of a 5HT3 Antagonist and a Second Compound. In some embodiments, the invention includes methods of treating or reducing the symptoms of sleep apnea or other sleep related disorders in an individual, comprising administering to the individual a first compound having 5HT3 antagonist activity and a second compound selected from the group consisting of selective serotonin reuptake inhibitors, alpha-1 agonists, alpha-2 antagonists, norepinephrine reuptake inhibitors, norepinephrine/serotonin reuptake inhibitors, monoamine oxidase-B inhibitors, acetylcholinesterase inhibitors, and cannabinoid agonists. In particular embodiments, the compound having 5HT3 antagonist activity is selected from the group consisting of ondansetron, dolasetron, granisetron, tropisetron, ramosetron, and palonosetron. In particular embodiments, the second compound is selected from the group consisting of reboxetine, tomoxetine, bicifadine, milnacipran, venlafaxine, desipramine, duloxetine, sertraline and citalopram, fipamezole, dexefaroxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

R. 5HT2A Antagonist and a Second Compound. In some embodiments, the invention includes methods of treating or reducing the symptoms of sleep apnea or other sleep related disorders in an individual, comprising administering to the individual a first compound having 5HT2A antagonist activity and a second compound selected from the group consisting of 5HT3 antagonists, selective serotonin reuptake inhibitors, alpha-1 agonists, alpha-2 antagonists, norepinephrine reuptake inhibitors, norepinephrine/serotonin reuptake inhibitors, monoamine oxidase-B inhibitors, acetylcholinesterase inhibitors, and cannabinoid agonists. In some embodiments, the compound having 5HT2A antagonist activity is selected from the group consisting of MDL 100907, EMR-62218, and eplivanserin. In particular embodiments, the second compound is selected from the group consisting of reboxetine, tomoxetine, bicifadine, milnacipran, venlafaxine, desipramine, duloxetine, sertraline and citalopram, fipamezole, dexefaroxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

S. Acetylcholinesterase Inhibitor/Serotonin Reuptake Inhibitor. In some embodiments, the invention includes methods of treating or reducing the symptoms of sleep apnea or other sleep related disorders in an individual, comprising administering to the individual an acetylcholinesterase inhibitor/serotonin reuptake inhibitor agent. In particular embodiments, the acetylcholinesterase inhibitor/serotonin reuptake inhibitor agent is RS-1259. In other embodiments, the method comprises administering, as a first compound, an acetylcholinesterase inhibitor/serotonin reuptake inhibitor agent, and as a second compound, a member selected from the group consisting of norepinephrine reuptake inhibitors, norepinephrine/serotonin reuptake inhibitors, alpha-1 agonists, alpha-2 antagonists, monoamine oxidase-B inhibitors, 5HT2A serotonin receptor antagonists, 5HT3 serotonin receptor antagonists, or cannabinoid agonists. In particular embodiments, the second compound is selected from the group consisting of reboxetine, tomoxetine, bicifadine, milnacipran, venlafaxine, desipramine, duloxetine, sertraline and citalopram, fipamezole, dexefaroxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

T. 5HT2/5HT3 Serotonin Receptor Antagonist and Alpha-2 Adrenergic Receptor Antagonist. In some embodiments, the invention includes methods of treating or reducing the symptoms of sleep apnea or other sleep related disorders in an individual, comprising administering to the individual a compound having 5HT2/5HT3 serotonin receptor antagonist and alpha-2 adrenergic receptor antagonist activity. In particular embodiment, the compound having 5HT2/5HT3 serotonin receptor antagonist and alpha-2 adrenergic receptor antagonist activity is mirtazapine. In especially notable embodiments, the method comprises administering a first compound having 5HT2/5HT3 serotonin receptor antagonist and alpha-2 adrenergic receptor antagonist activity (such as mirtazapine) and a second compound selected from the group consisting of acetylcholinesterase inhibitors, selective serotonin reuptake inhibitors, norepinephrine reuptake inhibitors, norepinephrine/serotonin reuptake inhibitors, alpha-1 adrenergic receptor agonists, acetylcholinesterase inhibitors/serotonin reuptake inhibitor agents, acetylcholine releaser/norepinephrine reuptake inhibitor agents, monoamine oxidase-B inhibitors, and cannabinoid agonists.

U. Ergot Alkaloid. In some embodiments, the invention includes methods of treating or reducing the symptoms of sleep apnea or other sleep related disorders in an individual, comprising administering to the individual an ergot alkaloid with serotonin receptor agonist activity. In particular embodiments, the serotonin receptor agonist agonizes the 5HT1A, 5HT1D or 5HT2A receptor.

In some embodiments, the ergot alkaloid is selected from the group consisting of ergotamine, dihydroergotamine, acetergamine, brazergoline, bromerguride, cianergoline, delorgotrile, disulergine, ergonovine maleate, etisulergine, lergotrile, lysergide, mesulergine, metergoline, metergotamine, nicergoline, pergolide, propisergide, proterguride, terguride and combinations thereof.

V. Treatment of Sleep Apnea. In some embodiments, the invention provides a method of any one of sections A-U above, wherein the compound is, or compounds are, administered in an amount effective to treat or alleviate the symptoms of sleep apnea.

W. Specific Sleep related Breathing Disorders. In some embodiments, the invention provides a method of treating or reducing the symptoms of a condition associated with sleep related breathing disorders in an individual, comprising administering an effective amount of an alpha-1 adrenergic receptor agonist. In particular embodiments, the condition associated with sleep related breathing disorders is excessive daytime sleepiness.

In some embodiments, the method of treating or reducing the symptoms of a condition associated with sleep related breathing disorders in an individual further comprises administering a compound that improves the hypoxic index selected from the group consisting of a 5HT1A sertotonin receptor agonist/serotonin reuptake inhibitor/5HT2A/2C serotonin receptor antagonist, selective serotonin reuptake inhibitor/5HT1A serotonin receptor partial agonists, norepinephrine reuptake inhibitor/5HT3 serotonin receptor antagonists, melatonin agonists, selective reuptake inhibitor/5HT3 serotonin receptor antagonists, selective reuptake inhibitor/5HT2A receptor antagonists, acetylcholine releaser/norepinephrine reuptake agents, 5HT2A serotonin receptor antagonists, and acetylcholinesterase inhibitor/serotonin reuptake inhibitor agents, and 5HT2/5HT3 antagonist/alpha-2 antagonists. In particular embodiments, the alpha-1 adrenergic receptor agonist is selected from the group consisting of SDZ-NVI-085, modafinil and adrafinil. In particular embodiments, the compound that improves the hypoxic index is selected from the group consisting of MER 810, vilazodone, MCI 225, agomelatine, litoxetine, LY628535, lubazodone, R-fluoxetine, bifemelane, teniloxazine, MDL100907, eplivanserin, RS-1259, mirtazapine.

In other embodiments, the invention provides a method of reducing the side effects of a compound administered to improve the hypoxic index in an individual to treat or reduce the symptoms of sleep apnea or other sleep related breathing disorders selected from the group consisting of 5HT1A sertotonin receptor agonist/serotonin reuptake inhibitor/5HT2A/2C serotonin receptor antagonist, selective serotonin reuptake inhibitor/5HT1A serotonin receptor partial agonists, norepinephrine reuptake inhibitor/5HT3 serotonin receptor antagonists, melatonin agonists, selective reuptake inhibitor/5HT3 serotonin receptor antagonists, selective reuptake inhibitor/5HT2A receptor antagonists, acetylcholine releaser/norepinephrine reuptake agents, 5HT2A serotonin receptor antagonists, and acetylcholinesterase inhibitor/serotonin reuptake inhibitor agents, and 5HT2/5HT3 antagonist/alpha-2 antagonists comprising administering an effective amount of an alpha-1 adrenergic receptor agonist. In particular embodiments, the alpha-1 adrenergic receptor agonist is selected from the group consisting of SDZ-NVI-085, modafinil and adrafinil. In particular embodiments, the compound that improves the hypoxic index is selected from the group consisting of MER 810, vilazodone, MCI 225, agomelatine, litoxetine, LY628535, lubazodone, R-fluoxetine, bifemelane, teniloxazine, MDL100907, eplivanserin, RS-1259, and mirtazapine.

In other embodiments, the invention provides a method for reducing one or more symptoms in an individual, which are associated with the use of a 5HT2/5HT3 antagonist/alpha-2 antagonist in the treatment of sleep related breathing disorders, selected from the group consisting of excessive daytime drowsiness and weight gain comprising administering an effective amount of a compound selected from the group consisting of quinacrine, norepinephrine reuptake inhibitor/dopamine reuptake inhibitor compound, norepinephrine reuptake inhibitor, norepinephrine/serotonin reuptake inhibitor, D1 dopamine receptor agonist, 5HT1A serotonin receptor agonist, carbonic anhydrase inhibitor, and dopamine-releasing compound. In some such embodiments, the 5HT2/5HT3 antagonist/alpha-2 antagonist is mirtazapine. In other such embodiments, the norepinephrine reuptake inhibitor/dopamine reuptake inhibitor compound is bupropion. In still other embodiments, the norepinephrine reuptake inhibitor is selected from the group consisting of atomoxetine, reboxetine, tomoxetine, and bicifadine and the norepinephrine/serotonin reuptake inhibitor is selected from the group consisting of milnacipran, venlafaxine, desipramine and duloxetine. And in still further embodiments, the D1 dopamine receptor agonist is selected from the group consisting of SKF38393, CY-208-243, dihydrexidine, SKF82958, A77636 and A68930. While in other embodiments, 2 wherein the 5HT1A serotonin receptor agonist is selected from the group consisting of buspirone, gepirone, and alnespirone and Org 13011. In other embodiments, the carbonic anhydrase inhibitor is selected from the group consisting of acetazolamide, zonisamide, methazolamide, dichlorphenamide, and topiramate. In still further embodiments, the dopamine-releasing compound is selected from the group consisting of amantadine, rimantadine, methamphetamine, dextroamphetamine, laevoamphetamine and methylphenidate.

X. Improving patient response to positive air pressure (PAP) treatment. In some embodiments, the invention provides a method for reducing the pressure applied during positive air pressure (PAP) therapy, such as continuous positive airway pressure (CPAP) therapy, in the treatment of sleep related breathing disorders in an individual. The method comprises administering an effective amount of one or more compounds that suppress REM sleep, increases deep slow wave sleep, or increases the tone of upper airway muscles during sleep and/or increase respiratory drive.

In some embodiments, the invention provides a method for reducing the pressure applied during continuous positive airway pressure therapy in the treatment of sleep related breathing disorders in an individual comprising administration of an effective amount of one or more compounds selected from the group consisting of quinacrine or a derivative thereof, acetylcholinesterase inhibitors, cannabinoid agonists, 5HT1A sertotonin receptor agonist/serotonin reuptake inhibitor/5HT2A/2C serotonin receptor antagonists, alpha-2 adrenergic receptor antagonists, 5-HT1A serotonin receptor agonists, 5HT2A/2C serotonin receptor agonists, melatonin agonists, 5HT3 serotonin receptor antagonists, 5HT2A serotonin receptor antagonists, selective serotonin reuptake inhibitors, norepinephrine reuptake inhibitors, norepinephrine/serotonin reuptake inhibitors, alpha-1 adrenergic receptor agonists, monoamine oxidase (MAO)-B inhibitors, ergot alkaloids, 5HT2B/2C serotonin receptor antagonists, serotonin reuptake inhibitor/5HT3 serotonin receptor antagonists, serotonin reuptake inhibitor/5HT2A serotonin receptor antagonists, acetylcholine releaser/norepinephrine reuptake inhibitor agents, norepinephrine reuptake inhibitor/5-HT3 serotonin receptor antagonists, selective serotonin reuptake inhibitor/5HT1A serotonin receptor partial agonists, acetylcholinesterase inhibitor/serotonin reuptake inhibitor agents, 5HT2 antagonist/alpha-1 agonists, 5HT2/5HT3 serotonin receptor antagonist/alpha-2 adrenergic receptor antagonists, and norepinephrine reuptake inhibitor/dopamine reuptake inhibitor compounds.

In some embodiments, the invention provides a method for reducing the pressure applied during continuous positive airway pressure therapy in the treatment of sleep related breathing disorders in an individual comprising administration of an effective amount of a combination of two drugs, wherein the drug combination is selected from the group of combinations consisting of 5HT2/5HT3 antagonist/alpha-2 antagonists and norepinephrine reuptake inhibitor/dopamine reuptake inhibitor compounds, 5HT3 antagonists and 5HT2A antagonists, 5HT3 antagonists and serotonin reuptake inhibitor/5HT2A antagonists, 5HT3 antagonists, 5HT1A sertotonin receptor agonist/serotonin reuptake inhibitor/5HT2A/2C serotonin receptor antagonists, and 5HT3 antagonists and 5HT2 antagonist/alpha-i agonists.

Y. Corticosteroid Plus a Second Compound. In other embodiments, the invention provides a of treating sleep related breathing disorders comprising administering an effective amount of a corticosteroid in combination with a compound selected from the group of compounds consisting of quinacrine, acetylcholinesterase inhibitors, cannabinoid agonists, 5HT1A sertotonin receptor agonist/serotonin reuptake inhibitor/5HT2A/2C serotonin receptor antagonists, alpha-2 adrenergic receptor antagonists, 5-HT1A serotonin receptor agonists, 5HT2A/2C serotonin receptor agonists, melatonin agonists, 5HT3 serotonin receptor antagonists, 5HT2A serotonin receptor antagonists, selective serotonin reuptake inhibitors, norepinephrine reuptake inhibitors, norepinephrine/serotonin reuptake inhibitors, alpha-1 adrenergic receptor agonists, monoamine oxidase (MAO)-B inhibitors, ergot alkaloids, dopamine D1 receptor agonists, carbonic anhydrase inhibitors, 5HT2B/2C serotonin receptor antagonists, serotonin reuptake inhibitor/5HT3 serotonin receptor antagonists, serotonin reuptake inhibitor/5HT2A serotonin receptor antagonists, acetylcholine releaser/norepinephrine reuptake inhibitor agents, norepinephrine reuptake inhibitor/5-HT3 serotonin receptor antagonists, selective serotonin reuptake inhibitor/5HT1A serotonin receptor partial agonists, acetylcholinesterase inhibitor/serotonin reuptake inhibitor agents, 5HT2 antagonist/alpha-1 agonists, 5HT2/5HT3 serotonin receptor antagonist/alpha-2 adrenergic receptor antagonists, and norepinephrine reuptake inhibitor/dopamine reuptake inhibitor. In particular embodiments, the corticosteroid is selected from the group consisting of hydrocortisone, cortisone, dexamethasone, and prednisone.

Z. Carbonic Anhydrase Inhibitors. In some embodiments, the invention includes methods of treating or reducing the symptoms of sleep apnea or other sleep related disorders in an individual, comprising administering to the individual an effective amount of a carbonic anhydrase inhibitor. In some embodiments, the method comprises administering a second compound selected from the group consisting of 5HT1A sertotonin receptor agonist/serotonin reuptake inhibitor/5HT2A/2C serotonin receptor antagonists, selective serotonin reuptake inhibitors, norepinephrine/serotonin reuptake inhibitors, norepinephrine reuptake inhibitors, dopamine/serotonin receptor antagonists, norepinephrine reuptake inhibitor/5HT3 serotonin receptor antagonists, serotonin reuptake inhibitor/5HT3 serotonin reuptake inhibitor antagonists, and selective reuptake inhibitor/5HT2A serotonin receptor antagonists.

In some embodiments, the carbonic anhydrase inhibitor is acetazolamide, zonisamide, methazolamide, dichlorphenamide, and topiramate. In this regard, zonisamide and topiramate are considered particularly advantageous. In particular, zonisamide provides the combined effects of improving upper airway muscle tone during sleep and stabilizing respiratory drive.

Formulations for Treating Sleep Apnea or other Sleep Related Breathing Disorders. The invention also provides particular formulations for the treatment of sleep apnea and/or other sleep related disorders. In particular, the invention provides formulations for treating or reducing the symptoms of sleep apnea or other sleep related breathing disorders in an individual comprising an effective amount of one or more compounds to suppress REM sleep, increase deep slow wave sleep, and increase the tone of upper airway muscles during sleep and/or increase respiratory drive.

AA. Quinacrine Formulations. In some embodiments, the invention provides a formulation comprising quinacrine or a derivative of quinacrine. In particular embodiments, the invention provides quinacrine or a derivative of quinacrine in a unit dosage form. In particular embodiments, the formulation comprises quinacrine or a derivative of quinacrine in a unit dosage form suitable for providing an amount of quinacrine or a quinacrine derivative sufficient to treat or reduce the symptoms of sleep apnea or other sleep related breathing disorders in an individual. In some embodiments, the formulation comprises quinacrine or a derivative of quinacrine in combination with at least a second compound in a unit dosage form. In particular embodiments, the combination of quinacrine or quinacrine derivative and second compound are present in the unit dosage form in an amount suitable for providing an amount of the combination sufficient to treat or reduce the symptoms of sleep apnea or other sleep related breathing disorders in an individual. In particular embodiments, the second compound is a serotonergic agent selected from the group consisting of a 5HT1A sertotonin receptor agonist/serotonin reuptake inhibitor/5HT2A/2C serotonin receptor antagonist, a compound with both selective serotonin reuptake inhibitor and 5HT1A partial agonist activity, and a compound with both norepinephrine reuptake inhibitor and a 5HT3 serotonin receptor antagonist activity.

In some particular embodiments of the invention, the compound having selective serotonin reuptake inhibitor and 5HT1A partial agonist activity is vilazodone.

In some particular embodiments, wherein the formulation comprises a compound having norepinephrine reuptake inhibitor and 5HT3 serotonin receptor antagonist activity, such compound is MCI 225.

In particular embodiments, the quinacrine or quinacrine derivative is compounds having the following formula:

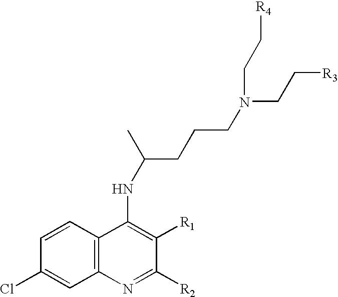

wherein R1 and R2 may be hydrogen, an alkyl group, a hydroxyl group, a methoxy group, a halogen group, an amino group, or together may form a substituted or unsubstituted fused aromatic ring; and

wherein R3 and R4 may be hydrogen, a hydroxy group, alkyl group, methoxy group, or halogen group.

In particular embodiments, the quinacrine or quinacrine derivative is selected from the group consisting of quinacrine, chloroquine, and hydroxychloroquine.

BB. Cannabinoid Agonist Formulations. In some embodiments, the invention provides a formulation comprising a cannabinoid agonist. In particular embodiments, the invention provides a cannabinoid agonist in a unit dosage form. In particular embodiments, the formulation comprises a cannabinoid agonist in a unit dosage form suitable for providing an amount of a cannabinoid agonist sufficient to treat or reduce the symptoms of sleep apnea or other sleep related breathing disorders in an individual. In some embodiments, the formulation comprises a cannabinoid agonist in combination with at least a second compound in a unit dosage form. In particular embodiments, the combination of a cannabinoid agonist and at least a second compound are present in the unit dosage form in an amount suitable for providing an amount of the combination sufficient to treat or reduce the symptoms of sleep apnea or other sleep related breathing disorders in an individual. In particular embodiments, the second compound is a serotonergic agent selected from the group consisting of a 5HT1A sertotonin receptor agonist/serotonin reuptake inhibitor/5HT2A/2C serotonin receptor antagonist, a compound with both selective serotonin reuptake inhibitor and 5HT1A partial agonist activity, and a compound with both norepinephrine reuptake inhibitor and a 5HT3 serotonin receptor antagonist activity.

In some particular embodiments of the invention, the compound having selective serotonin reuptake inhibitor and 5HT1A partial agonist activity is vilazodone.

In some particular embodiments, wherein the formulation comprises a compound having norepinephrine reuptake inhibitor and 5HT3 serotonin receptor antagonist activity, such compound is MCI 225.

In particular embodiments, the invention provides a formulation comprising a cannabinoid agonist selected from the group consisting of Delta-9-THC, Delta-8-THC, CP 55940, HU-210, WIN55212-2, O-1057, anandamide, methanandamide, 2-AG, ACEA and ACPA. In more particular embodiments, the invention provides a cannabinoid agonist cannabinoid agonist selected from the group consisting of Delta-9-THC, Delta-8-THC, CP 55940, HU-210, WIN55212-2, O-1057, anandamide, methanandamide, 2-AG, ACEA and ACPA in combination with a serotonergic agent selected from the group consisting of a 5HT1A sertotonin receptor agonist/serotonin reuptake inhibitor/5HT2A/2C serotonin receptor antagonist, a compound with both selective serotonin reuptake inhibitor and 5HT1A partial agonist activity, and a compound with both norepinephrine reuptake inhibitor and 5HT3 serotonin receptor antagonist activity. In more particular embodiments of the invention, the compound having selective serotonin reuptake inhibitor and 5HT1A partial agonist activity is vilazodone.

CC. Acetylcholinesterase Inhibitor Formulations. In some embodiments, the invention provides a formulation comprising an acetylcholinesterase inhibitor. In particular embodiments, the invention provides an acetylcholinesterase inhibitor in a unit dosage form. In particular embodiments, the formulation comprises an acetylcholinesterase inhibitor in a unit dosage form suitable for providing an amount of an acetylcholinesterase inhibitor sufficient to treat or reduce the symptoms of sleep apnea or other sleep related breathing disorders in an individual. In some embodiments, the formulation comprises an acetylcholinesterase inhibitor in combination with at least a second compound in a unit dosage form. In particular embodiments, the combination of an acetylcholinesterase inhibitor and at least a second compound are present in the unit dosage form in an amount suitable for providing an amount of the combination sufficient to treat or reduce the symptoms of sleep apnea or other sleep related breathing disorders in an individual. In particular embodiments, the second compound is a serotonergic agent selected from the group consisting of a 5HT1A sertotonin receptor agonist/serotonin reuptake inhibitor/5HT2A/2C serotonin receptor antagonist, a compound with both selective serotonin reuptake inhibitor and 5HT1A partial agonist activity, and a compound with both norepinephrine reuptake inhibitor and a 5HT3 serotonin receptor antagonist activity.

In some particular embodiments of the invention, the compound having selective serotonin reuptake inhibitor and 5HT1A partial agonist activity is vilazodone.

In some particular embodiments, wherein the formulation comprises a compound having norepinephrine reuptake inhibitor and 5HT3 serotonin receptor antagonist activity, such compound is MCI 225.

In some embodiments, the formulation comprises an acetylcholinesterase inhibitor that is selected from the group consisting of donepezil, galantamine, tacrine, and rivastigmine. In particular embodiments, in addition to an acetylcholinesterase inhibitor selected from donepezil, galantamine, tacrine and rivastigimine, the formulation also contains a second compound selected from the group consisting of a 5HT1A sertotonin receptor agonist/serotonin reuptake inhibitor/5HT2A/2C serotonin receptor antagonist, a compound having norepinephrine reuptake inhibitor and 5HT3 serotonin receptor antagonist activity, and a compound having selective serotonin reuptake inhibitor and 5HT1A partial agonist activity. In more particular embodiments of the invention, the compound having selective serotonin reuptake inhibitor and 5HT1A partial agonist activity is vilazodone.

CC. Formulation of a Compound Having Norepinephrine Reuptake Inhibitor and 5HT3 Serotonin Receptor Antagonist and A Compound Having Selective Serotonin Reuptake Inhibitor and 5HT1A partial Agonist Activity. In some embodiments, the invention provides a formulation comprising first compound having norepinephrine reuptake inhibitor and 5HT3 serotonin receptor antagonist activity and a second compound having selective serotonin reuptake inhibitor and 5HT1A partial agonist activity. In particular embodiments, the invention provides first compound having norepinephrine reuptake inhibitor and 5HT3 serotonin receptor antagonist activity and a second compound having selective serotonin reuptake inhibitor and 5HT1A partial agonist activity in a unit dosage form. In particular embodiments, the formulation comprises an first compound having norepinephrine reuptake inhibitor and 5HT3 serotonin receptor antagonist activity and a second compound having selective serotonin reuptake inhibitor and 5HT1A partial agonist activity in a unit dosage form suitable for providing an amount of the combination of the first and second compounds sufficient to treat or reduce the symptoms of sleep apnea or other sleep related breathing disorders in an individual. In some embodiments, the formulation comprises the first and second compounds in combination with at least a third compound in a unit dosage form. In particular embodiments, the combination of the first, second and at least a third compound are present in the unit dosage form in an amount suitable for providing an amount of the combination sufficient to treat or reduce the symptoms of sleep apnea or other sleep related breathing disorders in an individual. In particular embodiments, the third compound is a serotonergic agent selected from the group consisting of a 5HT1A sertotonin receptor agonist/serotonin reuptake inhibitor/5HT2A/2C serotonin receptor antagonist, a compound with both selective serotonin reuptake inhibitor and 5HT1A partial agonist activity, and a compound with both norepinephrine reuptake inhibitor and a 5HT3 serotonin receptor antagonist activity.

In some particular embodiments of the invention, the compound having selective serotonin reuptake inhibitor and 5HT1A partial agonist activity is vilazodone.

In some particular embodiments, wherein the formulation comprises a compound having norepinephrine reuptake inhibitor and 5HT3 serotonin receptor antagonist activity, such compound is MCI 225.

DD. Formulation 5HT1A Sertotonin Receptor Agonist/Serotonin Reuptake Inhibitor/5HT2A/2C Serotonin Receptor Antagonist and a Second Compound. In some embodiments, the invention provides a formulation comprising first compound that is a 5HT1A sertotonin receptor agonist/serotonin reuptake inhibitor/5HT2A/2C serotonin receptor antagonist and a second compound selected from the group consisting of alpha-1 agonists, alpha-2 antagonists, norepinephrine reuptake inhibitors, norepinephrine/serotonin reuptake inhibitors, compounds that act as a norepinephrine reuptake inhibitor and a 5HT3 serotonin receptor antagonist, a selective serotonin reuptake inhibitor/5HT1A serotonin receptor partial agonist, a serotonin reuptake inhibitor/5HT3 serotonin receptor antagonist, and a selective serotonin reuptake inhibitor. In particular embodiments, the invention provides a formulation comprising the first and second compounds in unit dosage form. In particular embodiments, the formulation comprises the first and second compounds in a unit dosage form suitable for providing an amount of the combination of the first and second compounds sufficient to treat or reduce the symptoms of sleep apnea or other sleep related breathing disorders in an individual. In some embodiments, the formulation comprises the first and second compounds in combination with at least a third compound in a unit dosage form. In particular embodiments, the combination of the first, second and at least a third compound are present in the unit dosage form in an amount suitable for providing an amount of the combination sufficient to treat or reduce the symptoms of sleep apnea or other sleep related breathing disorders in an individual.

In some particular embodiments of the invention; the compound having selective serotonin reuptake inhibitor and 5HT1A partial agonist activity is vilazodone.

In particular embodiments under this section, the formulation comprises a second compound that is selected from the group consisting of fipamezole, dexeferoxan, milnacipran, reboxetine, bicifadine, and tomoxetine, venlafaxine, desipramine, and duloxetine, MCI225, vilazodone, litoxetine, sertraline and citalopram.

EE. Formulations Containing Trazodone and/or Trazodone Derivatives. In particular embodiments under this sections AA-DD above, the formulation contains one or more compound having 5HT1A sertotonin receptor agonist/serotonin reuptake inhibitor/5HT2A/2C serotonin receptor antagonist activity. In specific embodiments, such compound having 5HT1A sertotonin receptor agonist/serotonin reuptake inhibitor/5HT2A/2C serotonin receptor antagonist activity is trazodone or a derivative of trazodone selected from those compounds having the general formula:

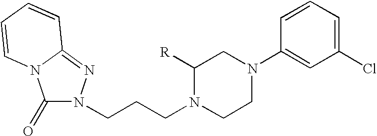

wherein R is hydrogen or an alkyl group having from 1 to 3 carbons.

In particular embodiments, the formulation contains a compound of the above formula, wherein the variable R is methyl.

FF. Formulation of Alpha-2 Antagonist and a SSRI. In some embodiments, the invention provides a formulation comprising a first compound having alpha-2 antagonist activity in combination with a second compound which is a selective serotonin reuptake inhibitor. In particular embodiments, the invention provides a formulation comprising the alpha-2 antagonist and the selective serotonin reuptake inhibitor in unit dosage form. In particular embodiments, the formulation comprises such first and second compounds in a unit dosage form suitable for providing an amount of the combination of the first and second compounds sufficient to treat or reduce the symptoms of sleep apnea or other sleep related breathing disorders in an individual. In some embodiments, the formulation comprises the first and second compounds in combination with at least a third compound in a unit dosage form. In particular embodiments, the combination of the first, second and at least a third compound are present in the unit dosage form in an amount suitable for providing an amount of the combination sufficient to treat or reduce the symptoms of sleep apnea or other sleep related breathing disorders in an individual.

In some particular embodiments of the invention, the first compound is selected from the group consisting of fipamezole and dexeferoxan.

In particular embodiments under this section, the second compound is selected from the group consisting of sertraline and citalopram.

GG. Formulation of an Alpha-2 Antagonist and a 5-HT1A Antagonist. In some embodiments, the invention provides a formulation comprising a first compound having alpha-2 antagonist activity in combination with a second compound which is a 5-HT1A antagonist. In particular embodiments, the invention provides a formulation comprising the alpha-2 antagonist and the 5-HT1A antagonist in unit dosage form. In particular embodiments, the formulation comprises such first and second compounds in a unit dosage form suitable for providing an amount of the combination of the first and second compounds sufficient to treat or reduce the symptoms of sleep apnea or other sleep related breathing disorders in an individual. In some embodiments, the formulation comprises the first and second compounds in combination with at least a third compound in a unit dosage form. In particular embodiments, the combination of the first, second and at least a third compound are present in the unit dosage form in an amount suitable for providing an amount of the combination sufficient to treat or reduce the symptoms of sleep apnea or other sleep related breathing disorders in an individual.

HH. Formulation of a 5-HT1A Agonist. In some embodiments, the invention provides a formulation comprising a 5-HT1A agonist. In particular embodiments, the invention provides a formulation comprising the 5-HT1A agonist in unit dosage form. In particular embodiments, the formulation comprises such 5-HT1A agonist in a unit dosage form suitable for providing an amount of the combination of the 5-HT1A agonist sufficient to treat or reduce the symptoms of sleep apnea or other sleep related breathing disorders in an individual. In some embodiments, the formulation comprises the 5-HT1A agonist in combination with a second compound in a unit dosage form. In particular embodiments, the combination of the first and second compounds are present in the unit dosage form in an amount suitable for providing an amount of the combination sufficient to treat or reduce the symptoms of sleep apnea or other sleep related breathing disorders in an individual.

In some embodiments, the invention provides a formulation that is a combination of a first compound having 5-HT1A agonist activity and a second compound selected from the group consisting of norepinephrine reuptake inhibitors, norepinephrine/serotonin reuptake inhibitors, and alpha-1 agonists. In particular embodiments, the formulation comprises a second compound selected from the group consisting of milnacipran, reboxetine, tomoxetine, bicifadine venlafaxine, desipramine, and duloxetine.

II. Formulation of a 5-HT2A, 5-HT2C or 5-HT2A/2C Agonist. In some embodiments, the invention provides a formulation comprising a 5-HT2A, 5-HT2C or 5-HT2A/2C agonist. In particular embodiments, the invention provides a formulation comprising the 5-HT2A, 5-HT2C or 5-HT2A/2C agonist in unit dosage form. In particular embodiments, the formulation comprises such 5-HT2A, 5-HT2C or 5-HT2A/2C agonist in a unit dosage form suitable for providing an amount of the combination of the 5-HT2A, 5-HT2C or 5-HT2A/2C agonist sufficient to treat or reduce the symptoms of sleep apnea or other sleep related breathing disorders in an individual. In some embodiments, the formulation comprises the 5-HT2A, 5-HT2C or 5-HT2A/2C agonist in combination with a second compound in a unit dosage form. In particular embodiments, the combination of the first and second compounds are present in the unit dosage form in an amount suitable for providing an amount of the combination sufficient to treat or reduce the symptoms of sleep apnea or other sleep related breathing disorders in an individual.

In some embodiments, the invention provides a formulation comprising a first compound having 5-HT2A, 5-HT2C, or 5-HT2A/2C agonist activity in combination with a second compound selected from the group consisting of norepinephrine reuptake inhibitors, norepinephrine/serotonin reuptake inhibitors, alpha-2 antagonists, and alpha-1 agonists. In particular embodiments of the invention, the formulation further comprises the nonspecific serotonin receptor agonist, m-chlorophenylpiperazine, and the norepinephrine alpha1 agonist, SDZ NVI-085 [( )-(4aR,10aR)-3,4,4a,5,10,10a-hexahydro-6-methoxy-4-methyl-9-methylthio-2H-naphth 2,3,b-1,4-oxazine HCl].

JJ. Formulation of a Combination of a Melatonin Agonist and a 5-HT2B/2C Antagonist. In some embodiments, the invention provides a formulation comprising a first compound having melatonin agonist activity in combination with a second compound having 5-HT2B/2C antagonistic activity. In particular embodiments, the invention provides a formulation comprising a melatonin agonist in combination with a 5-HT2B/2C antagonist in unit dosage form. In particular embodiments, the formulation comprises such combination of a melatonin agonist and a 5-HT2B/2C antagonist in a unit dosage form suitable for providing an amount of the combination sufficient to treat or reduce the symptoms of sleep apnea or other sleep related breathing disorders in an individual. In some embodiments, the formulation comprises the melatonin agonist in combination with the 5-HT2B/2C antagonist, further in combination with a third compound in a unit dosage form. In particular embodiments, the combination of the first, second and third compounds are present in the unit dosage form in an amount suitable for providing an amount of the combination sufficient to treat or reduce the symptoms of sleep apnea or other sleep related breathing disorders in an individual.

In particular embodiments, the invention provides a formulation comprising a melatonin agonist in combination with a 5-HT2B/2C antagonist, wherein the melatonin agonist is agomelatine.

KK. Formulation Containing a Serotonin Reuptake Inhibitor/5HT3 Serotonin Receptor Antagonist. In some embodiments, the invention provides a formulation comprising a compound having serotonin reuptake inhibitor/5HT3 serotonin receptor antagonist activity. In particular embodiments, the invention provides a formulation comprising a serotonin reuptake inhibitor/5HT3 serotonin receptor antagonist in unit dosage form. In particular embodiments, the formulation comprises such serotonin reuptake inhibitor/5HT3 serotonin receptor antagonist in a unit dosage form suitable for providing an amount of the combination sufficient to treat or reduce the symptoms of sleep apnea or other sleep related breathing disorders in an individual. In some embodiments, the formulation comprises the serotonin reuptake inhibitor/5HT3 serotonin receptor antagonist in combination with a second compound in a unit dosage form. In particular embodiments, the combination of the first and second compounds are present in the unit dosage form in an amount suitable for providing an amount of the combination sufficient to treat or reduce the symptoms of sleep apnea or other sleep related breathing disorders in an individual.

In particular embodiments, the invention provides a formulation containing the serotonin reuptake inhibitor/5HT3 serotonin receptor antagonist litoxetine.

In some embodiments, the invention provides a formulation containing a serotonin reuptake inhibitor/5HT3 serotonin receptor antagonist and a second compound, wherein the second compound is selected from the group consisting of alpha-1 agonists, alpha-2 antagonists, norepinephrine reuptake inhibitors, norepinephrine/serotonin reuptake inhibitors, monoamine oxidase-B inhibitors, acetylcholinesterase inhibitors, and cannabinoid agonists. In particular embodiments, such a formulation comprises a member of the group consisting of reboxetine, tomoxetine, bicifadine, milnacipran, venlafaxine, desipramine, duloxetine, sertraline and citalopram, fipamezole, dexeferoxan, SDZ-NVI-085, donepezil, galantamine, tetrahydroaminoacridine, rivastigmine, Delta-9-THC, Delta-8-THC, CP 55940, HU-2 10, WIN552 12-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

LL. Formulation Containing a Serotonin Reuptake Inhibitor/5HT2A Serotonin Receptor Antagonist. In some embodiments, the invention provides a formulation comprising a serotonin reuptake inhibitor/5HT2A serotonin receptor antagonist. In particular embodiments, the invention provides a formulation comprising a serotonin reuptake inhibitor/5HT2A serotonin receptor antagonist in unit dosage form. In particular embodiments, the formulation comprises such serotonin reuptake inhibitor/5HT2A serotonin receptor antagonist in a unit dosage form suitable for providing an amount of the combination sufficient to treat or reduce the symptoms of sleep apnea or other sleep related breathing disorders in an individual. In some embodiments, the formulation comprises the serotonin reuptake inhibitor/5HT2A serotonin receptor antagonist in combination with a second compound in a unit dosage form. In particular embodiments, the combination of the first and second compounds are present in the unit dosage form in an amount suitable for providing an amount of the combination sufficient to treat or reduce the symptoms of sleep apnea or other sleep related breathing disorders in an individual. In particular embodiments, the serotonin reuptake inhibitor/5HT2A serotonin receptor antagonist is selected from the group consisting of LY628535, lubazodone, and R-fluoxetine.

In some embodiments, the invention provides a formulation comprising a first compound having serotonin reuptake inhibitor/5HT2A serotonin receptor antagonist activity and a second compound selected from the group consisting of alpha-1 agonists, alpha-2 antagonists, norepinephrine reuptake inhibitors, norepinephrine/serotonin reuptake inhibitors, monoamine oxidase-B inhibitors, acetylcholinesterase inhibitors, cannabinoid agonists, and 5HT3 antagonists. In some such embodiments, the invention provides a formulation comprising a first compound having serotonin reuptake inhibitor/5HT2A serotonin receptor antagonist activity and a second compound selected from the group consisting of reboxetine, tomoxetine, bicifadine, milnacipran, venlafaxine, desipramine, duloxetine, sertraline and citalopram, fipamezole, dexeferoxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

MM. Formulation Containing an Acetylcholine Releaser/Norepinephrine Reuptake Inhibitor Agent. In some embodiments, the invention provides a formulation comprising an acetylcholine releaser/norepinephrine reuptake inhibitor agent. In particular embodiments, the invention provides a formulation comprising an acetylcholine releaser/norepinephrine reuptake inhibitor agent in unit dosage form. In particular embodiments, the formulation comprises such acetylcholine releaser/norepinephrine reuptake inhibitor agent in a unit dosage form suitable for providing an amount of the combination sufficient to treat or reduce the symptoms of sleep apnea or other sleep related breathing disorders in an individual. In some embodiments, the formulation comprises the serotonin reuptake inhibitor/5HT2A serotonin receptor antagonist in combination with a second compound in a unit dosage form. In particular embodiments, the invention provides a formulation comprising an acetylcholine releaser/norepinephrine reuptake inhibitor agent selected from the group consisting of bifemelane and teniloxazine.

In some embodiments, the invention provides a formulation comprising an acetylcholinesterase inhibitor agent in combination with a second compound selected from the group consisting of alpha-1 agonists, alpha-2 antagonists, selective serotonin reuptake inhibitors, monoamine oxidase-B inhibitors, cannabinoid agonists, 5HT3 antagonists, 5HT2A antagonists, serotonin reuptake inhibitor/5HT3 antagonists, and serotonin reuptake inhibitor/5HT2A antagonists. In some particular embodiments, the invention provides a formulation comprising an acetylcholinesterase inhibitor agent in combination with a second compound selected from the group consisting of reboxetine, tomoxetine, bicifadine, milnacipran, venlafaxine, desipramine, duloxetine, sertraline and citalopram, fipamezole, dexeferoxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

NN. Formulation Comprising a 5HT3 Antagonist and a Second Compound. In some embodiments, the invention provides a formulation comprising a combination of a first compound having 5HT3 antagonist activity and a second compound selected from the group consisting of selective serotonin reuptake inhibitors, alpha-1 agonists, alpha-2 antagonists, norepinephrine reuptake inhibitors, norepinephrine/serotonin reuptake inhibitors, monoamine oxidase-B inhibitors, acetylcholinesterase inhibitors and cannabinoid agonists. In particular embodiments, the invention provides a formulation in a unit dosage form suitable for providing an amount of the combination sufficient to treat or reduce the symptoms of sleep apnea or other sleep related breathing disorders in an individual. In some embodiments, the formulation comprises the first compound having 5HT3 antagonist activity and the second compound selected from the group consisting of selective serotonin reuptake inhibitors, alpha-1 agonists, alpha-2 antagonists, norepinephrine reuptake inhibitors, norepinephrine/serotonin reuptake inhibitors, monoamine oxidase-B inhibitors, acetylcholinesterase inhibitors and cannabinoid agonists in combination with a third compound in a unit dosage form. In some embodiments, the first compound having 5HT3 antagonist activity is selected from the group consisting of ondansetron, dolasetron, granisetron, tropisetron, ramosetron, and palonosetron.

In some particular embodiments, the invention provides a formulation comprising a first compound having 5HT3 antagonist activity and a second compound selected from the group consisting of reboxetine, tomoxetine, bicifadine, milnacipran, venlafaxine, desipramine, duloxetine, sertraline and citalopram, fipamezole, dexeferoxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491; pargyline, lazabemide and mofegiline.

OO. Formulation of a 5HT2A Antagonist and a Second Compound. In some embodiments, the invention provides a formulation comprising a combination of a first compound having 5HT2A antagonist activity and a second compound selected from the group consisting of 5HT3 antagonists, selective serotonin reuptake inhibitors, alpha-1 agonists, alpha-2 antagonists, norepinephrine reuptake inhibitors, norepinephrine/serotonin reuptake inhibitors, monoamine oxidase-B inhibitors, acetylcholinesterase inhibitors, and cannabinoid agonists. In some embodiments, the invention provides a formulation comprising a combination of a first compound having 5HT3 antagonist activity and a second compound selected from the group consisting of selective serotonin reuptake inhibitors, alpha-1 agonists, alpha-2 antagonists, norepinephrine reuptake inhibitors, norepinephrine/serotonin reuptake inhibitors, monoamine oxidase-B inhibitors, acetylcholinesterase inhibitors and cannabinoid agonists. In particular embodiments, the invention provides a formulation in a unit dosage form suitable for providing an amount of the combination sufficient to treat or reduce the symptoms of sleep apnea or other sleep related breathing disorders in an individual. In some embodiments, the formulation comprises the first compound having 5HT3 antagonist activity and the second compound selected from the group consisting of selective serotonin reuptake inhibitors, alpha-1 agonists, alpha-2 antagonists, norepinephrine reuptake inhibitors, norepinephrine/serotonin reuptake inhibitors, monoamine oxidase-B inhibitors, acetylcholinesterase inhibitors and cannabinoid agonists in combination with a third compound in a unit dosage form. In particular embodiments, the invention provides a formulation in which the compound having 5HT2A antagonist activity is selected from the group consisting of MDL 100907, EMR-62218, and eplivanserin.

PP. Formulation Comprising An Acetylcholinesterase Inhibitor/Serotonin Reuptake Inhibitor. In some embodiments, the invention provides a formulation comprising an agent that has acetylcholinesterase inhibitor activity and serotonin reuptake inhibitor activity. In some such embodiments, the agent having acetylcholinesterase inhibitor activity and serotonin reuptake inhibitor activity is RS-1259. In particular embodiments, the a formulation comprising an agent that has acetylcholinesterase inhibitor activity and serotonin reuptake inhibitor activity further comprises a second compound selected from the group consisting of norepinephrine reuptake inhibitors, norepinephrine/serotonin reuptake inhibitors alpha-1 agonists, alpha-2 antagonists, monoamine oxidase-B inhibitors, 5HT2A antagonists, 5HT3 antagonists, and cannabinoid agonists. In particular embodiments, the second compound is selected from the group consisting of reboxetine, tomoxetine, bicifadine, milnacipran, venlafaxine, desipramine, duloxetine, sertraline and citalopram, fipamezole, dexeferoxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

In some particular embodiments, the invention provides a formulation comprising a first compound that is RS-1259 and a second compound selected from the group consisting of reboxetine, tomoxetine, bicifadine, milnacipran, venlafaxine, desipramine, duloxetine, sertraline and citalopram, fipamezole, dexeferoxan, SDZ-NVI-085, donepezil, galantamine, tetrahydroaminoacridine, rivastigmine, Delta-9-THC, Delta-8-THC, CP 55940, HU-210, WIN55212-2, O-1057, anandamide, and methanandamide, 2-arachidonyl glycerol, ACEA, ACPA, litoxetine, LY628535, lubazodone, R-fluoxetine, ondansetron, dolasetron, granisetron, tropisetron, ramosetron, palonosetron, MDL 100907, EMR-62218, eplivanserin, deprenyl, Ro16-6491, pargyline, lazabemide and mofegiline.

QQ. Formulation of an Agent Having 5HT2/5HT3 Serotonin Receptor Antagonist and Alpha-2 Adrenergic Receptor Antagonist Activity. In some embodiments, the invention provides a formulation comprising a compound having 5HT2/5HT3 serotonin receptor antagonist and alpha-2 adrenergic receptor antagonist activity. In particular embodiments, the formulation comprises an agent having 5HT2/5HT3 serotonin receptor antagonist and alpha-2 adrenergic receptor antagonist activity, which is selected from the group consisting of mirtazapine.

In some particular embodiments of the invention, the formulation comprises a first compound, which is an agent having 5HT2/5HT3 serotonin receptor antagonist and alpha-2 adrenergic receptor antagonist activity, and a second compound, which is selected from the group consisting of acetylcholinesterase inhibitors, selective serotonin reuptake inhibitors, norepinephrine reuptake inhibitors, norepinephrine/serotonin reuptake inhibitors, alpha-1 adrenergic receptor agonists, acetylcholinesterase inhibitors/serotonin reuptake inhibitor agents, acetylcholine releaser/norepinephrine reuptake inhibitor agents, monoamine oxidase-B inhibitors, and cannabinoid agonists.

RR. Formulation of an Ergot Alkaloid with Serotonin Receptor Agonist Activity. In some embodiments, the invention provides a formulation comprising an ergot alkaloid having serotonin receptor agonist activity. In particular embodiments, the ergot alkaloid agonizes one or more serotonin receptors selected from the group consisting of 5HT1A, 5HT1D, and 5HT2A. In other particular embodiments, the ergot alkaloid is selected from the group consisting of ergotamine, dihydroergotamine, acetergamine, brazergoline, bromerguride, cianergoline, delorgotrile, disulergine, ergonovine maleate, etisulergine, lergotrile, lysergide, mesulergine, metergoline, metergotamine, nicergoline, pergolide, propisergide, proterguride, and terguride.

SS. Formulation of an Alpha-1 Adrenergic Receptor Agonist. In some embodiments, the invention provides a formulation for treating or reducing the symptoms of a condition associated with sleep related breathing disorders in an individual comprising an effective amount of an alpha-1 adrenergic receptor agonist. In some such embodiments, the condition associated with sleep related breathing disorders is excessive daytime sleepiness. In particular embodiments, the formulation further comprises a compound that improves the hypoxic index selected from the group consisting of a 5HT1A sertotonin receptor agonist/serotonin reuptake inhibitor/5HT2A/2C serotonin receptor antagonist, selective serotonin reuptake inhibitor /5HT1A serotonin receptor partial agonists, norepinephrine reuptake inhibitor/5HT3 serotonin receptor antagonists, melatonin agonists, selective reuptake inhibitor/5HT3 serotonin receptor antagonists, selective reuptake inhibitor/5HT2A receptor antagonists, acetylcholine releaser/norepinephrine reuptake agents, 5HT2A serotonin receptor antagonists, and acetylcholinesterase inhibitor/serotonin reuptake inhibitor agents, and 5HT2/5HT3 antagonist/alpha-2 antagonists. In some such embodiments, the alpha-1 adrenergic receptor agonist is selected from the group consisting of SDZ-NVI-085, modafinil and adrafinil. An effective daily dose of modafinil is in the range of about 100 to about 400 mg per day, which may be divided into two doses to be administered in the morning and at noon.

TT. Additional Formulations. In some embodiments, the invention provides a formulation for reducing the side effects of a compound administered to improve the hypoxic index in an individual to treat or reduce the symptoms of sleep apnea or other sleep related breathing disorders selected from the group consisting of 5HT1A sertotonin receptor agonist/serotonin reuptake inhibitor/5HT2A/2C serotonin receptor antagonist, selective serotonin reuptake inhibitor/5HT1A serotonin receptor partial agonists, norepinephrine reuptake inhibitor/5HT3 serotonin receptor antagonists, melatonin agonists, selective reuptake inhibitor/5HT3 serotonin receptor antagonists, selective reuptake inhibitor/5HT2A receptor antagonists, acetylcholine releaser/norepinephrine reuptake agents, 5HT2A serotonin receptor antagonists, and acetylcholinesterase inhibitor/serotonin reuptake inhibitor agents, and 5HT2/5HT3 antagonist/alpha-2 antagonists comprising an effective amount of an alpha-1 adrenergic receptor agonist. In some such embodiments, the alpha-1 adrenergic receptor agonist is selected from the group consisting of SDZ-NVI-085, modafinil and adrafinil. An effective dose of modafinil is in the range of about 100 to about 400 mg per day, which may be divided into two doses, one given in the morning and one at noon. In other such embodiments, the norepinephrine reuptake inhibitor, when present, is selected from the group consisting of atomoxetine, reboxetine, tomoxetine, and bicifadine and the norepinephrine/serotonin reuptake inhibitor, when present, is selected from the group consisting of milnacipran, venlafaxine, desipramine and duloxetine.

In some embodiments, the invention provides a formulation for the treatment of sleep related breathing disorders, excessive daytime sleepiness and weight gain comprising an immediate release formulation of a 5HT2/5HT3 antagonist/alpha-2 antagonist and a delayed release formulation of a compound selected from the group consisting of a quinacrine derivative (including quinacrine), norepinephrine reuptake inhibitor/dopamine reuptake inhibitor compound, norepinephrine reuptake inhibitor, norepinephrine/serotonin reuptake inhibitor, dopamine D1 receptor agonist, 5HT1A serotonin receptor agonist, carbonic anhydrase inhibitor, and dopamine-releasing compound. In some such embodiments, the 5HT2/5HT3 antagonist/alpha-2 antagonist, when present, is selected from the group consisting of mirtazapine.

In other such embodiments, the norepinephrine reuptake inhibitor/dopamine reuptake inhibitor compound, when present, is bupropion.

In other such embodiments, the invention provides formulations wherein the D1 dopamine receptor agonist, when present, is selected from the group consisting of SKF38393, CY-208-243, dihydrexidine, SKF82958, A77636 and A68930.

In other such embodiments, the 5HT1A serotonin receptor agonist, when present, is selected from the group consisting of buspirone, gepirone, and alnespirone and Org 13011.

In other such embodiments, the carbonic anhydrase inhibitor, when present, is selected from the group consisting of acetazolamide, zonisamide, methazolamide, dichlorphenamide, and topiramate.

In other such embodiments, the dopamine-releasing compound is selected from the group consisting of amantadine, rimantadine, methamphetamine, dextroamphetamine, laevoamphetamine, and methylphenidate.

In some such embodiments, the formulation the quinacrine derivative, when present, is selected from those compounds having the following general formula:

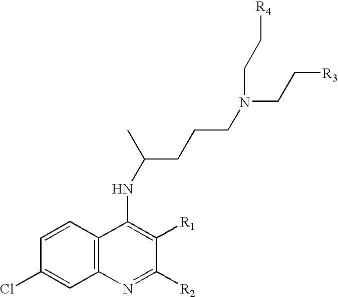

wherein R1 and R2 may be hydrogen, an alkyl group, a hydroxyl group, a methoxy group, a halogen group, an amino group, or together may form a substituted or unsubstituted fused aromatic ring; and

wherein R3 and R4 may be hydrogen, a hydroxy group, alkyl group, methoxy group, or halogen group.

In particular embodiments, the quinacrine derivative, when present is selected from the group consisting of quinacrine, chloroquine, and hydroxychloroquine.

In some embodiments, the invention provides a formulation for reducing one or more symptoms in an individual, which are associated with the use of a 5HT2/5HT3 antagonist/alpha-2 antagonist in the treatment of sleep related breathing disorders, selected from the group consisting of the excessive daytime drowsiness and weight gain comprising an effective amount of a compound selected from the group consisting of quinacrine, norepinephrine reuptake inhibitor/dopamine reuptake inhibitor compound, norepinephrine reuptake inhibitor, norepinephrine/serotonin reuptake inhibitor, dopamine D1 receptor agonist, 5HT1A serotonin receptor agonist, carbonic anhydrase inhibitor, and dopamine-releasing compound. In some such embodiments, the 5HT2/5HT3 antagonist/alpha-2 antagonist, when present, is selected from the group consisting of mirtazapine. In other such embodiments, the norepinephrine reuptake inhibitor/dopamine reuptake inhibitor compound, when present, is bupropion. In other such embodiments, the norepinephrine reuptake inhibitor is selected from the group consisting of atomoxetine, reboxetine, tomoxetine, and bicifadine and the norepinephrine/serotonin reuptake inhibitor is selected from the group consisting of milnacipran, venlafaxine, desipramine and duloxetine. In other such embodiments, the invention provides formulations wherein the D1 dopamine receptor agonist, when present, is selected from the group consisting of SKF38393, CY-208-243, dihydrexidine, SKF82958, A77636 and A68930.

In other such embodiments, the invention provides formulations wherein the D1 dopamine receptor agonist, when present, is selected from the group consisting of SKF38393, CY-208-243, dihydrexidine, SKF82958, A77636 and A68930.

In other such embodiments, the 5HT1A serotonin receptor agonist, when present, is selected from the group consisting of buspirone, gepirone, and alnespirone and Org 13011.

In other such embodiments, the carbonic anhydrase inhibitor, when present, is selected from the group consisting of acetazolarnide, zonisamide, methazolamide, dichlorphenamide, and topiramate.

In other such embodiments, the dopamine-releasing compound is selected from the group consisting of amantadine, rimantadine, methamphetamine, dextroamphetamine, laevoamphetamine, and methylphenidate.

In some such embodiments, the formulation the quinacrine derivative, when present, is selected from those compounds having the following general formula:

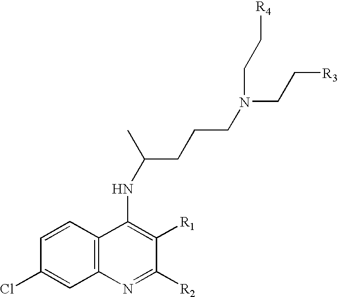

wherein R1 and R2 may be hydrogen, an alkyl group, a hydroxyl group, a methoxy group, a halogen group, an amino group, or together may form a substituted or unsubstituted fused aromatic ring; and

wherein R3 and R4 may be hydrogen, a hydroxy group, alkyl group, methoxy group, or halogen group.

In particular embodiments, the quinacrine derivative, when present is selected from the group consisting of quinacrine, chloroquine, and hydroxychloroquine.

UU. Formulation for Reducing Pressure During CPAP. A formulation for reducing the pressure applied during continuous positive airway pressure therapy in the treatment of sleep related breathing disorders in an individual comprising an effective amount of one or more compounds that suppress REM sleep, increase deep slow wave sleep, and increase the tone of upper airway muscles during sleep and/or increase respiratory drive. In some such embodiments, the invention provides a formulation comprising a compound selected from the group of compounds consisting of a quinacrine derivative (including quinacrine), acetylcholinesterase inhibitors, cannabinoid agonists, 5HT1A sertotonin receptor agonist/serotonin reuptake inhibitor/5HT2A/2C serotonin receptor antagonists, alpha-2 adrenergic receptor antagonists, 5-HT1A serotonin receptor agonists, 5HT2A/2C serotonin receptor agonists, melatonin agonists, 5HT3 serotonin receptor antagonists, 5HT2A serotonin receptor antagonists, selective serotonin reuptake inhibitors, norepinephrine reuptake inhibitors, alpha-1 adrenergic receptor agonists, monoamine oxidase (MAO)-B inhibitors, ergot alkaloids, 5HT2B/2C serotonin receptor antagonists, serotonin reuptake inhibitor/5HT3 serotonin receptor antagonists, serotonin reuptake inhibitor/5HT2A serotonin receptor antagonists, acetylcholine releaser/norepinephrine reuptake inhibitor agents, norepinephrine reuptake inhibitor/5-HT3 serotonin receptor antagonists, selective serotonin reuptake inhibitor/5HT1A serotonin receptor partial agonists, acetylcholinesterase inhibitor/serotonin reuptake inhibitor agents, 5HT2 antagonist/alpha-1 agonists, 5HT2/5HT3 serotonin receptor antagonist/alpha-2 adrenergic receptor antagonists, and norepinephrine reuptake inhibitor/dopamine reuptake inhibitor compounds.

In some such embodiments, the formulation the quinacrine derivative, when present, is selected from those compounds having the following general formula:

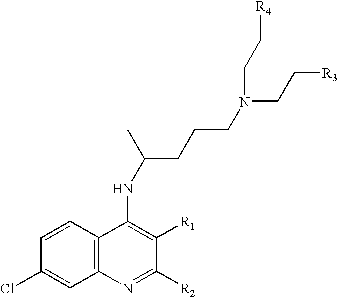

wherein R1 and R2 may be hydrogen, an alkyl group, a hydroxyl group, a methoxy group, a halogen group, an amino group, or together may form a substituted or unsubstituted fused aromatic ring; and

wherein R3 and R4 may be hydrogen, a hydroxy group, alkyl group, methoxy group, or halogen group.

In specific embodiments, the quinacrine derivative, when present, is selected from the group consisting of quinacrine, chloroquine, hydroxychloroquine and combinations thereof.

VV. Formulations of Specific Combinations.

In other embodiments, the formulation comprises a combination of two drugs, wherein the drug combination is selected from the group of combinations consisting of 5HT2/5HT3 antagonist/alpha-2 antagonists and norepinephrine reuptake inhibitor/dopamine reuptake inhibitor compounds, 5HT3 antagonists and 5HT2A antagonists, 5HT3 antagonists and serotonin reuptake inhibitor/5HT2A antagonists, 5HT3 antagonists, 5HT1A sertotonin receptor agonist/serotonin reuptake inhibitor/5HT2A/2C serotonin receptor antagonists, and 5HT3 antagonists and 5HT2 antagonist/alpha-1 agonists.

Other specific combinations within the present invention include: mirtazapine and zonisamide; and milnacipran and zonisamide.

WW. Nasal Formulations. In particular embodiments, the invention provides a nasal formulation for the treatment of sleep related breathing disorders. The formulation comprises an effective amount of a corticosteroid in combination with a compound selected from the group of compounds consisting of quinacrine, acetylcholinesterase inhibitors, cannabinoid agonists, 5HT1A sertotonin receptor agonist/serotonin reuptake inhibitor/5HT2A/2C serotonin receptor antagonists, alpha-2 adrenergic receptor antagonists, 5-HT1A serotonin receptor agonists, 5HT2A/2C serotonin receptor agonists, melatonin agonists, 5HT3 serotonin receptor antagonists, 5HT2A serotonin receptor antagonists, selective serotonin reuptake inhibitors, norepinephrine reuptake inhibitors, norepinephrine/serotonin reuptake inhibitors, alpha-1 adrenergic receptor agonists, monoamine oxidase (MAO)-B inhibitors, ergot alkaloids, dopamine D1 receptor agonists, carbonic anhydrase inhibitors, 5HT2B/2C serotonin receptor antagonists, serotonin reuptake inhibitor/5HT3 serotonin receptor antagonists, serotonin reuptake inhibitor/5HT2A serotonin receptor antagonists, acetylcholine releaser/norepinephrine reuptake inhibitor agents, norepinephrine reuptake inhibitor/5-HT3 serotonin receptor antagonists, selective serotonin reuptake inhibitor/5HT1A serotonin receptor partial agonists, acetylcholinesterase inhibitor/serotonin reuptake inhibitor agents, 5HT2 antagonist/alpha-1 agonists, 5HT2/5HT3 serotonin receptor antagonist/alpha-2 adrenergic receptor antagonists, and norepinephrine reuptake inhibitor/dopamine reuptake inhibitor.

In some such embodiments, the corticosteroid, when present, is selected from the group consisting of hydrocortisone, cortisone, prednisone, and dexamethasone.

In some such embodiments, the formulation the quinacrine derivative, when present, is selected from those compounds having the following general formula:

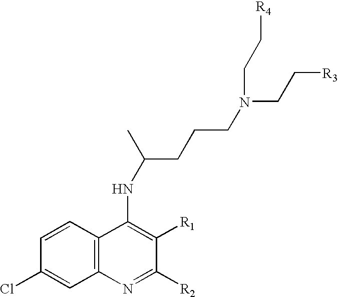

wherein R1 and R2 may be hydrogen, an alkyl group, a hydroxyl group, a methoxy group, a halogen group, an amino group, or together may form a substituted or unsubstituted fused aromatic ring; and

wherein R3 and R4 may be hydrogen, a hydroxy group, alkyl group, methoxy group, or halogen group.

In particular embodiments, the quinacrine derivative, when present is selected from the group consisting of quinacrine, chloroquine, and hydroxychloroquine.

XX. Formulation of a Carbonic Anhydrase Inhibitor. In some embodiments, the invention provides a formulation comprising at least one carbonic anhydrase inhibitor. In some such embodiments, the formulation comprising at least one carbonic anhydrase inhibitor further comprises a second compound selected from the group consisting of 5HT1A serotonin receptor agonist/serotonin reuptake inhibitor/5HT2A/2C serotonin receptor antagonists, selective serotonin reuptake inhibitors, norepinephrine/serotonin reuptake inhibitors, norepinephrine reuptake inhibitors, dopamine/serotonin receptor antagonists, norepinephrine reuptake inhibitor/5HT3 serotonin receptor antagonists, serotonin reuptake inhibitor/5HT3 serotonin reuptake inhibitor antagonists, and selective reuptake inhibitor/5HT2A serotonin receptor antagonists.

In particular embodiments, the invention provides a formulation comprising at least one carbonic anhydrase inhibitor, wherein the carbonic anhydrase inhibitor is acetazolamide, zonisamide, methazolamide, dichlorphenamide, and topiramate. In particular, zonisamide and topiramate are considered most advantageous in the context of this invention.

AAA. Methods of Administering an HI Histamine Receptor Antagonist/5HT2 Serotonin Receptor Antagonist. In some embodiments, the invention provides a method of treating or reducing the symptoms of sleep apnea or other sleep related breathing disorders in an individual, the method comprising administering an effective amount of one or more compounds that suppress REM sleep, increase deep slow wave sleep, and increase the tone of upper airway muscles during sleep and/or increase respiratory drive, further wherein at least one compound that suppresses REM sleep, increases deep slow wave sleep, increases tone of upper airway muscles during sleep and/or increases respiratory drive is an H1 histamine receptor antagonist/5HT2 serotonin receptor antagonist. In particular embodiments, the H1 histamine receptor antagonist/5HT2 serotonin receptor antagonist is cyproheptadine. In some such embodiments, the method further comprises administering a second compound selected from the group consisting of selective serotonin reuptake inhibitors, norepinephrine reuptake inhibitors, norepinephrine/serotonin reuptake inhibitors, carbonic anhydrase inhibitors, selective serotonin reuptake inhibitor/5HT1A serotonin receptor partial agonists, and serotonin reuptake inhibitor/5HT3 serotonin receptor antagonists. In particular embodiments, the second compound is selected from the group consisting of sertraline, citalopram, atomoxetine, reboxetine, tomoxetine, bicifadine, milnacipran, venlafaxine, desipramine, duloxetine, zonisamide, vilazodone, and litoxetine.

The invention further comprises a formulation comprising an H1 histamine receptor antagonist/5HT2 serotonin receptor antagonist. In particular, the invention provides a formulation wherein the H1 histamine receptor antagonist/5HT2 serotonin receptor antagonist is cyproheptadine. More particularly, the invention provides a formulation comprising an H1 histamine receptor antagonist/5HT2 serotonin receptor antagonist and a second compound selected from the group consisting of selective serotonin reuptake inhibitors, norepinephrine reuptake inhibitors, norepinephrine/serotonin reuptake inhibitors, carbonic anhydrase inhibitors, selective serotonin reuptake inhibitor/5HT1A serotonin receptor partial agonists, and serotonin reuptake inhibitor/5HT3 serotonin receptor antagonists. In particular embodiments, the invention provides such a formulation in which the second compound is selected from the group consisting of sertraline, citalopram, atomoxetine, reboxetine, tomoxetine, bicifadine, milnacipran, venlafaxine, desipramine, duloxetine, zonisamide, vilazodone, and litoxetine.

### EXAMPLE

Example 1

Protocol for Determining the Effects of Mirtazapine and Zonisamide on Upper Airway Tone in Rats. The effects of mirtazapine and zonisamide on upper airway muscle tone during sleep are assessed in a rat model to evaluate the potential efficacy of these agents in humans. Mirtazapine and zonisamide, alone and in combination, are given to conscious adult rats and their sleep related behavior is recorded for four to six hours. To record sleep related activity, the animals have skull electrodes and respiratory muscle activity electrodes implanted. Electrode instrumentation takes place while the animals are anesthetized. Instrumentation includes an incision on the top of the head to expose the skull and the placement of three small screws into the top of the skull. Electroencephalography (EEG) electrodes are attached to the screws for determination of sleep stages. In addition, a total of three pairs of thin, flexible, wire electrodes for measuring electromyography (EMG) activity are placed into 1) the back of the tongue (genioglossus muscle, a primary muscle controlling upper airway tone), 2) the muscles of the back of the neck and 3) the main muscle activated during breathing, the diaphragm. The thin wire electrodes in the tongue are inserted through a small incision under the jaw and are brought under the skin to the back of the neck. The diaphragm electrodes are then inserted through a small incision in the skin and tunneled under the skin to the back of the neck. All wire electrodes and head screws are inserted into a platform on the top of the head. The platform is fixed in place with dental acrylic on the top of the animal's head. All incisions are then closed with needle and suture thread.

Animals are given drugs to reduce pain and inflammation associated with the surgery. Following surgery the animals are allowed a recovery period and are monitored for one week before receiving any drug injections. Drug injections are performed intraperitoneally at the following dose ranges: 0.1-10 mg/kg for mirtazapine and 10-100 mg/kg for zonisamide. Effects of both mirtazapine and zonisamide on EMG activity in the genioglossus muscle and diaphragm during specific sleep stages are measured. Additive effects of mirtazapine and zonisamide combinations on EMG activity are also assessed.

The results of the foregoing experiment demonstrate the potential effectiveness of mirtazapine, either as a monotherapeutic agent or in combination with zonisamide, in the treatment of sleep related breathing disorders, such as sleep apnea and snoring.

### Example 2

Mirtazapine Monotherapy. A proof-of-concept study is designed to demonstrate the effects of mirtazapine on obstructive sleep apnea (OSA). The study includes dosing at multiple dosage levels over a period of time. In particular, the study is a six week single-blind cross-over design study in patients who have been diagnosed with OSA. The study patients must have an Apnoea-Hypopnoea Index (AHI) of between 10 and 40, an age of at least 21 years, and a calculated body mass index (BMI) equal to or less than 40 at the time of study entry. The patients are randomly assigned to one of six dose-sequence groups in a ratio of 1:1:1:1:1:1. Thus, the total number of patients is 42—six in each dose-sequence group; those who terminate early from the study are replaced.

The criteria for inclusion in the study are as follows: (1) Patients must be capable of giving informed consent; (2) Patients must have an AHI of 10-40; (3) Patients must be at least 21 years of age; (4) Each patient must have a BMI of no more than 40; (5) Current CPAP users are excluded (patients with no more than 3 days of CPAP use in the last 12 months are allowed); (6) Patients must be non-smokers with no history of smoking for at least two years; (7) Each patient must have a baseline Epworth Sleep Scale of greater than 10; (8) Patients must be able to read and speak English; (9) Females must be either postmenopausal (no menses for at least 1 year) or status-post hysterectomy or bilateral oophorectomy or, if of childbearing potential, must have a negative urine pregnancy test prior to randomization and be using a medically acceptable form of contraception (e.g., hormonal birth control, intrauterine device, double barrier (male condom, female condom, diaphragm) or a barrier method plus a spermicidal agent (contraceptive foam, jelly or cream)).

The criteria for exclusion from the study include: (1) Clinically significant comorbidity, including any unstable cardiovascular, gastrointestinal, metabolic, pulmonary (e.g., asthma, COPD), renal, neurological, hepatic, hematologic, immunologic, endocrine, and/or neoplastic disease based on Principal Investigator judgment; (2) Hypertension (those patients with controlled hypertension-systolic <=140 and diastolic <=90—for at least 3 months prior to Tx0/Baseline are allowed to enrol); (3) Patients with evidence of active liver disease (levels of AST, ALT and/or alkaline phosphatase >2× the upper limit of the normal range (ULN) for the laboratory performing the test; (4) Patients with a white blood count below normal range or a count >1.5× the ULN; (5) Patients with anaemia as defined by haemoglobin <80% lower limit of normal; (6) Patients with impaired renal function as evidenced by a creatine value >1.2×ULN; (7) Severe craniofacial abnormalities; (8) Concomitant use of any stimulant medications, including modafinil; (9) Concomitant use sedative hypnotics, tranquilizers, antihistamines (non-sedating antihistamines are allowed), benzodiazepines, or clonidine; (10) Concomitant use of any anticonvulsant medication; (11) Current diagnosis of any psychiatric illness including any psychotic, schizoaffective, and/or major affective disorder(s) based on DSM-IV criteria; (12) Patients who are receiving concomitant therapy with MAO-A or -B inhibitors, tricyclics, tetracyclics, SSRI agents, NARI agents, SNRI agents, alpha-agonists or St. John's Wort; (13) Current diagnosis of any substance abuse disorder based on DSM-IV criteria; (14) Previous or current history of a generalized or partial seizure disorder, or current treatment for any form of seizure disorder; (15) Pregnant or lactating females; (16) Concomitant use of drugs having known Cytochrome P450 induction or inhibition properties.

The daily doses of mirtazapine in the study are 0, 7.5, 15, 30, and 45 mg/day. The six dose-sequence groups are as follows:

Dose-Sequence Group 1: Tx0/Baseline: begin 0 mg mirtazapine and continue 14 days; Tx2/Day 15: dose increase to 15 mg p.o. nocte mirtazapine for two weeks; Tx4/Day 29: dose increase to 45 mg p.o. nocte mirtazapine for two weeks; Tx6/Day 43 (end of study) final evaluations are performed.

Dose-Sequence Group 2: Tx0/Baseline: begin 15 mg mirtazapine and continue 14 days; Tx2/Day 15: dose increase to 30 mg p.o. nocte mirtazapine for two weeks; Tx4/Day 29: dose decreased to o mg p.o. nocte mirtazapine for two weeks; Tx6/Day 43 (end of study) final evaluations are performed.

Dose-Sequence Group 3: Tx0/Baseline: begin 7.5 mg mirtazapine and continue 14 days; Tx2/Day 15: dose increase to 45 mg p.o. nocte mirtazapine for two weeks; Tx4/Day 29: dose decrease to 30 mg p.o. nocte mirtazapine for two weeks; Tx6/Day 43 (end of study) final evaluations are performed.

Dose-Sequence Group 4: Tx0/Baseline: begin 0 mg mirtazapine and continue 14 days; Tx2/Day 15: dose increase to 7.5 mg p.o. nocte mirtazapine for two weeks; Tx4/Day 29: dose increase to 30 mg p.o. nocte mirtazapine for two weeks; Tx6/Day 43 (end of study) final evaluations are performed.

Dose-Sequence Group 5: Tx0/Baseline: begin 7.5 mg mirtazapine and continue 14 days; Tx2/Day 15: dose increase to 30 mg p.o. nocte mirtazapine for two weeks; Tx4/Day 29: dose decrease to 15 mg p.o. nocte mirtazapine for two weeks; Tx6/Day 43 (end of study) final evaluations are performed.

Dose-Sequence Group 6: Tx0/Baseline: begin 15 mg mirtazapine and continue 14 days; Tx2/Day 15: dose increase to 45 mg p.o. nocte mirtazapine for two weeks; Tx4/Day 29: dose decrease to 7.5 mg p.o. nocte mirtazapine for two weeks; Tx6/Day 43 (end of study) final evaluations are performed.

The patients are evaluated on the following efficacy criteria: Calculated change in AHI score from baseline to follow-up; change in Epworth Sleep Scale (ESS) score; change in Patient Global Impression of Change (PGIC); change in AusEd driving simulator performance; change in SF-36 score (total and subdomain), change in FOSQ score; change in Karolinska Sleep Scale (KSS) score. In addition, patients are evaluated based on the following safety criteria: Medical history; vital signs (height, weight, heart rate, respiration, blood pressure (standing and sitting); urine pregnancy test for females of childbearing potential; severity and relationship of adverse events to study drugs; death and other serious adverse events; discontinuations due to adverse events.

The timing of treatments and procedures during the study are conducted as indicated in Table 1, following:

Timing of Treatments and Procedures

TABLE 1
Timing of Treatments and Procedures

| | | | | | | | T × 6/Day |
|---|---|---|---|---|---|---|---|
| | | Day 3 | | Day 21 | | Day 35 | 43 |
| | | (±1 day) | T × 2/Day | (±1 day) | T × 4/Day | (±1 day) | Early |
| | | Telephone | 15 | Telephone | 29 | Telephone | Termination |
| | T × 0/ | Contact | (−2/+4 | Contact | (−2/+4 | Contact | (−2/+4 |
| Procedure | Baseline | Only | days) | Only | days) | Only | days) |
| Informed consent | X | | | | | | |
| Inclusion/exclusion | X | | | | | | |
| criteria | | | | | | | |
| Medical history | X | | | | | | |
| Blood draw for | X | | X | | X | | X |
| diagnostic | | | | | | | |
| laboratories | | | | | | | |
| (metabolic/liver | | | | | | | |
| profile, CBC, | | | | | | | |
| kidney function)^{e} | | | | | | | |
| Vital signs, height^{a}, | X | | X | | X | | X |
| weight | | | | | | | |
| Pregnancy test^{b} | X | | | | | | |
| Diagnostic Sleep | X | | X | | X | | X |
| Laboratory | | | | | | | |
| Assessment^{c} | | | | | | | |
| Dispense home | X | | X | | X | | |
| breathing | | | | | | | |
| monitoring device^{d} | | | | | | | |
| Modified Epworth | X | | X | | X | | X |
| Sleep Scale | | | | | | | |
| Karolinska Sleep | X | | X | | X | | X |
| Scale | | | | | | | |
| AusEd Simulator | X | | X | | X | | X |
| test^{f} | | | | | | | |
| SF36 | X | | | | | | X |
| FOSQ | X | | | | | | X |
| PGIC | | | X | | X | | X |
| Concomitant | X | X | X | X | X | X | X |
| medications | | | | | | | |
| Adverse events | | X | X | X | X | X | X |
| Drug Dispensation | X | | X | | X | | |
| Drug Accountability | | | X | | X | | X |

^{a}Height is measured only at screening.
^{b}Urine Pregnancy tests are done only on females of childbearing potential (i.e., females who are not surgically sterile or are <2 years postmenopausal).
^{c}Sleep Lab results from testing within 4 weeks prior to the Baseline visit are allowed for use as the Baseline Sleep Lab requirement, provided that BMI has not changed by more than 1 unit.
^{d}A home breathing monitoring device is dispensed to the patient at T × 0, T × 2 and T × 4. Used devices are collected by the site at T × 2, T × 4 and T × 6.
^{e}Applicable Clinical Laboratory results within 2 weeks prior to the Baseline visit are allowed for use as the Baseline Clinical Laboratory Results
^{f}The AusEd driving simulator must be performed in the morning; preferably following the PSG (or use of home breathing monitoring device, if PSG is not performed at baseline visit).

Study Procedures

Timing of Procedures

Patient enrolment and baseline procedures: (1) Patients sign the informed consent. (2) The physician reviews the medical history of the patient. (3) The physician reviews the admission criteria to determine that the patient is appropriate for the study. (4) Clinical laboratory blood samples are drawn. Patients that have had the applicable blood samples drawn within 2 weeks of signing the informed consent do not need to have blood samples redrawn. The physician must assure that clinical laboratory results are reviewed prior to drug dispensation. (5) A sleep laboratory evaluation is scheduled unless the patient has had a sleep laboratory diagnosis within the previous 4 weeks. (6) Vital signs are recorded (7) The patients complete study questionnaires. (8) Patients are trained on the AusEd driving simulator the morning following the baseline sleep laboratory, or, if a baseline sleep laboratory assessment is not necessary, in the morning. (10) Patients complete the AusEd driving simulator the morning following the baseline sleep laboratory, or, if a baseline sleep laboratory assessment is not necessary, in the morning. (11) Study personnel review subject AHI and determine if the subject meets criteria for mild, moderate or severe sleep apnea. (12) The physician ensures that the subject continues to meet admission criteria. (13) The patient is trained in the use of the breathing monitoring device. (14) The patient is assigned to a dose-sequence group. (15) A home monitoring device is dispensed to the patient (this device must be used by the patient in their home and returned to the site the next day prior to drug dispensation). (16) Drug is dispensed.

On-Study Procedures, Tx2/Day 15 and Tx4/Day 29: (1) Patients return to the clinic in the evening. (2) Patients return their home monitoring devices for data retrieval. (3) Clinical laboratory samples are drawn. (4) Vital signs are collected. (5) Adverse events and concomitant medication use are assessed. (6) Patient questionnaires are completed. (7) A sleep laboratory evaluation is conducted. (8) The AusEd driving simulator task is completed the following morning. (9) Two home monitoring devices are dispensed to the patient. (10) Drug is dispensed to the patient the following morning.

Telephone Contacts, Study Days 3, 21, and 35: (1) Patients are contacted at study Days 3, 21, and 35 to assess adverse events, review concomitant medications and home device usage.

Exit Evaluation: At the patient's final study visit (Tx6/Day 43 or Early Termination), the following procedures are performed: (1) Patients return to the clinic in the evening. (2) Patients return their home monitoring devices for data retrieval. (3) Clinical laboratory samples are drawn. (4) Vital signs are collected. (5) Adverse events and concomitant medication use is assessed. (6) Patient questionnaires are completed. (7) A sleep laboratory evaluation is performed. (8) The AusEd driving simulator task is completed the following morning.

Efficacy assessments—Change in AHI Score: The patient's AHI score is based on results of a diagnostic Sleep Laboratory assessment that is conducted at Tx0/Baseline, Tx2/Day 15, Tx4/Day 29, and Tx6/Day 43 or Early Termination.

Change in score of the Epworth Sleep Scale: Each patient completes the modified Epworth Sleep Scale questionnaire at Tx0/Baseline, Tx2/Day 15, Tx4/Day 29, and Tx6/Day 43 or Early Termination. The questionnaire is designed to evaluate daytime sleepiness.

Change in score of the Karolinska Sleep Scale (KSS): Each patient completes the Karolinska Sleep Scale at Tx0/Baseline, Tx2/Day 15, Tx3/Day 29, and Tx6/Day 43 or Early Termination.

Patient Global Impression of Change (PGIC): Each patient completes an assessment of patient global impression of change at Tx2/Day 15, Tx4/Day 29, and Tx6/Day 43 or Early Termination.

SF-36: Each patient completes the SF-36 questionnaire at Tx0/Baseline and Tx6/Day 43 or Early Termination.

FOSQ (Functional Outcome Sleep of Sleep Questionaire): Each patient completes the FOSQ at Tx0/Baseline and Tx6/Day 43 or Early Termination.

AusEd Driving Simulator: The AusEd driving simulator evolved as a joint research project between the Sleep Units of St Vincents Sleep Disorders Service, Royal North Shore Hospital Sydney, Australia and the Respiratory and Sleep Research Unit at the Royal Infirmary Edinburgh, Scotland. The simulator is computer based. The replication of a usual rural road at night allows for the assessment of reaction times through a braking exercise, speed deviation and road deviation. The AusEd is unique as most other simulator programmes have used more stimulating protocols potentially alerting drivers while driving alone.

This is a tool tailored to assess three areas of driving performance. These are tracking error measured by steering accuracy, divided attention task by velocity deviation and reaction time. The simulator is installed on a PC with windows NT operating system, with a 21″ computer screen, a Thrustmaster T2 steering wheel and pedals (Hillsboro, Oreg., USA) and computer speakers.

Subjects are asked to drive for 30 min, keeping the speed between 60-80 units/hr and to maintain the vehicle in the middle of the left hand lane. Braking reaction time is assessed by 10 trucks appearing intermittently throughout the drive. Like a real vehicle, the simulator is “driven” using a steering wheel, acceleration and brake pedals. The simulated task is designed for monotonous night-time driving on rural road. The visual field is from the driver's seat, looking forward, low beam lighting of a dual carriageway highway. All lighting is turned off during the test sessions. In addition, a low frequency of 60 dB, engine-like noise accompanies the duration of the drive. A standardised driving route is selected based on previous studies in our laboratory, whereby straight section and chicanes or curved parts are 5/7 and 2/7 of the road. This route is used throughout all test sessions. A five-minute practice run is given at the beginning of each test round to minimise practice effect.

The subject controls the simulator by the accelerator, brake pedal and steering wheel. The driving simulator run is on a dual lane highway at night, where forward vision is limited to the equivalent of lights on ‘low beam’. Ten slowly moving trucks going in the same direction as the driver, appear during the 30 minute drive. There are no other vehicles going in either direction. Reflective markers are found on both sides of the road, which is standard in Australia for many rural roads. A 60 dB simulated engine noise of a low continuous frequency is played through the computer speakers throughout the drive time. Each subject has an identical presentation of trucks, area of straight road ( 5/7) and chicanes ( 2/7). In the top left hand corner of the monitor is the speedometer, which is red, indicating that the subjects was either driving >80 kms/per hour or <60 kms/per hour. The background on the screen is black (to simulate night time). The markers at the side of the road and line markers in the centre of the road are white and easily visible.

Ambulatory Sleep Apnoea Monitoring Devices: Two experimental home breathing monitoring devices are evaluated in this study. Patients enrolled in this protocol receive either the ARES or the Flow Wizard at their initial clinic visit. The patients are asked to record between three and five nights per week of sleep breathing data. The Flow Wizard device can be used for up to 3 nights and the ARES device for up to 4 nights of data recording. The patient is then instructed to bring the devices back to the clinic for their Tx4 and Tx6 visits. At the Tx0 visit, patients are dispensed one device, either one Flow Wizard or one ARES and they use it during that night's sleep at home and then return it to the clinic the next day. Upon returning the device to the clinic patients are dispensed their study drug for the first two weeks of their trial participation and they receive 2 devices to take home. At the Tx2 and Tx4 visits they are dispensed two additional devices.

These devices are used to supplement the data obtained from the sleep laboratory diagnostic evaluations.

ARES: The ARES device (Apnoea Risk Evaluation System) is manufactured by B-Alert, Inc. in Carlsbad, Calif., and has been approved by the US FDA for the diagnosis of possible sleep apnoea. This device, which is worn to bed by the patient, mounts on the forehead using an elastic strap. The device includes an optical oximeter than can measure haemoglobin desaturation, an accelerometer that detects position, a microphone for snoring measurement, and a nasal pressure cannula for determining air flow and obstruction.

Flow Wizard: The Flow Wizard recorder is a battery operated device designed to measure upper airway resistance and nasal airflow in patients during periods of sleep. The parameter of interest is the nasal flow and is indirectly measured from the standard nasal cannula (nasal prongs) by measuring the generated pressure. A sensitive pressure transducer picks up small pressure changes in the nose measured by nasal cannula. During inspiration the pressure is lower inside the nose than outside. It works in the opposite way during expiration. The nasal prongs on the cannula provide resistance. The higher the airflow, the larger the pressure difference between inside and outside of the nose. The processed signal is stored in the flash memory unit and loaded on to the nominated computer for analysis and interpretation. Up to three (3) nine hour recordings can be stored in the recorder before data transfer procedures are required.

The Flow Wizard recorder is designed to sit on a bedside table or on the floor beside the bed while a patient is sleeping. The recorder has a luer lock at one end where a standard nasal oxygen cannula is attached and a USB port at the other end for device setup and data transfer procedures. A single tactile button is located on the centre top of the device to enable users to initiate recordings with depression of the button for a five second period. Once a recording is initiated (and the LED is constantly illuminated), the Flow Wizard recorder acquires the signal for a period of nine (9) hours then cease recording. Patients are not required to terminate the recording but can do so if this occurs within the nine hour period.

Laboratory and Vital Sign Assessments. Laboratory and vital sign assessments are performed as described below.

Laboratory Analyses: Blood samples for haematology and clinical chemistry assessment are obtained at Tx0/Baseline, Tx2, Tx4, and Tx6 (study end) or Early Termination. Patients must satisfy all entry criteria prior to entering into the baseline phase of this trial.

Any laboratory parameter that the investigator believes to be clinically significant is recorded as an adverse event. As expected for all adverse events, clinically significant laboratory abnormalities are followed until clinical resolution, improvement or stabilization.

Haematology: Haematology assessments include red blood cell count (RBC), haemoglobin (Hgb), haematocrit (Hct), mean corpuscular volume (MCV), mean corpuscular haemoglobin (MCH), mean corpuscular haemoglobin concentration (MCHC), white blood cell count (WBC) including differential (neutrophils, lymphocytes, monocytes, eosinophils, and basophils), and platelet count. Blood samples obtained at the study site are analysed by the local site laboratory.

Serum Chemistries: Chemistry assessments include sodium, potassium, chloride, carbon dioxide (CO2), calcium, phosphorus, glucose, blood urea nitrogen (BUN), creatinine, alkaline phosphatase (AP), total bilirubin, direct bilirubin, aspartate aminotransferase (AST), alanine aminotransferase (ALT), lactate dehydrogenase (LDH), total protein, albumin, triglyceride, cholesterol, and uric acid. Blood samples obtained at the study site are analysed by the local site laboratory.

Pregnancy Test: A urine β-human chorionic gonadotropin hormone sample for pregnancy testing is performed by the site, using an in-office test kit at Tx0/randomization for all females of child bearing potential, as defined by the inclusion criteria.

Vital Signs: Standing and sitting blood pressure and heart rate, weight and temperature are obtained at each clinic visits; height are also obtained at the baseline visit.

Adverse Events: An Adverse Event or Adverse Experience (AE) is any untoward medical occurrence in a patient or clinical investigation subject administered a pharmaceutical product. It is not necessary that the AE have a causal relationship to treatment with the product.

An AE therefore is any unfavourable and unintended sign (for example, a clinically significant abnormal laboratory finding), symptom, disease or worsening in current medical conditions whether or not considered related to study medication.

Adverse findings not considered clinically significant which are related to routine laboratory evaluations, physical exams, or vital signs are not to be recorded on the AE reporting page. They should instead be recorded on the relevant CRF page.

Mirtazapine is purchased commercially. Mirtazapine is available as a white tablet in doses of 15 mg, 30 mg and 45 mg. The doses of 7.5 mg and 15 mg utilized in this study are achieved by cutting in half the 15 and 30 mg tablets respectively. Study drug is taken orally once daily before bedtime (nocte). The required amount of each dose of study drug per patient is packaged in blister packs and secondary packaging is in patient kit form. Drug is dispensed from the patient kit by the pharmacist at each study visit. Study drug is labelled according to Therapeutic Goods Administration requirements for clinical trial supplies.

Mirtazapine as provided is stored at room temperature for the duration of this study with no special provisions.

Concomitant use of any stimulant medication, sedative hypnotics, tranquilizers, antihistamines (non-sedating antihistamines are allowed), benzodiazepines, or clonidine are not permitted during this study. In addition, concomitant therapy with MAO-A or -B inhibitors, tricyclics, tetracyclics, SSRI agents, NARI agents, SNRI agents, St. John's Wort, or alpha-agonists are not permitted during this study.

Patient compliance with the protocol is assessed through pill counting and directed query. Patients are directed to bring their study medication bottles with them to each clinic visit. The study coordinator verifies the pill count remaining, and any discrepancy is discussed with the patient.

Medications that are not specifically listed as excluded in the excluded medication list or table of cytochrome p450 inhibitors and inducers are acceptable as concomitant therapy, at the discretion of the investigator.

The results of this study demonstrate the effectiveness of mirtazapine as a monotherapy in the treatment of sleep related breathing disorders, such as sleep apnea and snoring.

### Example 3

### Mirtazapine and Zonisamide Combination Therapy

A proof-of-concept study is designed to demonstrate the effects of mirtazapine on obstructive sleep apnea (OSA) when used in combination with zonisamide. The study includes dosing with mirtazapine alone, with placebo and with mirtazapine plus zonisamide. In particular, the study is a four week single-blind, randomized study in patients who have been diagnosed with OSA. The study patients must have an Apnoea-Hypopnoea Index (AHI) of between 10 and 40, an age of at least 21 years, and a calculated body mass index (BMI) equal to or less than 34 at the time of study entry. The patients are randomly assigned to one of three dosing regimens in ratio of 2:1:1. Thus, the total number of patients is 80-40 to receive mirtazapine alone, 20 to receive placebo and 20 to receive mirtazapine and zonisamide. In each dosing group, those who terminate early from the study are replaced.

The criteria for inclusion in the study are: (1) Patients must have the ability to give informed consent; (2) Each patient must have an AHI of 1040, severity defined as mild (10-20), moderate (20-30), or severe (3040), using a strict definition of hypopnoea; (3) Patients must be at least 21 years of age; (4) Each patient must have a body mass index (BMI) of no more than 34; (5) Current CPAP users (patients with no more than 3 days of CPAP use in the last 12 months are allowed) are excluded; (6) Patients must all be non-smokers, with no history of smoking for ≧two years; (7) Each patient must have a baseline Epworth Sleep Scale of greater than 10; (8) patients must be able to read and speak English; (9) All females must be either postmenopausal (no menses for at least 1 year) or status-post hysterectomy or oophorectomy (bilateral) or, if of childbearing potential, must have a negative urine pregnancy test prior to randomization, and be using a medically acceptable form of contraception (e.g., hormonal birth control, IUD, double barrier [male condom, female condom, diaphragm] or a barrier method plus a spermicidal agent [contraceptive foam, jelly or cream]).

Primary criteria for exclusion from the study are: (1) Clinically significant comorbidity, including any unstable cardiovascular, gastrointestinal, metabolic, pulmonary (e.g., asthma, COPD), renal, neurological, hepatic, hematologic, immunologic, endocrine, and/or neoplastic disease based on Principal Investigator judgment; (2) Hypertension (those patients with controlled hypertension-systolic <=140 and diastolic <=90—for at least 3 months prior to Tx0/Baseline are allowed to enrol); (3) Patients with evidence of active liver disease (levels of AST, ALT and/or alkaline phosphatase >2× the upper limit of the normal range (ULN) for the laboratory performing the test; (4) Patients with a white blood count below normal range or a count >1.5× the ULN; (5) Patients with anaemia as defined by haemoglobin <80% lower limit of normal; (6) Patients with impaired renal function as evidenced by a creatine value >1.2×ULN; (7) Any patient with a documented history of allergy to sulfonamides (e.g., sulfamethoxazole as part of trimethoprin-sulfamethoxazole combinations); (8) Patients having severe craniofacial abnormalities; (9) Patients having previous or current history of a generalized or partial seizure disorder, or current treatment for any form of seizure disorder (excluding seizure during childhood); (10) Concomitant use of any stimulant medications, including modafinil; (11) Concomitant use sedative hypnotics, tranquilizers, antihistamines (non-sedating antihistamines are allowed), benzodiazepines, or clonidine; (12) Concomitant use of anticonvulsant medication(s); (13) Current diagnosis of any psychiatric illness including any psychotic, schizoaffective, and/or major affective disorder(s) based on DSM-IV criteria; (14) Patients who are receiving concomitant therapy with MAO-A or -B inhibitors, tricyclics, tetracyclics, SSRI agents, NARI agents, SNRI agents, alpha-agonists, or St. John's Wort; (15) Current diagnosis of any substance abuse disorder based on DSM-IV criteria; (16) Current seizure disorder; (17) Pregnant or lactating females; (18) Concomitant use of drugs having known Cytochrome P450 induction or inhibition properties.

Patients are randomly assigned to one of the following groups:

Group 1: mirtazapine 15 mg p.o. nocte+placebo nocte;

Group 2: mirtazapine 15 mg p.o. nocte+100 mg zonisamide p.o. nocte; and

Group 3: placebo nocte+placebo nocte.

Zonisamide is provided as an over-encapsulated 100 mg capsule for oral administration.

Patients are evaluated according to the following efficacy and safety criteria:

Efficacy: (1) Change in calculated AHI score, baseline to follow-up; (2) Change in Epworth Sleep Scale (ESS) score; (3) Patient Global Impression of Change (PGIC); (4) AusEd Driving Simulator performance change; (5) SF-36 score change, total and subdomain; (6) FOSQ score change; (7) Karolinska Sleep Scale score change; (8) Beck Depression Inventory; (9) Beck Anxiety Inventory; (10) Multidimensional Fatigue Inventory; (11) Craving Scale for Australia; (12) Change in Weight.

Safety: (1) Medical history; (2) Vital signs (height, weight, heart rate, respirations, blood pressure (standing and supine)); (3) Urine pregnancy test for females of childbearing potential; (4) Severity and relationship of adverse events to study drugs; (5) Death and other serious adverse events; (6) Discontinuations due to adverse events.

The evaluation is carried out according to the schedule set forth in Table 2, following:

Timing of Treatments and Procedures

TABLE 2
Timing of Treatments and Procedures

| | | | Day 3 | | Day 21 | |
|---|---|---|---|---|---|---|
| | | | (±1 day) | T × 2/Day | (±1 day) | T × 4/Day |
| | | | Telephone | 15 | Telephone | 29/Early |
| | T × 0/ | | Contact | (±3 days) | Contact | Termination |
| Procedure | Baseline | Day 1 | Only | Early AM | Only | (±5 days) |
| Informed consent | X | | | | | |
| Inclusion/exclusion | X | | | | | |
| criteria | | | | | | |
| Medical history | X | | | | | |
| Blood draw for | X | | | X | | X |
| diagnostic | | | | | | |
| laboratories | | | | | | |
| (metabolic/liver | | | | | | |
| profile, CBC, kidney | | | | | | |
| function)^{a} | | | | | | |
| Vital signs, height^{b}, | X | | | X | | X |
| weight | | | | | | |
| Pregnancy test^{c} | X | | | | | X |
| Diagnostic Sleep | X | | | | | X |
| Laboratory | | | | | | |
| Assessment^{d} | | | | | | |
| Epworth Sleep Scale | X | | | X | | X |
| Subjective Snoring | | | | X | | X |
| Questionnaire | | | | | | |
| AusEd Driving | X | | | X | | X |
| Simulator^{e} | | | | | | |
| Dispense home | X | | | X | | |
| breathing monitoring | | | | | | |
| device^{f} | | | | | | |
| SF36 | X | | | | | X |
| Beck Anxiety | X | | | | | X |
| Inventory | | | | | | |
| Multidimensional | X | | | | | X |
| Fatigue Inventory | | | | | | |
| Craving Scale for | X | | | X | | X |
| Australia | | | | | | |
| Karolinska Sleep | X | | | X | | X |
| Scale | | | | | | |
| Beck Depression | X | | | | | X |
| Inventory | | | | | | |
| FOSQ | X | | | | | X |
| PGIC | | | | X | | X |
| Concomitant | X | | X | X | X | X |
| medications | | | | | | |
| Adverse events | X | | X | X | X | X |
| Drug Dispensation^{g} | | X | | X | | |
| Drug Accountability | | | | X | | X |

^{a}Applicable Clinical Laboratory results within 2 weeks prior to the Baseline visit are allowed for use as the Baseline Clinical Laboratory Results.
^{b}Height is measured only at screening. Vital signs, with the exception of height, are performed twice during visits requiring Diagnostic Sleep Laboratory Assessment.
^{c}Urine Pregnancy tests are done only on females of childbearing potential (i.e., females who are not surgically sterile or are <2 years postmenopausal).
^{d}Sleep Lab results from testing within 4 weeks prior to the Baseline visit are allowed for use as the Baseline Sleep Lab requirement.
^{e}The AusEd driving simulator must be performed in the morning, preferably following the PSG (or use of home breathing monitoring device if PSG is not performed at the baseline visit).
^{f}A home breathing monitoring device is dispensed to the patient at T × 0, and T × 2. Used devices are collected by the site at T × 2, and T × 4. The patient must complete an at home evaluation with the home breathing device prior to receipt of drug.
^{g}Drug for a total two weeks on study is dispensed at Day 1 (this visit should directly follow the patient's use of their assigned home monitoring device, either the ARES or Flow Wizard for one night), and at T × 2.

Patients are assigned by a 2:1:1 ratio to one of the following treatment groups: (1) Group 1: mirtazapine 15 mg PO nocte+placebo PO nocte; (2) Group 2: mirtazapine 15 mg PO nocte+100 mg zonisamide PO nocte; (3) Group 3: placebo PO nocte+placebo PO nocte.

Study procedures: Timing of procedures: (1) Patient enrolment procedures, including Baseline/Tx0 and Day 1. (2) Patients sign the informed consent. (3) The physician reviews the medical history of the patient. (4) The physician reviews the admission criteria to determine that the patient is appropriate for the study. (5) Clinical laboratory blood samples are drawn. Patients who have had the applicable blood samples drawn within 2 weeks of signing the informed consent do not need to have blood samples redrawn. The physician must assure that clinical laboratory results are reviewed prior to drug dispensation. (6) A urine pregnancy test is performed on female patients of childbearing potential (i.e., females who are not surgically sterile or are <2 years postmenopausal). (7) A sleep laboratory evaluation is scheduled within 4 weeks unless the patient has had a sleep laboratory diagnosis within the previous 4 weeks. (8) Vital signs are recorded. (9) The patients complete study questionnaires. (10) Patients are trained on the AusEd driving simulator. (11) Patients complete the AusEd driving simulator in the morning. (12) Study personnel review subject AHI. (13) The physician assures that the subject continues to meet admission criteria. (14) The patient is trained in the use of the breathing monitoring devices. (15) The patient uses the home breathing monitoring device for at least one night prior to drug dispensation. (16) The patient is assigned to a dose group. (16) Drug is dispensed.

On-Study Procedures, Tx2/Day 14: (1) Patients return to the clinic in the a.m. (2) Clinical laboratory samples are drawn. (3) Vital signs are collected. (4) Adverse events and concomitant medication use are assessed. (5) Patient questionnaires are completed. (6) The AusEd driving simulator task is completed. (7) New home breathing monitoring devices are dispensed. (8) Drug is dispensed to the patient.

Telephone Contact, Day 3 and Day 21: Patients are contacted at study Days 3and 21 to assess adverse events, review concomitant medications and home device usage.

Exit evaluation, Tx4/Day 28: At the patients final study visit, Tx4/Day 28 or Early Termination the following procedures are performed: (1) Patients return to the clinic in the evening. (2) The home breathing monitoring devices are collected. (3) Clinical laboratory samples are drawn. (4) A urine pregnancy test is performed on female patients of childbearing potential (i.e., females who are not surgically sterile or are <2 years postmenopausal). (5) Vital signs are collected. (6) Adverse events and concomitant medication use are assessed. (7) Patient questionnaires are completed. (9) A sleep laboratory evaluation is performed. (10) The AusEd driving simulator task is completed.

Change in AHI Score: The patient's AHI score is based on results of a diagnostic Sleep Laboratory to be conducted at Tx0/Baseline and Tx4/Day 29.

Change in score of the Epworth Sleep Scale: Each patient completes the modified Epworth Sleep Scale questionnaire at Tx0/Baseline, Tx2/Day 15, and Tx4/Day 29. The questionnaire is designed to evaluate daytime sleepiness.

Change in score of the Karolinska Sleep Scale (KSS). Each patient completes the Karolinska Sleep Scale at Tx0/Baseline, Tx2/Day 15, and Tx4/Day 29.

Patient Global Impression of Change (PGIC): Each patient completes an assessment of patient global impression of change at Tx4/Day 29.

SF-36. Each patient completes the SF-36 questionnaire at Tx0/Baseline and Tx4/Day 29.

FOSQ. Each patient completes the FOSQ at Tx0/Baseline and Tx4/Day 29.

Beck Depression Inventory (BDI). Each patient completes the BDI at Tx0/Baseline and Tx4/Day 29.

Beck Anxiety Inventory (BAI). Each patient completes the BAI at Tx0/Baseline and Tx4/Day 29.

Craving Scale for Australia. Each patient completes the Craving Scale at Tx0/Baseline, Tx2, and Tx4/Day 29.

AusEd Driving Simulator. The AusEd driving simulator evolved as a joint research project between the Sleep Units of St Vincent's Sleep Disorders Service, Royal North Shore Hospital Sydney, Australia and the Respiratory and Sleep Research Unit at the Royal Infirmary Edinburgh, Scotland. The simulator is computer based. The replication of a usual rural road at night allows for the assessment of reaction times through a braking exercise, speed deviation and road deviation. The AusEd is unique as most other simulator programs have used more stimulating protocols potentially alerting drivers while driving alone.

This is a tool tailored to assess three areas of driving performance. These areas are: tracking error measured by steering accuracy, divided attention task by velocity deviation and reaction time. The simulator is installed on a PC with windows NT operating system, with a 212 computer screen, a Thrustmaster T2 steering wheel and pedals (Hillsboro, Oreg., USA) and computer speakers.

Subjects are asked to drive for 30 min, keeping the speed between 60-80 units/hr and to maintain the vehicle in the middle of the left hand lane. Braking reaction time is assessed by 10 trucks appearing intermittently throughout the drive. Like a real vehicle, the simulator is “driven” using a steering wheel, acceleration and brake pedals. The simulated task is designed for monotonous night-time driving on rural road. The visual field is from the driver's seat, looking forward, low beam lighting of a dual carriageway highway. All lighting is turned off during the test sessions. In addition, a low frequency of 60 dB, engine-like noise accompanies the duration of the drive. A standardized driving route is selected based on previous studies in our laboratory, whereby straight section and chicanes or curved parts are 5/7 and 2/7 of the road. This route is used throughout all test sessions. A five-minute practice run is given at the beginning of each test round to minimize practice effect.

The subject controls the simulator by the accelerator, brake pedal and steering wheel. The driving simulator run is on a dual lane highway at night, where forward vision is limited to the equivalent of lights on ‘low beam’. Ten slowly moving trucks going in the same direction as the driver appear during the 30 minute drive. There are no other vehicles going in either direction. Reflective markers are found on both sides of the road, which is standard in Australia for many rural roads. A 60 dB simulated engine noise of a low continuous frequency is played through the computer speakers throughout the drive time. Each subject has an identical presentation of trucks, area of straight road ( 5/7) and chicanes ( 2/7). In the top left hand corner of the monitor is the speedometer, which is red, indicating that the subjects is either driving >80 kms/per hour or <60 kms/per hour. The background on the screen is black (to simulate night time). The markers at the side of the road and line markers in the centre of the road are white and easily visible.

Two experimental home breathing monitoring devices are being evaluated in this study. Patients enrolled in this protocol receive either the ARES or the Flow Wizard at their initial clinic visit. The patients are asked to record between three and five nights per week of sleep breathing data. Each device can be used for three nights of data recording, and the patient is then instructed to bring the devices back to the clinic for their Tx2 or Tx4 visit. At the Tx0 visit, patients are dispensed one device, either one Flow Wizard or one ARES. At the Day 1 and Tx2 visits, they are dispensed two additional devices if they were successful using the first device dispensed at Tx0. Whichever device, the ARES or Flow Wizard, which the patient is assigned at their Tx0 visit, remains the type that is dispensed throughout the course of the patient's study participation.

These devices are used to supplement the data obtained from the sleep laboratory diagnostic evaluations.

The ARES and Flow Wizard devices, and the uses thereof, are described in Example 2, above.

Blood samples for hematology and clinical chemistry assessment are obtained at Baseline, Tx2, and Tx4 (study end) or early termination. Patients must satisfy all entry criteria prior to entering into the baseline phase of this trial.

Any laboratory parameter that the investigator believes to be clinically significant is recorded as an adverse event. As expected for all adverse events, clinically significant laboratory abnormalities are followed until clinical resolution, improvement or stabilization.

Hematology assessments include red blood cell count (RBC), hemoglobin (Hgb), hematocrit (Hct), mean corpuscular volume (MCV), mean corpuscular hemoglobin (MCH), mean corpuscular hemoglobin concentration (MCHC), white blood cell count (WBC) including differential (neutrophils, lymphocytes, monocytes, eosinophils, and basophils), and platelet count. Blood samples obtained at the study site are analyzed by the local site laboratory.

Chemistry assessments include sodium, potassium, chloride, carbon dioxide (CO2), calcium, phosphorus, glucose, blood urea nitrogen (BUN), creatinine, alkaline phosphatase (AP), total bilirubin, direct bilirubin, aspartate aminotransferase (AST), alanine aminotransferase (ALT), lactate dehydrogenase (LDH), total protein, albumin, triglyceride, cholesterol, and uric acid. Blood samples obtained at the study site are analyzed by the local site laboratory.

A urine β-human chorionic gonadotropin hormone sample for pregnancy testing is performed by the site, using an in-office test kit at randomization and Tx4/early termination for all females of child bearing potential, as defined by the inclusion criteria.

Standing and supine blood pressure and heart rate, weight and temperature are obtained at each clinic visits; height are also obtained at the baseline visit.

An Adverse Event or Adverse Experience (AE) is any untoward medical occurrence in a patient or clinical investigation subject administered a pharmaceutical product. It is not necessary that the AE have a causal relationship to treatment with the product.

An AE therefore is any unfavorable and unintended sign (for example, a clinically significant abnormal laboratory finding) symptom, disease, or worsening in current medical conditions whether or not considered related to study medication.

While patients are encouraged to complete the study, they may voluntarily withdraw at any time. The Principal Investigator provides written documentation on the appropriate Case Report Form describing the reason for discontinuation. If a patient withdraws or is discontinued from the treatment before completion, every effort should be made to complete the assessments scheduled for the Tx5 visit. For safety reasons, efforts must be made to follow patients for at least 30 days following their discontinuation. The sponsor should be notified of all study withdrawals in a timely manner.

This protocol is designed to evaluate mirtazapine, a drug currently approved for treatment of depression in Australia and many other parts of the world, alone and in conjunction with zonisamide. A placebo tablet (for the mirtazapine only and placebo only groups) is provided by the University of Sydney. Mirtazapine is purchased commercially. Mirtazapine is available as a 15 mg white tablet. Mirtazapine is taken orally once daily approximately 0.5 to 1 hour before bedtime (nocte). A matched placebo is provided by the University of Sydney. Zonisamide is imported from overseas. Zonisamide is available as a 100 mg white and red hard gelatin capsule. Zonisamide is taken orally once daily approximately 0.5 to 1 hour before bedtime.

The required amount of each study drug per patient is packaged in blister packs and secondary packaging is in patient kit form. Drug is dispensed from the patient kit by the pharmacist at each study visit.

Concomitant use of any stimulant medication, sedative hypnotics, tranquilizers, antihistamines (non-sedating antihistamines are allowed), benzodiazepines, or clonidine are not permitted during this study. In addition, concomitant therapy with MAO-A or -B inhibitors, tricyclics, tetracyclics, SSRI agents, NARI agents, SNRI agents or alpha-agonists are not permitted during this study.

The results of the foregoing study demonstrate that combination therapy with mirtazapine and zonisamide is effective in the treatment of sleep related breathing disorders, such as sleep apnea and snoring.

The foregoing examples are presented for illustrative purposes only. The person skilled in the art will recognize that the invention is not limited by the foregoing examples and that other embodiments are possible within the scope of the present invention.

What is claimed is:

1. A method of treating a sleep related breathing disorder, comprising administering to a patient a therapeutically effective combination of mirtazapine and a second pharmaceutically active ingredient which increases upper airway muscle tone during sleep and stabilizes respiratory drive.

2. The method of claim 1, wherein the second pharmaceutically active ingredient is zonisamide.

3. The method of claim 1, wherein the sleep related breathing disorder is sleep apnea.

4. A composition comprising mirtazapine and a second pharmaceutically active ingredient which increases upper airway muscle tone during sleep and stabilizes respiratory drive.

5. The composition of claim 4, wherein the second pharmaceutically active ingredient is zonisamide.

6. A kit comprising a first dosage form comprising mirtazapine and a second dosage form comprising a second active pharmaceutical ingredient which increases upper airway muscle tone during sleep and stabilizes respiratory drive.

7. The kit of claim 5, wherein the second active pharmaceutical ingredient is zonisamide.

8. A method of treating a sleep related breathing disorder, comprising administering to a patient a therapeutically effective combination of mirtazapine and a second pharmaceutically active ingredient which stabilizes respiratory drive.

9. The method of claim 8, wherein the second pharmaceutically active ingredient is selected from the group consisting of topiramate, zonisamide and a 5HT1A agonist.

10. The method of claim 9, wherein the 5HT1A agonist is buspirone.

11. The method of claim 8, wherein the second pharmaceutically active ingredient is administered in a separate dosage form from the mirtazapine.

12. The method of claim 11, wherein the second pharmaceutically active ingredient is administered at a different time from the mirtazapine.

13. A composition comprising mirtazapine and a second pharmaceutically active ingredient which stabilizes respiratory drive.

14. The composition of claim 13, wherein the second pharmaceutically active ingredient is selected from the group consisting of topiramate, zonisamide and a 5HT1A agonist.

15. The composition of claim 14, wherein the 5HT1A agonist is buspirone.

16. A kit comprising a first dosage form containing mirtazapine and a second dosage form comprising a second pharmaceutically active ingredient which stabilizes respiratory drive.

17. The kit according to claim 16, wherein the second pharmaceutically active ingredient is selected from the group consisting of topiramate, zonisamide and a 5HT1A agonist.

18. The kit of claim 17, wherein the 5HT1A agonist is buspirone.

19. A method of treating a sleep related breathing disorder, comprising administering to a patient a therapeutically effective combination of mirtazapine and a second pharmaceutically active ingredient which treats one of the sequelae of sleep apnea.

20. The method of claim 19, wherein the sequelae of sleep apnea include daytime drowsiness.

21. The method of claim 20, wherein the second pharmaceutically active ingredient comprises a compound selected from the group consisting of amantadine, bupropion, modafinil, r-modafinil, and SDZ-NVI-085 and amphetamine.

22. A kit comprising a first dosage form comprising mirtazapine and a second dosage form comprising a second pharmaceutically active ingredient which treats one of the sequelae of sleep apnea.

23. The kit of claim 22, wherein the second pharmaceutically active ingredient is selected from the group consisting of amantadine, bupropion, modafinil, r-modafinil, and SDZ-NVI-085 and amphetamine.

24. A method of treating a sleep-related breathing disorder, comprising administering to a patient a therapeutically effective amount of a composition comprising mirtazapine and zonisamide.

25. The method of claim 24, wherein the sleep-related breathing disorder is sleep apnea.

26. A method of treating a sleep-related breathing disorder, comprising administering to a patient a therapeutically effective amount of a composition comprising mirtazapine and zonisamide.

27. The method of claim 26, wherein the sleep-related breathing disorder is sleep apnea.

28. A method of treating a sleep-related breathing disorder, comprising administering to a patient a first composition comprising mirtazapine and a second composition comprising zonisamide.

29. The method of claim 28, wherein the sleep-related breathing disorder is sleep apnea.

30. A method of improving patient tolerance to positive airway pressure therapy, comprising administering to the patient a therapeutically effective amount of a composition comprising mirtazapine.

31. The method of claim 30, wherein the composition further comprises zonisamide.

32. A method of improving patient tolerance to positive airway pressure therapy, comprising administering to the patient a first composition comprising mirtazapine and a second composition comprising zonisamide.

33. A therapeutic composition for the treatment of a sleep-related breathing disorder, comprising a therapeutically effective amount of mirtazapine.

34. The composition of claim 33, wherein the sleep-related breathing disorder is sleep apnea.

35. A method of treating a sleep-related breathing disorder, comprising administering to a patient a therapeutically effective amount of a composition comprising milnacipran.

36. The method of claim 35, wherein the composition further comprises zonisamide.

37. The method of claim 36, wherein the sleep-related breathing disorder is sleep apnea.

38. A method of treating a sleep-related breathing disorder, comprising administering to a patient a first composition comprising milnacipran and a second composition comprising zonisamide.

39. The method of claim 38, wherein the sleep-related breathing disorder is sleep apnea.

40. A method of improving patient response to positive airway pressure therapy, comprising administering to a patient therapeutically effective amount of a composition comprising milnacipran.

41. The method of claim 40, wherein the composition further comprises zonisamide.

42. A kit for the treatment of a sleep-related breathing disorder, comprising a first dosage form comprising milnacipran and a second dosage form comprising zonisamide.

43. The kit of claim 42, wherein the sleep-related breathing disorder is sleep-apnea.

44. A pharmaceutical composition for the treatment of a sleep-related breathing disorder, comprising a therapeutically effective amount of a composition comprising milnacipran.

45. The composition of claim 44, wherein the sleep-related breathing disorder is sleep apnea.

46. A pharmaceutical composition for the treatment of a sleep-related breathing disorder, comprising a therapeutically effective amount of a combination of zonisamide and milnacipran.

47. The composition of claim 46, wherein the sleep-related breathing disorder is sleep apnea.

48. A method of treating a sleep related breathing disorder, comprising administering to a patient a therapeutically effective amount of mirtazapine and amantadine.

49. The method of claim 48, wherein mirtazapine and amantadine are administered in separate dosage forms.

50. The method of claim 49, wherein the sleep related breathing disorder is sleep apnea.

51. A method of treating a sleep related breathing disorder, comprising administering to a patient a therapeutically effective amount of mirtazapine and buproprion.

52. The method of claim 51, wherein mirtazapine and buproprion are administered in separate dosage forms.

53. The method of claim 32, wherein the sleep related breathing disorder is sleep apnea.

54. A method of treating a sleep related breathing disorder, comprising administering to a patient a therapeutically effective amount of mirtazapine and modafinil.

55. The method of claim 54, wherein mirtazapine and modafinil are administered in separate dosage forms.

56. The method of claim 54, wherein the sleep related breathing disorder is sleep apnea.
